(19) United States
(12) Patent Application Publication
(10) Pub. No.: US 2024/0360100 A1
(43) Pub. Date: Oct. 31, 2024

(54) HETEROCYCLIC COMPOUNDS AS SEROTONIN (5-HT) 5-HT2A AND 5-HT2C RECEPTOR POSITIVE ALLOSTERIC MODULATORS

(71) Applicant: The Board of Regents of The University of Texas System, Austin, TX (US)

(72) Inventors: Jia Zhou, Galveston, TX (US); Kathryn A. Cunningham, Galveston, TX (US); Noelle C. Anastasio, Galveston, TX (US); Andrew A. Bolinger, Galveston, TX (US)

(21) Appl. No.: 18/684,645

(22) PCT Filed: Aug. 18, 2022
(86) PCT No.: PCT/US22/40817

§ 371 Date: Feb. 16, 2024

Related U.S. Application Data
(60) Provisional application No. 63/304,323, filed on Jan. 28, 2022.
(60) Provisional application No. 63/234,581, filed on Aug. 18, 2021.

(51) Int. Cl. C07D 401/12; A61K 31/4525; A61K 31/454; A61K 31/4545; A61K 31/496; A61K 31/5377; A61K 31/541; C07D 211/60; C07D 405/12; C07D 413/12; C07D 417/12

(52) U.S. Cl. CPC C07D 401/12; A61K 31/4525; A61K 31/454; A61K 31/4545; A61K 31/496; A61K 31/5377; A61K 31/541; C07D 211/60; C07D 405/12; C07D 413/12; C07D 417/12

(57) ABSTRACT

Disclosed herein are serotonin (5-hydroxytryptamine) 5-HT_{2A} receptor and/or 5-HT_{2C} receptor allosteric modulators, the preparation thereof and use thereof.

### CROSS REFERENCE TO RELATED APPLICATIONS

This application claims the benefit of U.S. Provisional Appl. No. 63/234,581, filed Aug. 18, 2021, and U.S. Provisional Appl. No. 63/304,323, filed Jan. 28, 2022. The contents of the aforesaid applications are relied upon and are incorporated by reference herein in their entirety.

### STATEMENT OF FEDERALLY SPONSORED RESEARCH AND DEVELOPMENT

This invention was made with government support under Grant Nos. R21 MH093844, R01 DA038446, and T32 DA07287, awarded by the NIH. The government has certain rights in the invention.

### FIELD OF THE INVENTION

The field of the invention relates generally to novel small molecules that bind and/or modulate different forms of serotonin (5-HT) 5-HT2A and 5-HT2C receptors as well as the preparation and the use thereof.

### BACKGROUND

Serotonin (5-HT) is an endogenous small molecule which enacts remarkably pleiotropic actions in the central and peripheral nervous systems as well as immune, intestinal, and cardiovascular systems. Serotonin binds to 14 receptors arrayed within the plasma membrane of cells resident in these systems; these receptors are grouped into six families of G protein coupled receptors (GPCRs) and one ligand-gated ion channel. The 5-HT₂R family is composed of triplets (5-HT_{2A}R, 5-HT_{2B}R, 5-HT_{2C}R) and subtype selectivity is a major determining factor regarding the scale of its potential therapeutic utility (Barnes et al., Pharmacological Reviews, 73:310-520, 2021). The discovery of increasingly selective receptor ligands has shown that each 5-HT₂R mediates a myriad of functions even though these receptors are related in their molecular structure, amino acid sequences and signaling properties. Of note, 5-HT_{2C}R signaling is a key component of the mechanisms of action underlying the cognitive and behavioral dimensions of substance use disorders (SUDs), particularly well-described for cocaine use disorder (CUD) (Cunningham et al., Handbook of the Behavioral Neurobiology of Serotonin, 2020; Howell and Cunningham, Pharmacological Reviews, 67:176-197, 2015). On the other hand, the 5-HT_{2A}R mediates antipsychotic drug effects, inflammation and pain, and even viral entry into cells and post-viral brain sequela (Elphick et al., Science, 306:1380-1383, 2004; Abbott et al., Neuroscience, 77:575-584, 1997; Flanagan et al., Life Sciences, 236:116790; Couch et al., Neuroimage, 75:177-186, 2013; Couch et al., PloS One, 10:e0130643). The 5-HT_{2A}R is very well characterized as a key mediator of the perceptual and psychedelic states, and mood and cognitive alterations evoked by psychedelics such as d-lysergic acid diethylamide (LSD) and psilocybin (Nichols, Pharmacology and Therapeutics, 101:131-181, 2004; Orejarena et al., International Journal of Neuropsychopharmacology, 14:927-940, 2011). In short, the quest for novel chemical entities through targeting 5-HT_{2C}R and 5-HT_{2A}R actions promises new therapeutic gains in managing a myriad of disorders.

Ligands that selectively enrich 5-HT₂R function may be useful in combating impaired serotonergic control that contributes to mental health disorders, SUDs, pain, and inflammatory disorders. The classic approach is to be the development of agonists targeted to bind directly to the orthosteric binding pocket. However, as is the case for the 5-HT₂R family, the orthosteric site has co-evolved with 5-HT and is highly conserved among 5-HT₂R members. Thus, selectively targeting the orthosteric site of the 5-HT_{2A}R among its closely related subtypes, 5-HT_{2B}R and 5-HT_{2C}R, has proven to be challenging (Barnes et al., Pharmacological Reviews, 73:310-520, 2021). Targeted 5-HT_{2A}R, as well as 5-HT_{2C}R, allosteric modulation provides a pointed approach that broadens the available small molecule toolbox for maximizing neuropsychiatric health.

### DESCRIPTION

### 1.0 Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

The term “alkenyl” as used herein by itself or as part of another group refers to both straight and branched chain radicals. In one embodiment, the alkenyl group has 3-12 carbons. In another embodiment, the alkenyl group has 3-7 carbons. In another embodiment, the alkenyl group has 3-6 carbons. In another embodiment, the alkenyl group has 3-4 carbons (also referred to as “C₃₋₄ alkenyl” or “C3-4 alkenyl”). Alkenyl groups can include, for example, allyl, 2-methallyl, butenyl, -cis-2-butenyl, trans-2-butenyl, 3-pentenyl, and isoprenyl.

The term “alkyl” as used herein by itself or as part of another group refers to both straight and branched chain radicals. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons (also referred to as “Cₗ₋₄ alkyl” or “C1-4 alkyl”). The term “alkyl” may include methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, and dodecyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkylene” as used herein refers to straight and branched chain alkyl linking groups, i.e., an alkyl group that links one group to another group in a molecule. In some embodiments, the term “alkylene” may include —(CH₂)n- where n is 2-8.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

In certain embodiments, the term “cycloalkyl” includes bicyclic ring systems. The bicyclic ring system may be in the form of a bridged, fused, or spiro form.

The term “aryl” refers to a phenyl group, which may be unsubstituted or substituted by one or more groups selected from alkyl, halogen, haloalkyl, hydroxy, alkoxy, carbonyl, alkylamido, nitro, amino, dialkylamino, carboxy, thio or thioalkyl.

An “amino” group refers to an —NH₂ group.

An “amido” group refers to an —CONH₂ group. An alkylamido group refers to an —CONHR group wherein R is as defined above. A dialkylamido group refers to an —CONRR′ group wherein R and R′ are as defined above.

The term “halogen” or “halo” as used herein by itself or as part of another group refers to chlorine, bromine, fluorine or iodine.

The term “hydroxy” or “hydroxyl” as used herein by itself or as part of another group refers to an —OH group.

An “alkoxy” group refers to an —O-alkyl group wherein “alkyl” is as defined above.

A “thio” group refers to an —SH group.

An “alkylthio” group refers to an —SR group wherein R is alkyl as defined above.

The term “heteroaryl” as used herein refers to groups having 5 to 14 ring atoms; 6, 10 or 14 7π-electrons shared in a cyclic array; and containing carbon atoms and 1, 2 or 3 oxygen, nitrogen or sulfur heteroatoms. The heteroaryl moiety may be unsubstituted or substituted by one or more groups selected from halogen, haloalkyl, hydroxy, alkoxy, carbonyl, alkylamido, nitro, amino, dialkylamino, carboxy, thio or thioalkyl. Examples of heteroaryl groups include thienyl, imadizolyl, oxadiazolyl, isoxazolyl, triazolyl, pyridyl, pyrimidinyl, pyridazinyl, furyl, pyranyl, thianthrenyl, pyrazolyl, pyrazinyl, indolizinyl, isoindolyl, isobenzofuranyl, benzoxazolyl, xanthenyl, 2H-pyrrolyl, pyrrolyl, 3H-indolyl, indolyl, indazolyl, purinyl, 4H-quinolizinyl, isoquinolyl, quinolyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furazanyl, and phenoxazinyl groups. Especially preferred heteroaryl groups include 1,2,3-triazole, 1,2,4-triazole, 5-amino 1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

The term “heterocycle” or “heterocyclic ring”, as used herein except where noted, represents a stable 3- to 8-membered monocyclic-, or stable 7- to 11-membered bicyclic heterocyclic ring system, any ring of which may be saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. Rings may contain one oxygen or sulfur, one to three nitrogen atoms, or one oxygen or sulfur combined with one or two nitrogen atoms. The heterocyclic ring may be attached at any heteroatom or carbon atom that results in the creation of a stable structure. Further, “heterocycle” or “heterocyclic ring” moiety may be unsubstituted or substituted by one or more groups selected from halogen, haloalkyl, hydroxy, alkoxy, carbonyl, alkylamido, nitro, amino, dialkylamino, carboxy, thio or thioalkyl. Examples of such heterocyclic groups include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

In certain embodiments, the term “heterocycle” includes bicyclic ring systems. The bicyclic ring system may be in the form of a bridged, fused, or spiro form.

The term “alkylamino” as used herein by itself or as part of another group refers to an amino group which is substituted with one alkyl group having from 1 to 6 carbon atoms. The term “dialkylamino” as used herein by itself or as part of another group refers to an amino group which is substituted with two alkyl groups, each having from 1 to 6 carbon atoms.

As used herein, the term “arylalkyl” denotes an alkylene, group substituted with an aryl group, for example, Ph-(CH₂)ₙ—, where n is 1-6, Ph-(CH₂)₃—, Ph-(CH₂)₂— etc.

A “therapeutically effective amount” is an amount sufficient to decrease, prevent or ameliorate the symptoms associated with a medical condition.

The term “non-hydrogen substituent” refers to a substituent that is not made up solely of hydrogen. Examples of non-hydrogen substituents includes halogen, C₁₋₄ alkyl, C₃₋₈ alkenyl, halogen-substituted C₁₋₄ alkyl, NR⁹R¹⁰, and CN. In some embodiments, non-hydrogen substituent includes methyl. In further embodiments, non-hydrogen substituent includes fluoride (F). In further embodiments, non-hydrogen substituent includes NH₂.

Various groups are described above, and below, as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, alkyl, heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl (—C(O)NR₂), unsubstituted alkyl, unsubstituted heteroalkyl, alkoxy, alkylthio, alkylamino, (alkyl)₂amino, alkylsulfinyl, alkyl sulfonyl, aryl sulfonyl, sulfinyl, sulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO2, —S(C₁₋₄alkyl), —SO₂(C₁₋₄alkyl), —CO₂(C₁₋₄alkyl), —SO₂—, and —O(C₁₋₄alkyl).

The term “administering” or “administration” refers to contacting one or more cells of a subject (including human, horse, cat, dog, monkey, rat, and mice) with one or more compounds according to the present invention. In some embodiments, administration may occur in vitro. In further embodiments, administration may occur in vivo.

### BRIEF DESCRIPTION OF DRAWINGS

FIG. 1 shows the structure of compound AB0113, an exemplary embodiment of the invention, and graphs A and B show the compound's modulation activity in 5-HT_{2A}R—CHO cells and 5-HT_{2C}R—CHO cells, respectively.

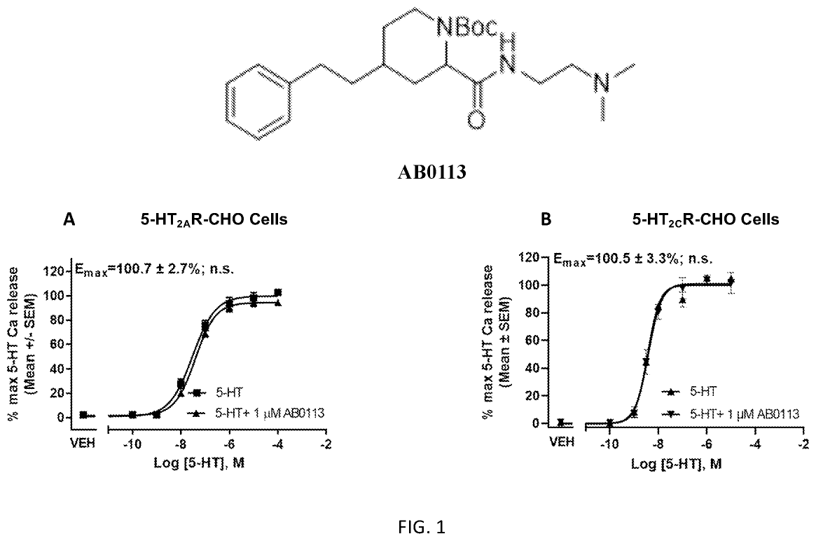

FIG. 2 shows the structure of compound AB0116, an exemplary embodiment of the invention, and graphs A and B show the compound's modulation activity in 5-HT_{2A}R—CHO cells and 5-HT_{2C}R—CHO cells, respectively.

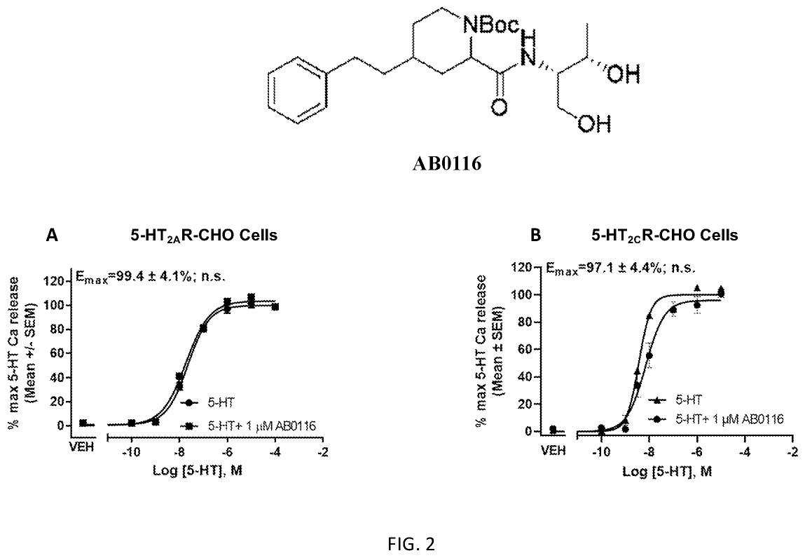

FIG. 3 shows the structure of compound AB0121, an exemplary embodiment of the invention, and graphs A and B show the compound's modulation activity in 5-HT_{2A}R—CHO cells and 5-HT_{2C}R—CHO cells, respectively.

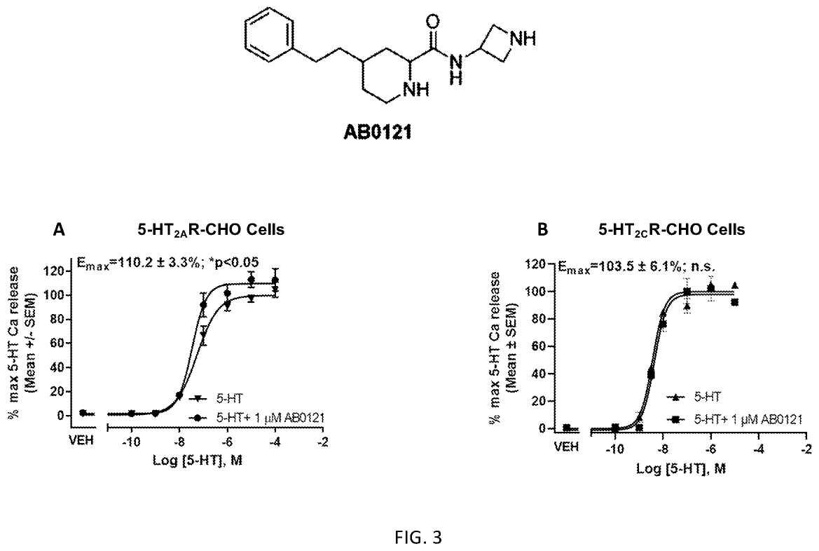

FIG. 4 shows the structure of compound AB0122, an exemplary embodiment of the invention, and graphs A and B show the compound's modulation activity in 5-HT_{2A}R—CHO cells and 5-HT_{2C}R—CHO cells, respectively.

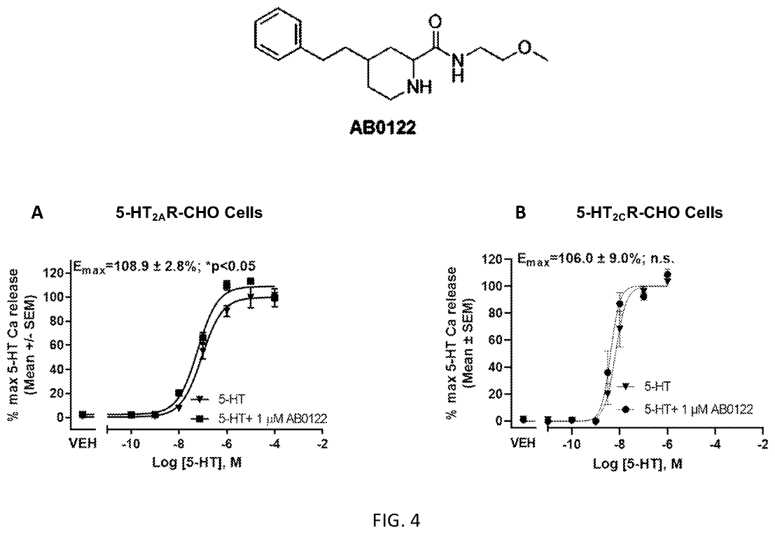

FIG. 5 shows the structure of compound AB0123, an exemplary embodiment of the invention, and graphs A and B show the compound's modulation activity in 5-HT_{2A}R—CHO cells and 5-HT_{2C}R—CHO cells, respectively.

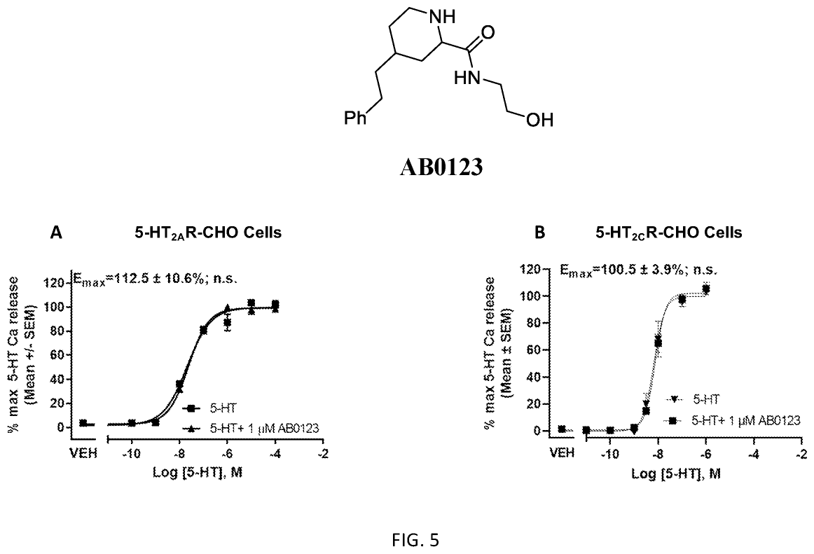

FIG. 6 shows the structure of compound AB0124, an exemplary embodiment of the invention, and graphs A and B show the compound's modulation activity in 5-HT_{2A}R—CHO cells and 5-HT_{2C}R—CHO cells, respectively.

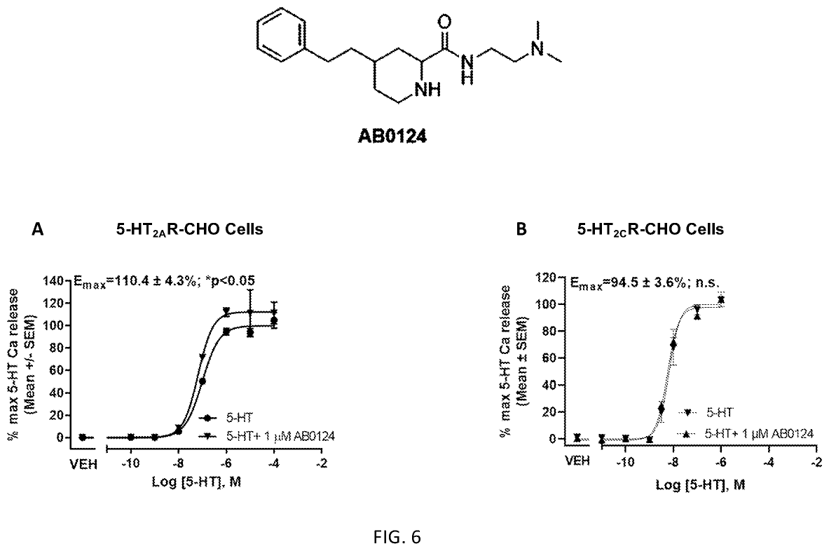

FIG. 7 shows the structure of compound AB0125, an exemplary embodiment of the invention, and graphs A and B show the compound's modulation activity in 5-HT_{2A}R—CHO cells and 5-HT_{2C}R—CHO cells, respectively.

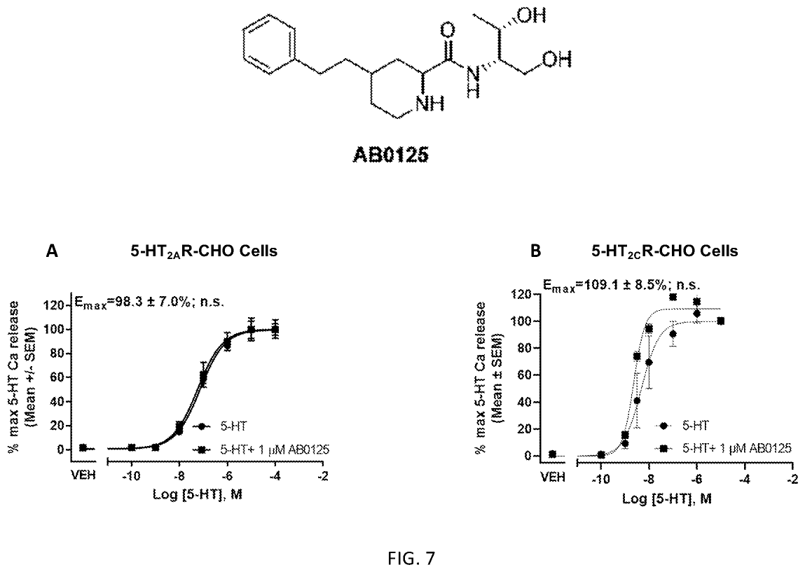

FIG. 8 shows the structure of compound AB0126, an exemplary embodiment of the invention, and graphs A and B show the compound's modulation activity in 5-HT_{2A}R—CHO cells and 5-HT_{2C}R—CHO cells, respectively.

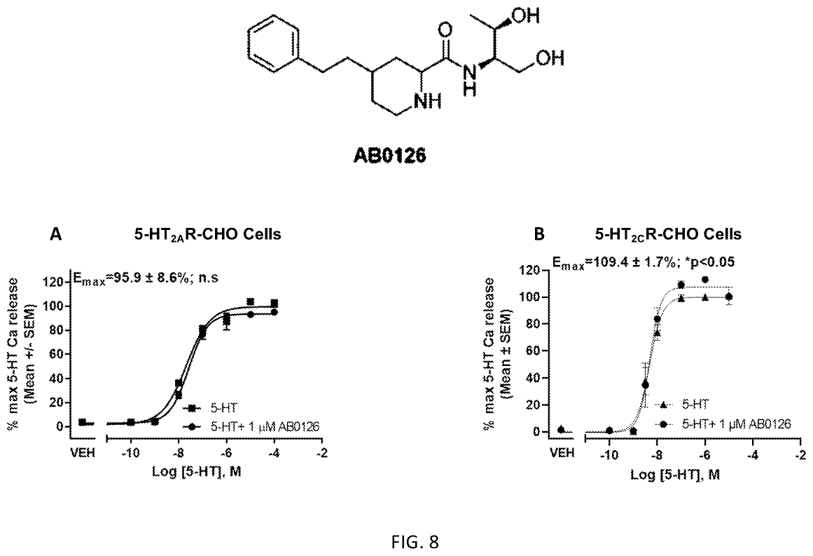

FIG. 9 shows the structure of compound AB0127, an exemplary embodiment of the invention, and graphs A and B show the compound's modulation activity in 5-HT_{2A}R—CHO cells and 5-HT_{2C}R—CHO cells, respectively.

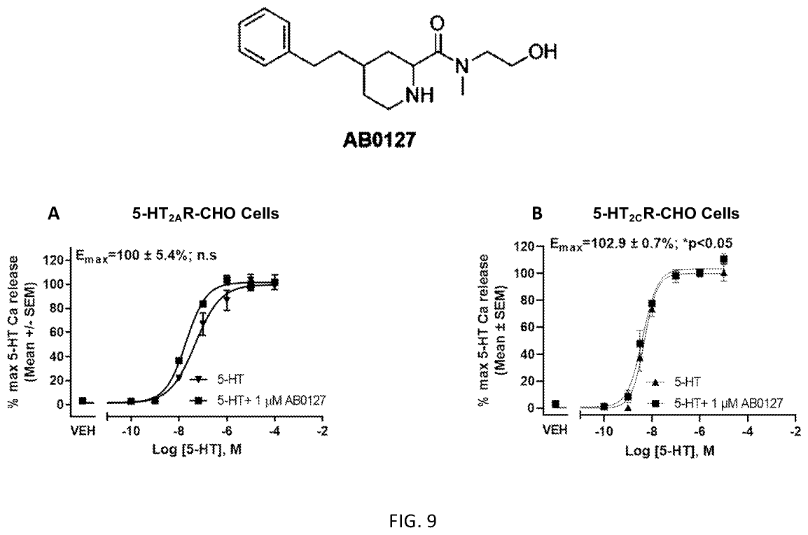

FIG. 10 shows the structure of compound AB0128, an exemplary embodiment of the invention, and graphs A and B show the compound's modulation activity in 5-HT_{2A}R—CHO cells and 5-HT_{2C}R—CHO cells, respectively.

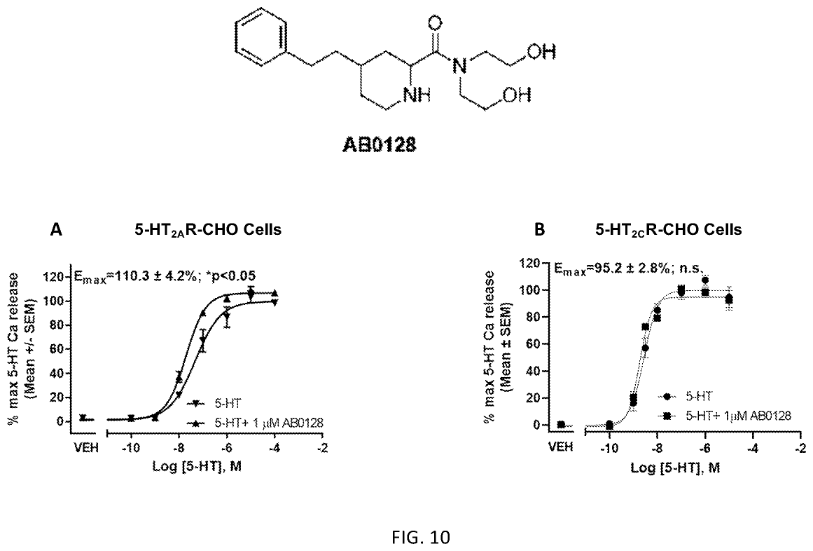

FIG. 11 shows the structure of compound AB0131, an exemplary embodiment of the invention, and graphs A and B show the compound's modulation activity in 5-HT_{2A}R—CHO cells and 5-HT_{2C}R—CHO cells, respectively.

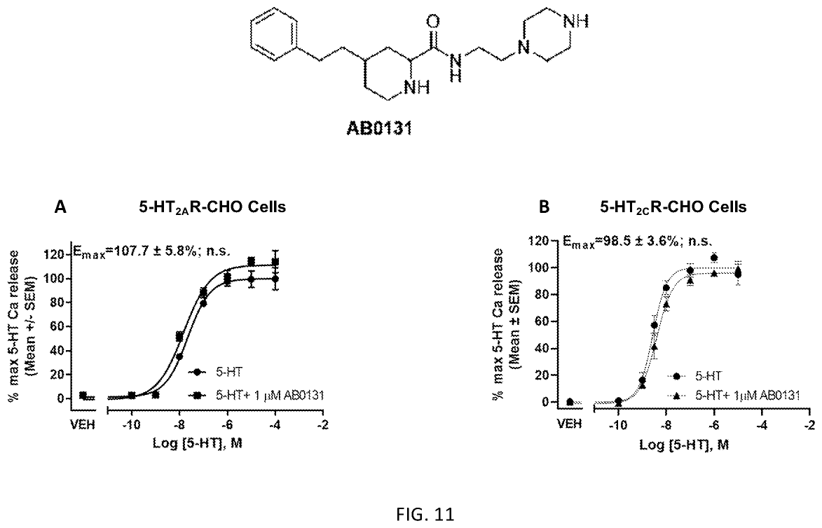

FIG. 12 shows the structure of compound AB0132, an exemplary embodiment of the invention, and graphs A and B show the compound's modulation activity in 5-HT_{2A}R—CHO cells and 5-HT_{2C}R—CHO cells, respectively.

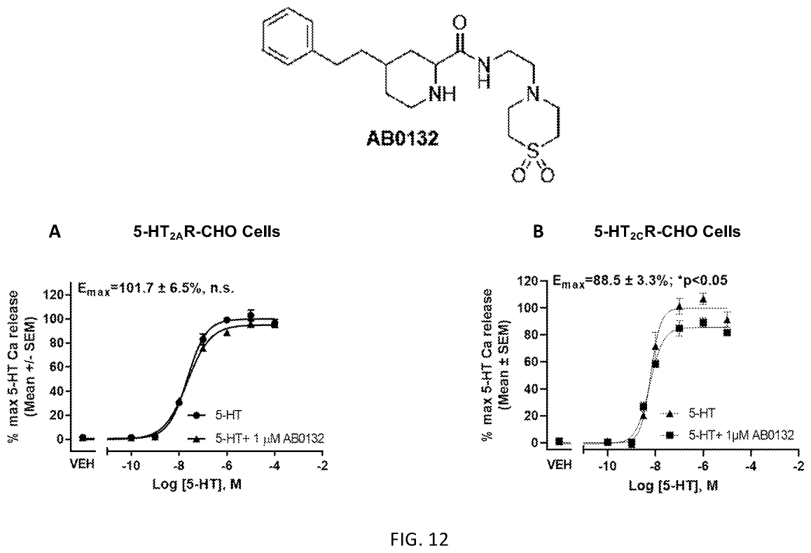

FIG. 13 shows the structure of compound AB0135, an exemplary embodiment of the invention, and graphs A and B show the compound's modulation activity in 5-HT_{2A}R—CHO cells and 5-HT_{2C}R—CHO cells, respectively.

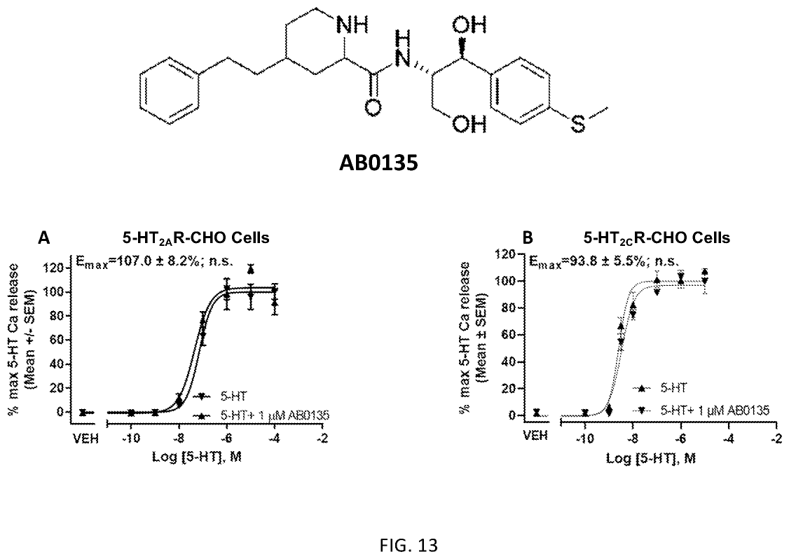

FIG. 14 shows the structure of compound AB0136, an exemplary embodiment of the invention, and graphs A and B show the compound's modulation activity in 5-HT_{2A}R—CHO cells and 5-HT_{2C}R—CHO cells, respectively.

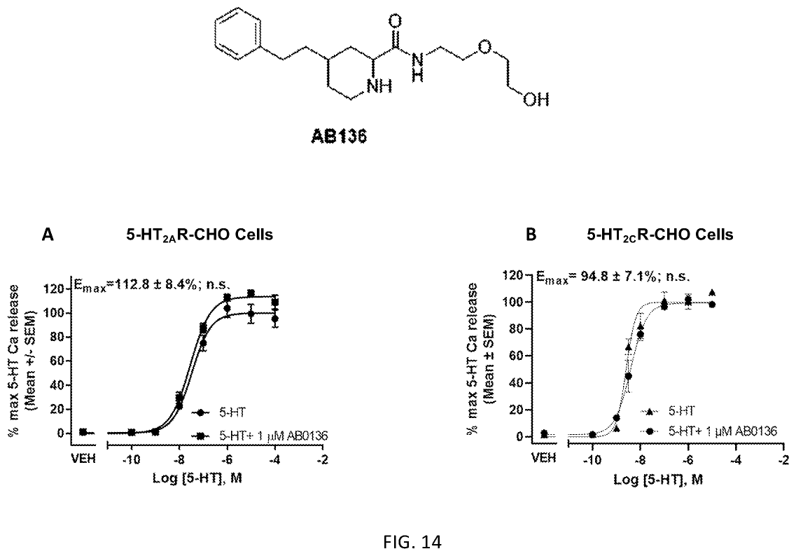

FIG. 15 shows the structure of compound AB0145, an exemplary embodiment of the invention, and graphs A and B show the compound's modulation activity in 5-HT_{2A}R—CHO cells and 5-HT_{2C}R—CHO cells, respectively.

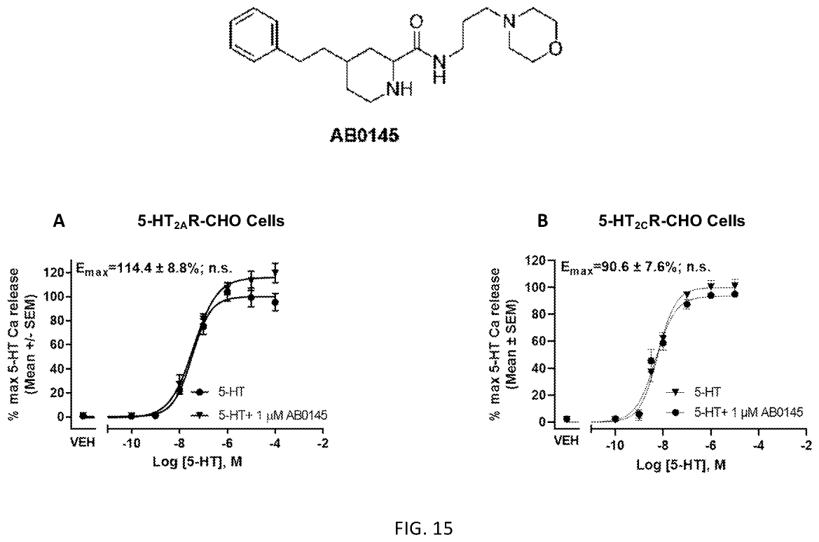

FIG. 16 shows the structure of compound AB0146, an exemplary embodiment of the invention, and graphs A and B show the compound's modulation activity in 5-HT_{2A}R—CHO cells and 5-HT_{2C}R—CHO cells, respectively.

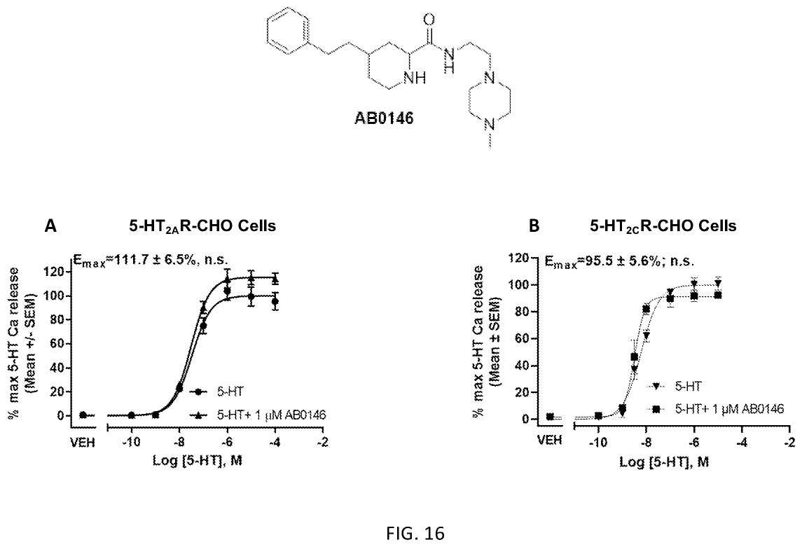

FIG. 17 shows the structures of CTW0415, CTW0456, and CTW0508 (exemplary embodiments of the invention) and a table of Ca²⁺ induction data.

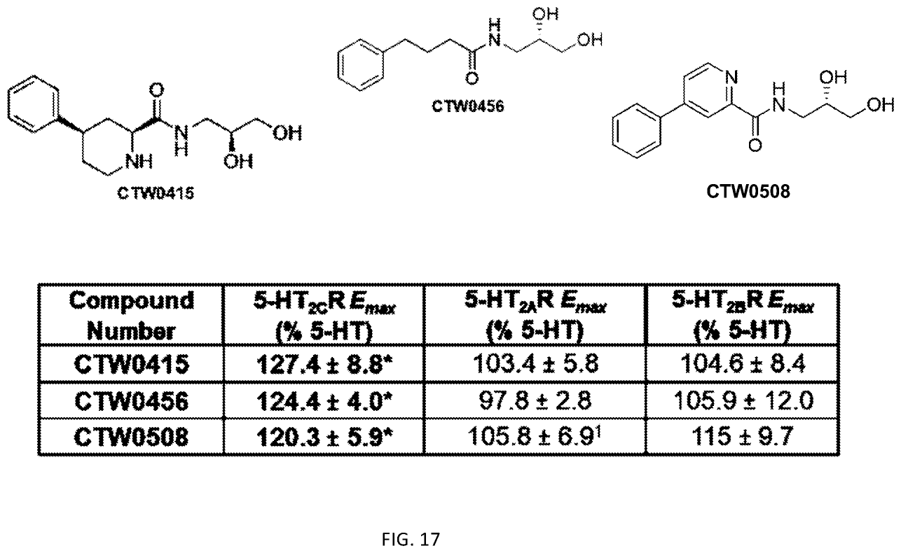

FIG. 18 show the structure of compound CTW0508, an exemplary embodiment of the invention a graph “A” showing data for 5-HT-induced IP1 accumulation, and a graph “B” showing pERK/1/2 in h5-HT_{2C}R—CHO cells.

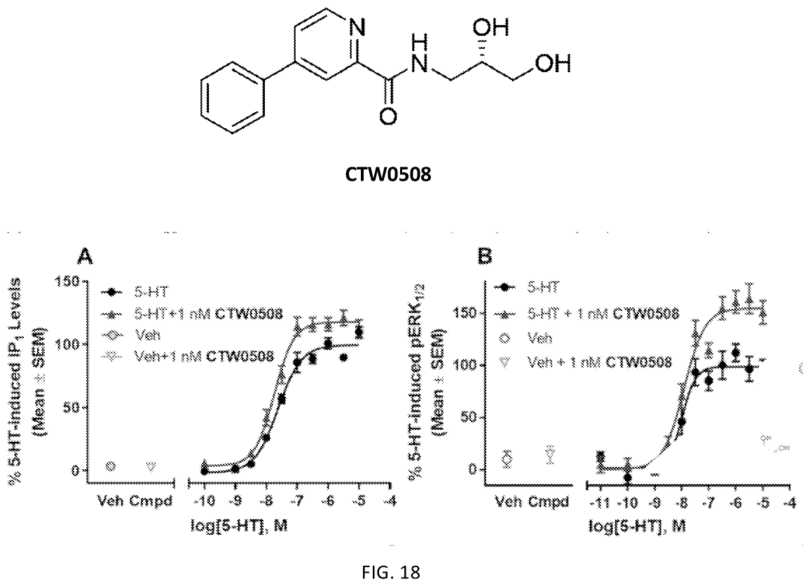

FIG. 19 shows Chiral HPLC Chromatogram for CTW0404

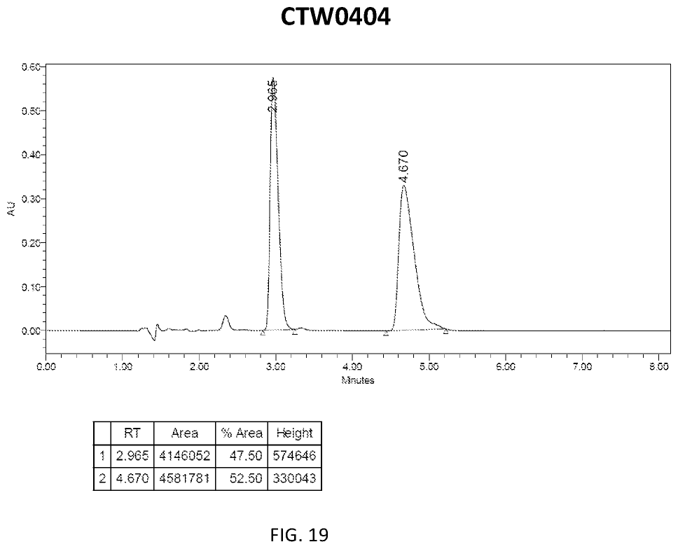

FIG. 20 shows Chiral HPLC Chromatogram for CTW0404-P1

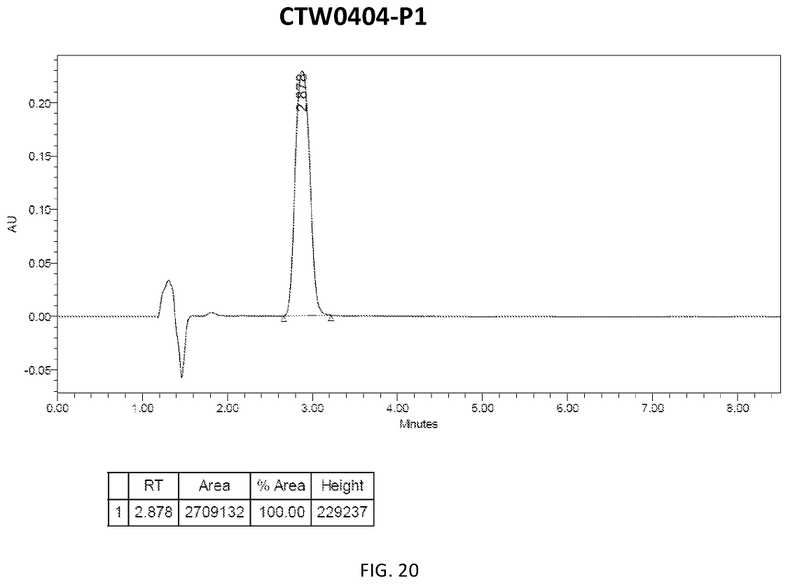

FIG. 21 shows Chiral HPLC Chromatogram for CTW0404-P2

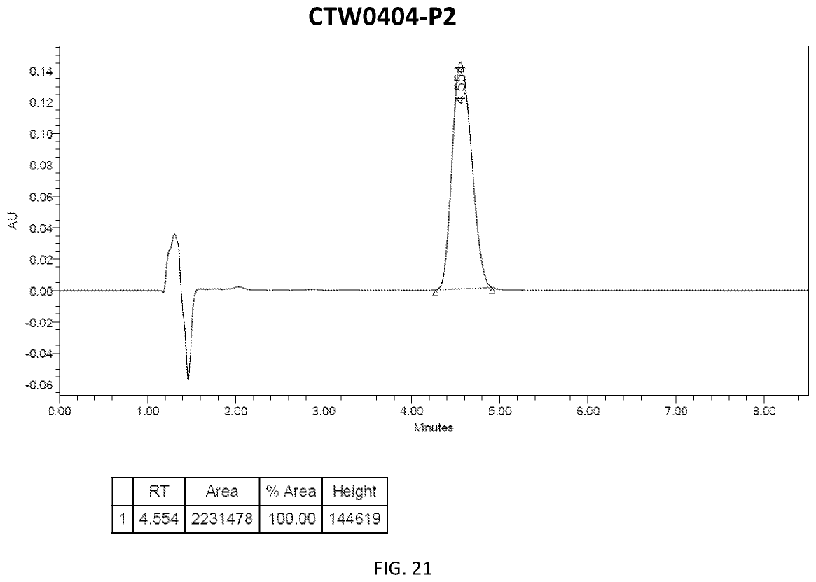

FIG. 22 shows Chiral HPLC Chromatogram for CTW0419

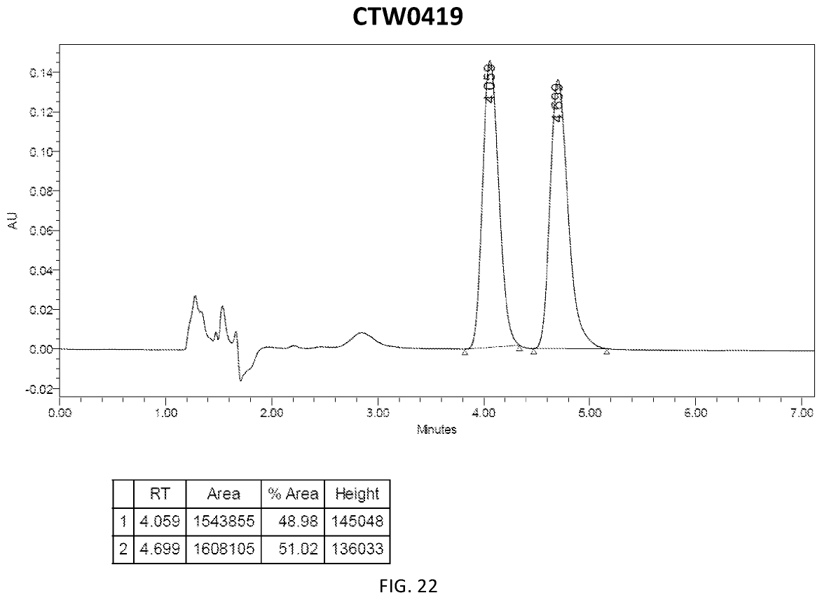

FIG. 23 shows Chiral HPLC Chromatogram for CTW0419-P1

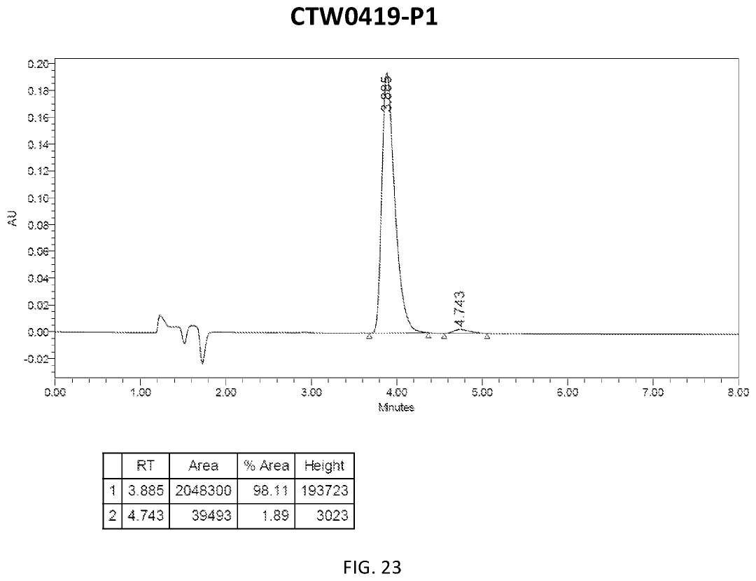

FIG. 24 shows Chiral HPLC Chromatogram for CTW0419-P2

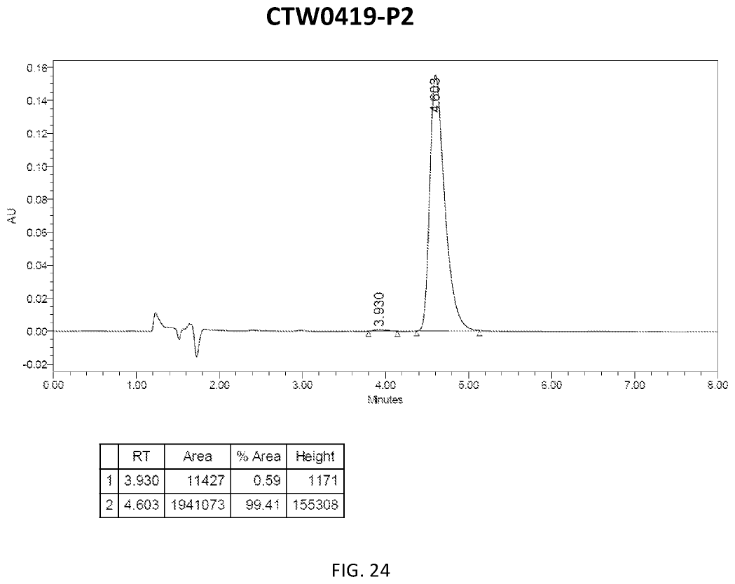

In recent years, allosteric modulators of several GPCRs have been developed and are predicted to have robust promise in the treatment of a variety of biological disorders (May et al., Annu. Rev. Pharmacol. Toxicol., 47:1-51, 2007). The recent preclinical indications of efficacy, coupled with the launch of cinacalcet and maraviroc as the first marketed GPCR allosteric modulators, validate the clinical utility of both positive and negative allosteric modulators (Conn et al., Nature Reviews Drug Discovery, 8:41-54, 2009). The studies reported to date provide proof of concept that fuels the discovery of highly selective allosteric ligands for other GPCRs.

In biochemistry, allostery is the regulation of an enzyme or other protein by binding an effector molecule at an allosteric site(s) on the protein (that is, a site other than the active site of that protein). Thus, a regulatory site of an allosteric protein is physically distinct from the binding site. Effectors that enhance protein function are referred to as positive allosteric modulators (PAMs), whereas those that decrease protein function are called negative allosteric modulators (NAMs) (Christopoulos, Molecular Pharmacology, 86:463-478, 2014). Neutral allosteric ligand (NAL) refers to an allosteric modulator that binds to the allosteric site but has no effects on the response to the orthosteric ligand. (Christopoulos, Molecular Pharmacology, 86:463-478, 2014). For example, the compounds described herein are 5-HT_{2A}R and/or 5-HT_{2C}R PAMs that enhance 5-HT_{2A}R and/or 5-HT_{2C}R function. The compounds can be probes for understanding the biology of these receptors to ultimately develop therapeutics for the treatment of diseases, including, but not limited to mental health disorders (e.g., anxiety, depression, schizophrenia), SUDs, eating disorders and obesity, pain, and inflammatory disorders. Targeting allosteric modulation of the 5-hydroxytryptamine receptor (5-HTR) to identify novel CNS probes with the potential for therapeutic application offers pharmacological advantages to a direct agonist or antagonist approach.

The 5-HTRs are a group of GPCRs and ligand-gated ion channels found in the brain and periphery that mediate important key functions in biology. The 5-HTR family includes 5-HT₁ to 5-HT₇ with each type having numerous receptor subtypes. The 5-HTRs modulate the release of many neurotransmitters, including glutamate, GABA, dopamine, epinephrine/norepinephrine, and acetylcholine, as well as many hormones, including oxytocin, prolactin, vasopressin, cortisol, corticotropin, and substance P. The 5-HTRs influence various biological and neurological processes such as aggression, anxiety, appetite, cognition, immunological function, learning, memory, mood, nausea, pain, sleep, and thermoregulation; and are the target of a variety of pharmaceutical and illicit drugs, including many antidepressants, antipsychotics, anorectics, antiemetics, gastroprokinetic agents, antimigraine agents, hallucinogens, and entactogens.

Without wishing to be bound by a particular theory, one premise is that selective enrichment of 5-HT_{2C}R function will combat vulnerability to cocaine use disorder (CUD) and relapse during recovery. Targeting of the 5-HT_{2C}R remains challenging since each of the 14 5-HT GPCR subtypes share a conserved orthosteric binding site for the endogenous agonist 5-HT.

By targeting a binding site that is spatially distinct from the conserved 5-HT orthosteric site, divergent residues or topological surfaces may be exploited to achieve 5-HT_{2C}R selectivity while enhancing the functional response to 5-HT. Allosteric modulators are being discovered to bind to an identified, spatially distinct allosteric site to selectively potentiate 5-HT_{2C}R, but not 5-HT_{2A}R or 5-HT_{2B}R, signaling in vitro without intrinsic activity at any of these receptors.

One aspect of the invention pertains to compounds identified as allosteric modulators of 5-HT_{2A}R and/or 5-HT_{2C}R, as well as pharmaceutical compositions and methods using the same. Certain embodiments also include methods of identifying and methods of synthesizing the compounds as well as the isomer separations of compounds with chiral centers. Optimization and development of allosteric 5-HT_{2A}R and/or 5-HT_{2C}R modulators that bind sites other than the primary orthosteric ligand binding site generate novel, highly selective, and potent ligands of 5-HT_{2A}R and/or 5-HT_{2C}R. Such molecules may be used as small molecule probes for the study of receptor biology and as effective therapeutics for a variety of diseases.

Another aspect of the present invention provides several highly potent ligands as selective allosteric modulators of 5-HT_{2A}R and/or 5-HT_{2C}R with positive, negative, or neutral allosteric modulator activity. For example, two 5-HT_{2C}R PAMs (CYD-1-79, CTW0415) potentiate in vivo effects of a full 5-HT_{2C}R agonist in male rats, an effect which was blocked by a selective 5-HT_{2C}R antagonist, verifying reliance on 5-HT_{2C}R function (Wild et al., Journal of Medicinal Chemistry, 62:288-305, 2019; Wold et al., Journal of Medicinal Chemistry, 63:7529-7544, 2020). Surprisingly, first-in-class 5-HT_{2A} RPAMs (CTW0404, CTW0419) have been discovered, providing tools to optimize 5-HT_{2C}R PAMs and 5-HT_{2A}R PAMs with favorable drug-like properties and analyze select molecules in proof-of-concept in vivo assays.

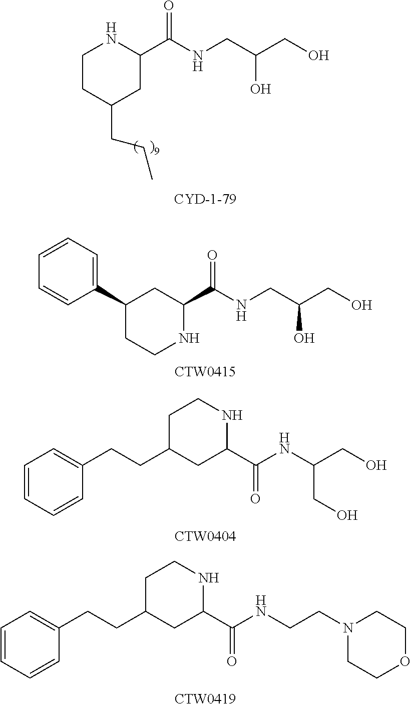

The inventors surprisingly identified unique 5-HT_{2C}R PAM molecules that, for the first time, render this receptor a highly actionable target toward mitigating CUD-associated vulnerabilities. The synthetic route to access the first reported selective 5-HT_{2C}R PAM PNU-69176E and its diastereomer. PNU-69176E potentiated 5-HT-evoked Caᵢ²⁺ release, exhibiting the profile of a 5-HT_{2C}R PAM with agonist-like properties observed at the highest concentration tested in vitro (10 μM). PNU-69176E is a complex molecule with multiple chiral centers, and—a diversification strategy was designed to build on the breakdown of PNU-69176E into three distinct moieties: the central ring, lipophilic tail (LT), and polar head (PH). A first generation of 5-HT_{2C}R PAMs was discovered which bind to an identified, spatially distinct allosteric site to selectively potentiate 5-HT_{2C}R, but not 5-HT_{2A}R or 5-HT_{2B}R, signaling in vitro without intrinsic activity at any of these receptors. As a proof-of-concept, two 5-HT_{2C}R PAMs (CYD-1-79, CTW0415) selectively potentiated in vivo effects of a full 5-HT_{2C}R agonist (Wild et al., Journal of Medicinal Chemistry, 62:288-305, 2019; Wold et al., Journal of Medicinal Chemistry, 63:7529-7544, 2020). CYD-1-79 also suppressed cocaine-seeking in a cocaine use disorder relapse-like behavioral model in male rats (Wild et al., Journal of Medicinal Chemistry, 62:288-305, 2019).

Intriguingly, first-in-class 5-HT_{2A} RPAMs (CTW0414, CTW0419) was also discovered, unlocking an entirely new and innovative line of investigation. CTW0404 and CTW0419 significantly shifted the concentration-response curve for 5-HT-evoked accumulation of the downstream inositol-1,4,5-trisphosphate metabolite inositol monophosphate (IP₁) upward with similar efficacy as Caᵢ²⁺ release in vitro.

### EMBODIMENTS OF COMPOUNDS

The following is a non-limiting list of embodiments of the invention and is meant to be exemplary:
Embodiment 101. A compound according to Formula I

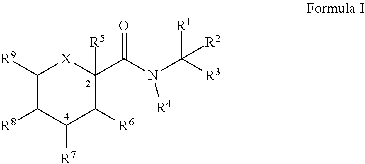

or a pharmaceutically acceptable salt thereof, wherein:
X is —NR— or —O—; R is independently selected from the group consisting of hydrogen, carbamate, or others defined in R¹
R¹, R², R³ and R⁴ are independently selected from the group consisting of hydrogen, substituted or unsubstituted (C₁-C₁₀) alkyl, substituted or unsubstituted (C₁-C₁₀) alkylene, substituted or unsubstituted (C₁-C₁₀) alkenyl, substituted or unsubstituted (C₁-C₁₀) alkynyl, substituted or unsubstituted (C₁-C₁₀) heteroalkyl, hydroxy (C₁-C₁₀) alkyl, amino (C₁-C₁₀) alkyl, (C₁-C₁₀) alkoxy, (C₁-C₁₀) alkoxy (C₁-C₁₀) alkyl, substituted or unsubstituted cycloalkyl, substituted or unsubstituted heterocyclyl, substituted or unsubstituted aryl, and substituted or unsubstituted heteroaryl; or at least two of any of R¹, R² and R³ taken together forms a cycloalkyl or heterocyclyl ring;
R¹, R², R³ and R⁴ are independently substituted with one or more substituents selected from the group consisting of hydrogen, halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl;
R⁵, R⁶, R⁷, R⁸ and R⁹ are independently selected from the group consisting of hydrogen, (C₁-C₁₅) alkyl, (C₁-C₁₅) alkoxy, substituted or unsubstituted (C₁-C₁₅) alkyl aryl, substituted or unsubstituted (C₁-C₁₅) alkyl heteroaryl, substituted or unsubstituted cycloalkyl, substituted or unsubstituted heterocyclyl, substituted or unsubstituted aryl, and substituted or unsubstituted heteroaryl;
with the proviso that when X is —NR—, each of R², R³, R⁴, R⁵, R⁶, R⁸ and R⁹ is hydrogen, and R⁷ is —CH₂CH₂-phenyl, then R¹ is not morpholino-(CH₂)ₘ—, where m is 2 or 3; and
with the proviso that when X is —NR—, each of R³, R⁴, R⁵, R⁶, R⁸ and R⁹ is hydrogen and R⁷ is —CH₂CH₂-phenyl, then R¹ and R² are not both —CH₂OH.
Embodiment 102. The compound according to embodiment 101, wherein R⁴ is selected from hydrogen and (C₁-C₁₀) alkyl.
Embodiment 103. The compound according to embodiment 101, wherein R⁴ is hydrogen.
Embodiment 104. The compound according to embodiment 101, wherein each of R⁵, R⁶, Rand R⁹ is hydrogen.
Embodiment 105. The compound according to embodiment 101, wherein R⁴ taken together with any one of R¹, R², and R³ together forms a nitrogen-containing heterocycle.
Embodiment 106. The compound of embodiment 105, wherein the nitrogen-containing heterocycle is selected from pyrrolidine, piperidine, and piperazine.
Embodiment 107. The compound according to embodiment 101, wherein at least two of any of R¹, R² and R³ taken together form a cycloalkyl or heterocyclyl ring.
Embodiment 108. The compound according to embodiment 101, wherein at least two of any of R¹, R² and R³ taken together form a 5-membered cycloalkyl ring, a 6-membered cycloalkyl ring, a 5-membered heterocyclyl ring, or a 6-membered heterocyclyl ring.
Embodiment 109. The compound according to embodiment 101, wherein at least one of R¹, R², and R³ is hydrogen.
Embodiment 110. The compound according to embodiment 101, wherein at least two of R¹, R², and R³ are hydrogen.
Embodiment 111. The compound according to embodiment 101, wherein each of R⁴, R⁵, R⁶, R⁸ and R⁹ is H and R⁷ is —CH₂CH₂-phenyl.
Embodiment 112. The compound of embodiment 101, wherein R⁵, R⁶, R⁸ and R⁹ are hydrogen and R⁷ is independently selected from the group consisting of hydrogen, (C₁-C₁₅) alkyl, (C₁-C₁₅) alkoxy, substituted or unsubstituted (C₁-C₁₅) alkyl aryl, substituted or unsubstituted (C₁-C₁₅) alkyl heteroaryl, substituted or unsubstituted cycloalkyl, substituted or unsubstituted heterocyclyl, substituted or unsubstituted aryl, and substituted or unsubstituted heteroaryl, and wherein the R⁷ group at position 4 of the piperidine ring and the amide group at position 2 of the same piperidine ring are in a cis configuration.
Embodiment 201. A compound according to Formula II

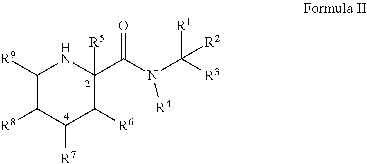

or a pharmaceutically acceptable salt thereof, wherein:
R¹, R², R³ and R⁴ are independently selected from the group consisting of hydrogen, substituted or unsubstituted (C₁-C₁₀) alkyl, substituted or unsubstituted (C₁-C₁₀) alkylene, substituted or unsubstituted (C₁-C₁₀) alkenyl, substituted or unsubstituted (C₁-C₁₀) alkynyl, substituted or unsubstituted (C₁-C₁₀) heteroalkyl, hydroxy (C₁-C₁₀) alkyl, amino (C₁-C₁₀) alkyl, (C₁-C₁₀) alkoxy, (C₁-C₁₀) alkoxy (C₁-C₁₀) alkyl, substituted or unsubstituted cycloalkyl, substituted or unsubstituted heterocyclyl, substituted or unsubstituted aryl, and substituted or unsubstituted heteroaryl;
R¹, R², R³ and R⁴ are independently substituted with one or more substituents selected from the group consisting of hydrogen, halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl; or at least two of any of R¹, R² and R³ taken together form a cycloalkyl or heterocyclyl ring;
R⁵, R⁶, R⁷, R⁸ and R⁹ are independently selected from the group consisting of hydrogen, (C₁-C₁₅) alkyl, (C₁-C₁₅) alkoxy, substituted or unsubstituted (C₁-C₁₅) alkyl aryl, substituted or unsubstituted (C₁-C₁₅) alkyl heteroaryl, substituted or unsubstituted cycloalkyl, substituted or unsubstituted heterocyclyl, substituted or unsubstituted aryl, and substituted or unsubstituted heteroaryl;
with the proviso that when each of R², R³, R⁴, R⁵, R⁶, R⁸ and R⁹ is hydrogen and R⁷ is —CH₂CH₂-phenyl, then R¹ is not morpholino-(CH₂)ₘ—, where m is 2 or 3; and
with the proviso that when each of R³, R⁴, R⁵, R⁶, R⁸ and R⁹ is hydrogen and R⁷ is —CH₂CH₂-phenyl, then R¹ and R² are not both —CH₂OH.
Embodiment 202. The compound according to embodiment 201, wherein R⁴ is selected from hydrogen and (C₁-C₁₀) alkyl.
Embodiment 203. The compound according to embodiment 201, wherein R⁴ is hydrogen.
Embodiment 204. The compound according to embodiment 201, wherein each of R⁵, R⁶, Rand R⁹ is hydrogen.
Embodiment 205. The compound according to embodiment 201, wherein R⁴ and any one of R¹, R², and R³ taken together form a nitrogen-containing heterocycle.
Embodiment 206. The compound of embodiment 205, wherein the nitrogen-containing heterocycle is selected from pyrrolidine, piperidine, and piperazine.
Embodiment 207. The compound according to embodiment 201, wherein at least two of R¹, R² and R³ taken together form a cycloalkyl or heterocyclyl ring.
Embodiment 208. The compound according to embodiment 201, wherein at least two of R¹, R² and R³ taken together form a 5-membered cycloalkyl ring, a 6-membered cycloalkyl ring, a 5-membered heterocyclyl ring, or a 6-membered heterocyclyl ring.
Embodiment 209. The compound according to embodiment 201, wherein at least one of R¹, R², and R³ is hydrogen.
Embodiment 210. The compound according to embodiment 201, wherein at least two of R¹, R², and R³ are hydrogen.
Embodiment 211. The compound according to embodiment 201, wherein each of R², R³, R⁴, R⁵, R, Rand R⁹ is H and R⁷ is —CH₂CH₂-phenyl.
Embodiment 212. The compound according to embodiment 201, wherein the compound has a structure chosen from:

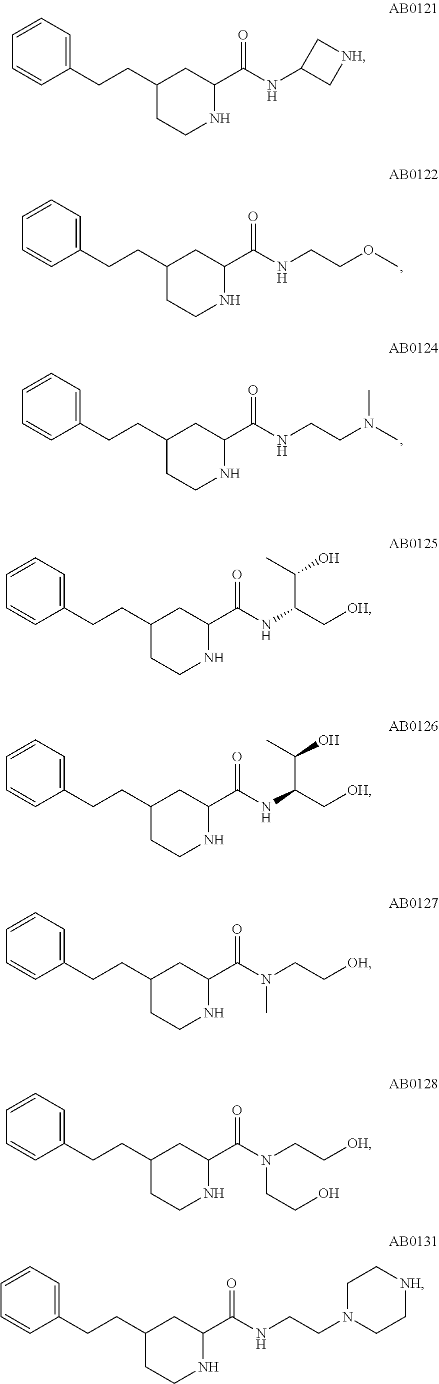

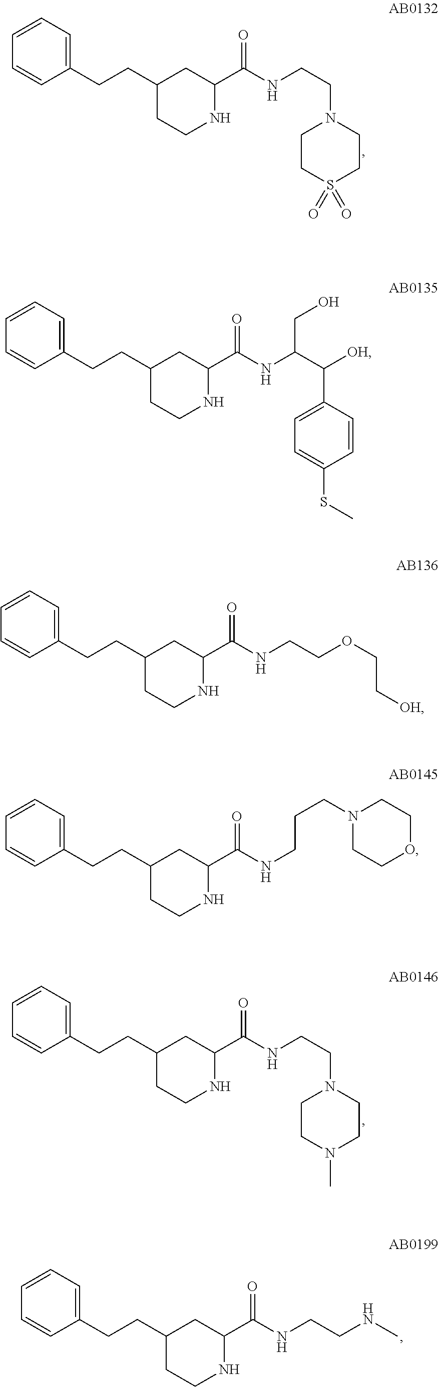 [SMILES: O=C(NCCN1CCS(=O)(=O)CC1)C1CC(CCc2ccccc2)CCN1 ; CSc1ccc(C(O)C(CO)NC(=O)C2CC(CCc3ccccc3)CCN2)cc1 ; O=C(NCCOCCO)C1CC(CCc2ccccc2)CCN1 ; O=C(NCCCN1CCOCC1)C1CC(CCc2ccccc2)CCN1 ; CN1CCN(CCNC(=O)C2CC(CCc3ccccc3)CCN2)CC1 ; CNCCNC(=O)C1CC(CCc2ccccc2)CCN1]

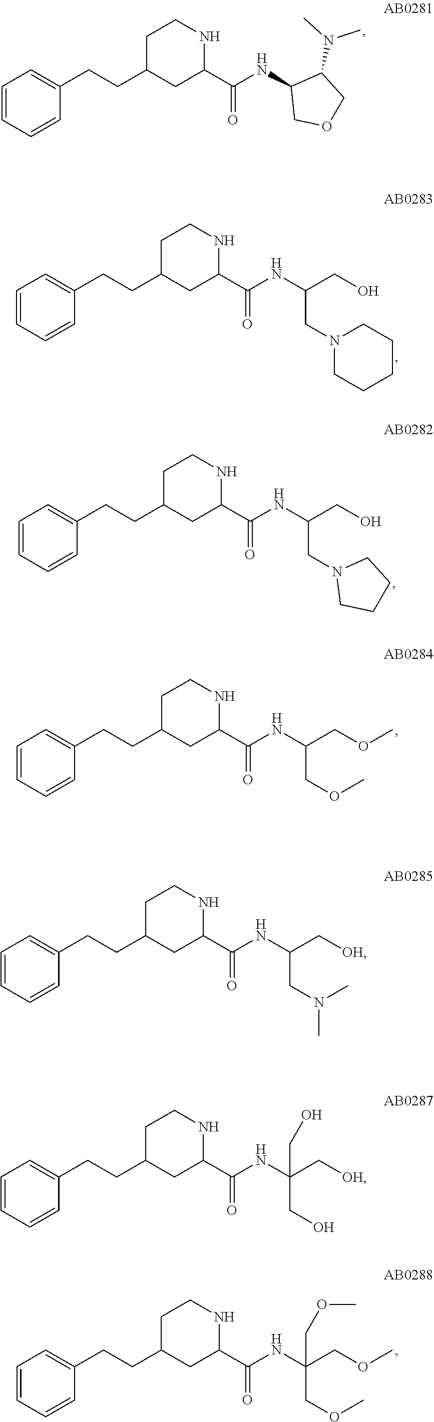

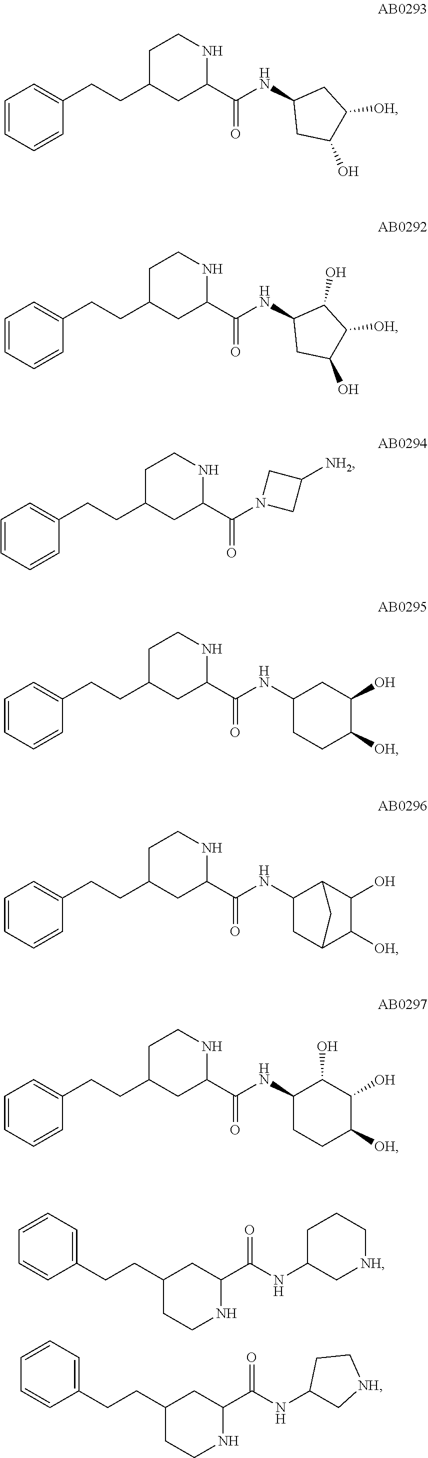

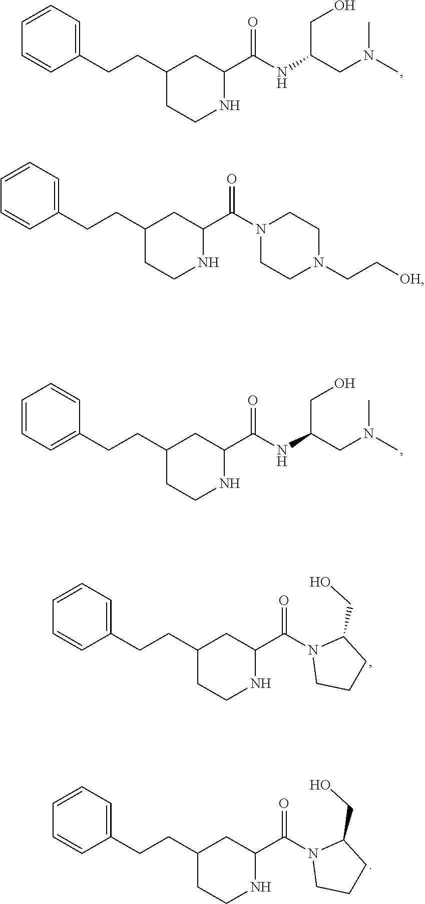 [SMILES: CN(C)C[C@@H](CO)NC(=O)C1CC(CCc2ccccc2)CCN1 ; O=C(C1CC(CCc2ccccc2)CCN1)N1CCN(CCO)CC1 ; CN(C)C[C@H](CO)NC(=O)C1CC(CCc2ccccc2)CCN1 ; O=C(C1CC(CCc2ccccc2)CCN1)N1CCC[C@H]1CO ; O=C(C1CC(CCc2ccccc2)CCN1)N1CCC[C@@H]1CO]

Embodiment 301. A compound according to Formula III

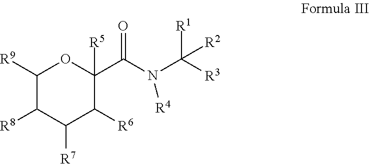

or a pharmaceutically acceptable salt thereof, wherein:
R¹, R², R³ and R⁴ are independently selected from the group consisting of hydrogen, substituted or unsubstituted (C₁-C₁₀) alkyl, substituted or unsubstituted (C₁-C₁₀) alkylene, substituted or unsubstituted (C₁-C₁₀) alkenyl, substituted or unsubstituted (C₁-C₁₀) alkynyl, substituted or unsubstituted (C₁-C₁₀) heteroalkyl, hydroxy (C₁-C₁₀) alkyl, amino (C₁-C₁₀) alkyl, (C₁-C₁₀) alkoxy, (C₁-C₁₀) alkoxy (C₁-C₁₀) alkyl, substituted or unsubstituted cycloalkyl, substituted or unsubstituted heterocyclyl, substituted or unsubstituted aryl, and substituted or unsubstituted heteroaryl; or at least two of any of R¹, R² and R³ taken together form a cycloalkyl or heterocyclyl ring;
R¹, R², R³ and R⁴ are independently substituted with one or more groups selected from the group consisting of hydrogen, halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl;
R⁵, R⁶, R⁷, R⁸ and R⁹ are independently selected from the group consisting of hydrogen, (C₁-C₁₅) alkyl, (C₁-C₁₅) alkoxy, substituted or unsubstituted (C₁-C₁₅) alkyl aryl, substituted or unsubstituted (C₁-C₁₅) alkyl heteroaryl, substituted or unsubstituted cycloalkyl, substituted or unsubstituted heterocyclyl, substituted or unsubstituted aryl, and substituted or unsubstituted heteroaryl.
Embodiment 302. The compound according to embodiment 301, wherein R⁴ is selected from hydrogen and (C₁-C₁₀) alkyl.
Embodiment 303. The compound according to embodiment 301, wherein R⁴ is hydrogen.
Embodiment 304. The compound according to embodiment 301, wherein each of R⁵, R⁶, Rand R⁹ is hydrogen.
Embodiment 305. The compound according to embodiment 301, wherein R⁴ and any one of R¹, R², and R³ taken together form a nitrogen-containing heterocycle.
Embodiment 306. The compound according to embodiment 305, wherein the nitrogen-containing heterocycle is selected from the group consisting of pyrrolidine, piperidine, and piperazine.
Embodiment 307. The compound according to embodiment 301, wherein at least two of R¹, R² and R³ taken together form a cycloalkyl or heterocyclyl ring.
Embodiment 308. The compound according to embodiment 301, wherein at least two of R¹, R² and R³ taken together form a 5-membered cycloalkyl ring, a 6-membered cycloalkyl ring, a 5-membered heterocyclyl ring, or a 6-membered heterocyclyl ring.
Embodiment 309. The compound according to embodiment 301, wherein at least one of R¹, R², and R³ is hydrogen.
Embodiment 310. The compound according to embodiment 301, wherein at least two of R¹, R², and R³ are hydrogen.
Embodiment 311. The compound according to embodiment 301, wherein the compound has a structure chosen from:

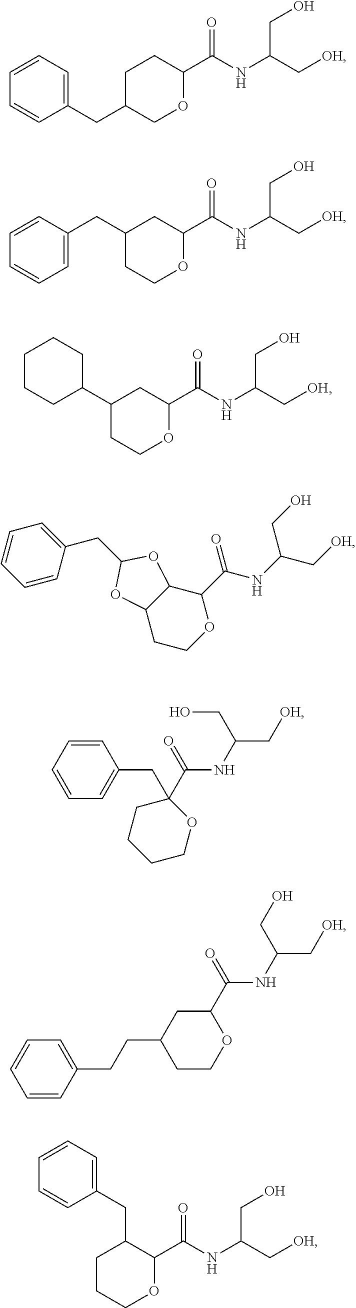

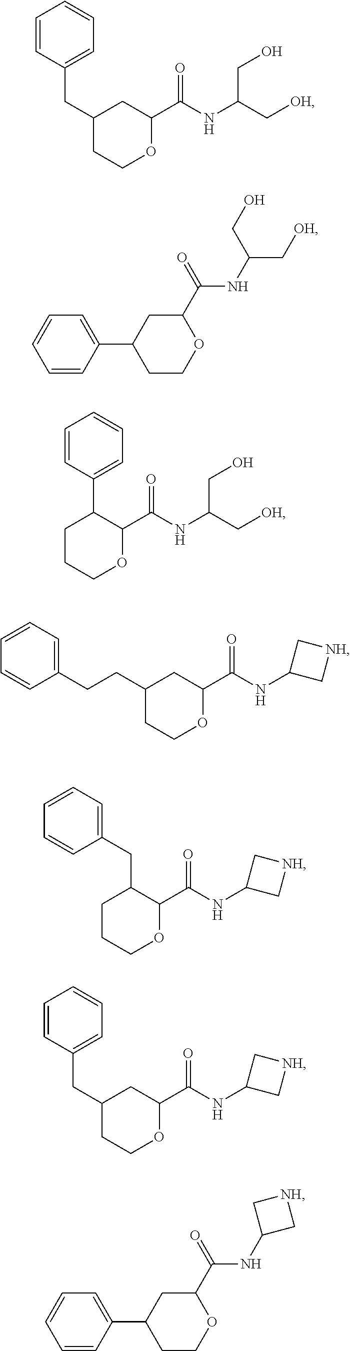

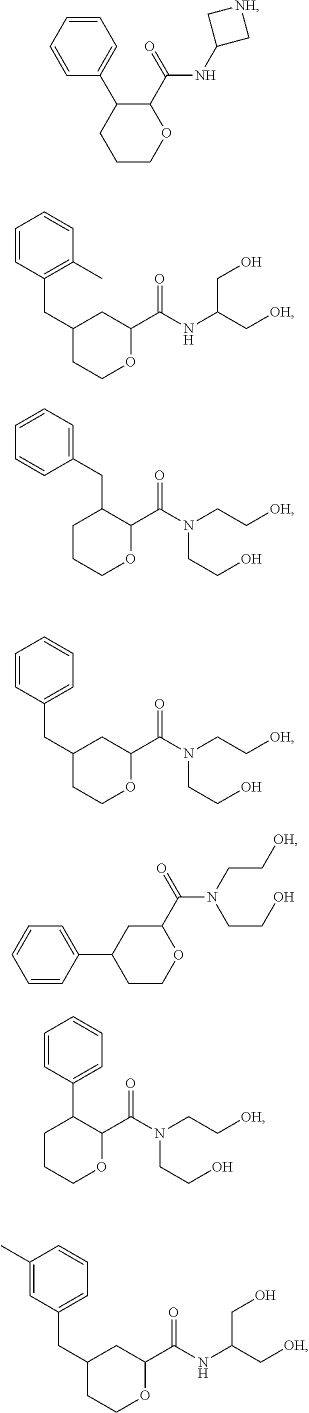

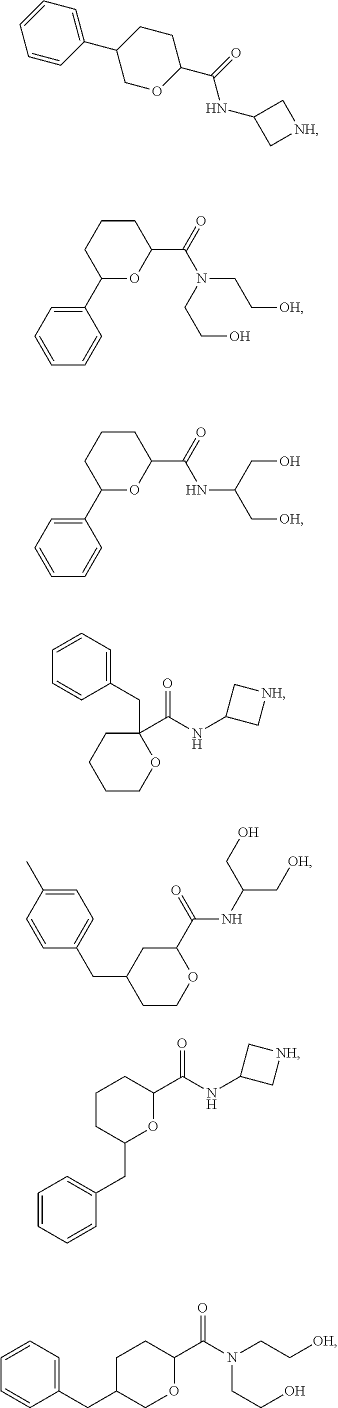

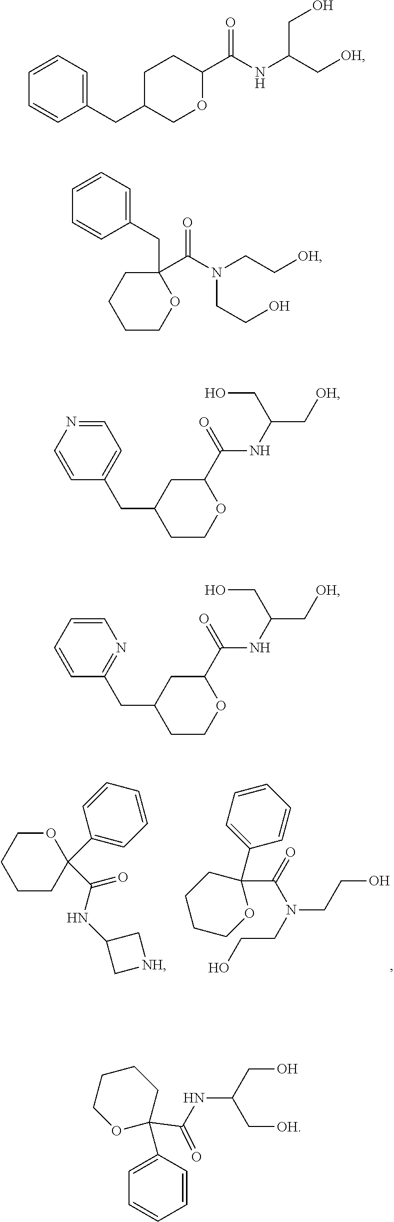

Embodiment 401. A compound according to Formula IV

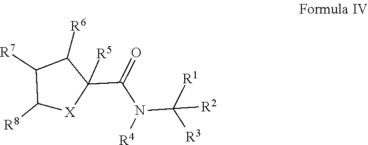

or a pharmaceutically acceptable salt thereof, wherein:
X is —NR— or —O—; where R is independently selected from hydrogen, carbamate, or others defined in R¹
R¹, R², R³ and R⁴ are independently selected from the group consisting of hydrogen, substituted or unsubstituted (C₁-C₁₀) alkyl, substituted or unsubstituted (C₁-C₁₀) alkylene, substituted or unsubstituted (C₁-C₁₀) alkenyl, substituted or unsubstituted (C₁-C₁₀) alkynyl, substituted or unsubstituted (C₁-C₁₀) heteroalkyl, hydroxy (C₁-C₁₀) alkyl, amino (C₁-C₁₀) alkyl, (C₁-C₁₀) alkoxy, (C₁-C₁₀) alkoxy (C₁-C₁₀) alkyl, substituted or unsubstituted cycloalkyl, substituted or unsubstituted heterocyclyl, substituted or unsubstituted aryl, and substituted or unsubstituted heteroaryl; or at least two of any of R¹, R² and R³ taken together form a cycloalkyl or heterocyclyl ring;
R¹, R², R³ and R⁴ are independently substituted with one or more groups selected from the group consisting of hydrogen, halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl;
R⁵, R⁶, R⁷, and R⁸ are independently selected from the group consisting of hydrogen, (C₁-C₁₅) alkyl, (C₁-C₁₅) alkoxy, substituted or unsubstituted (C₁-C₁₅) alkyl aryl, substituted or unsubstituted (C₁-C₁₅) alkyl heteroaryl, substituted or unsubstituted cycloalkyl, substituted or unsubstituted heterocyclyl, substituted or unsubstituted aryl, and substituted or unsubstituted heteroaryl.
Embodiment 402. The compound according to embodiment 401, wherein R⁴ is selected from hydrogen and (C₁-C₁₀) alkyl.
Embodiment 403. The compound according to embodiment 401, wherein R⁴ is hydrogen.
Embodiment 404. The compound according to embodiment 401, wherein each of R⁵, R⁶, R⁷, and R⁸ is hydrogen.
Embodiment 405. The compound according to embodiment 401, wherein R⁴ taken together with any one of R¹, R², and R³ form a nitrogen-containing heterocycle.
Embodiment 406. The compound according to embodiment 405, wherein the nitrogen-containing heterocycle is selected from pyrrolidine, piperidine, and piperazine.
Embodiment 407. The compound according to embodiment 401, wherein at least two of R¹, R² and R³ taken together form a cycloalkyl or heterocyclyl ring.
Embodiment 408. The compound according to embodiment 401, wherein at least two of R¹, R² and R³ taken together form a 5-membered cycloalkyl ring, a 6-membered cycloalkyl ring, a 5-membered heterocyclyl ring, or a 6-membered heterocyclyl ring.
Embodiment 409. The compound according to embodiment 401, wherein at least two of R⁵, R⁶, R⁷, and R⁸ taken together form a cycloalkyl (such as a bridged or fused bicyclic cycloalkyl (e.g., R⁵, R⁶, R⁷, and R⁸ taken together form a bridged bicyclic cycloalkyl, wherein R⁵ and R⁸ taken to together for a —CH₂CH₂— “bridge”; or R⁵, R⁶, R⁷, and R⁸ taken together form a bridged bicyclic cycloalkyl, wherein R⁵ and R⁷ taken to together for a —CH₂CH₂— “bridge”)) or heterocyclyl ring.
Embodiment 410. The compound according to embodiment 401, wherein at least one of R¹, R², and R³ is hydrogen.
Embodiment 411. The compound according to embodiment 401, wherein at least two of R¹, R², and R³ are hydrogen.
Embodiment 412. The compound according to embodiment 401, wherein R⁵ and R⁸ taken together form a heterocyclyl ring.
Embodiment 413. The compound according to embodiment 401 wherein X is NH and the compound has a structure chosen from:

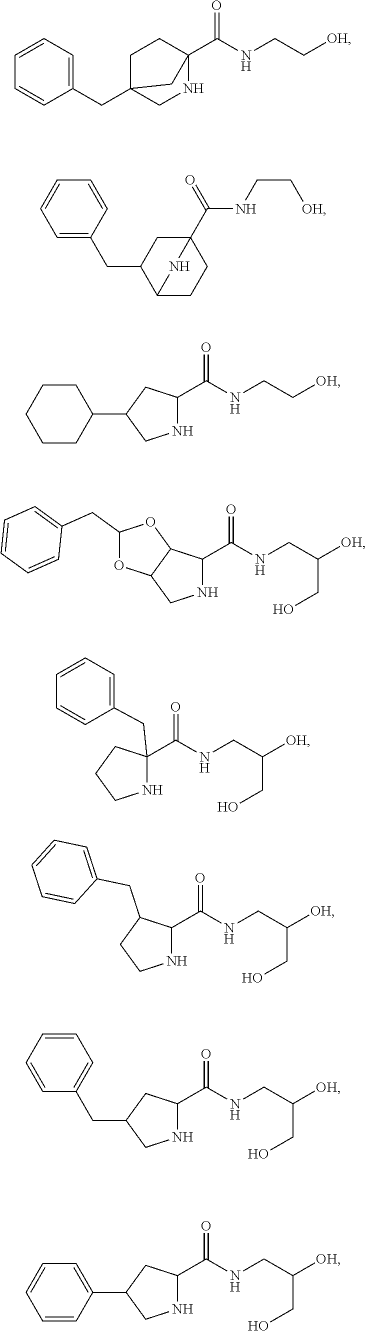

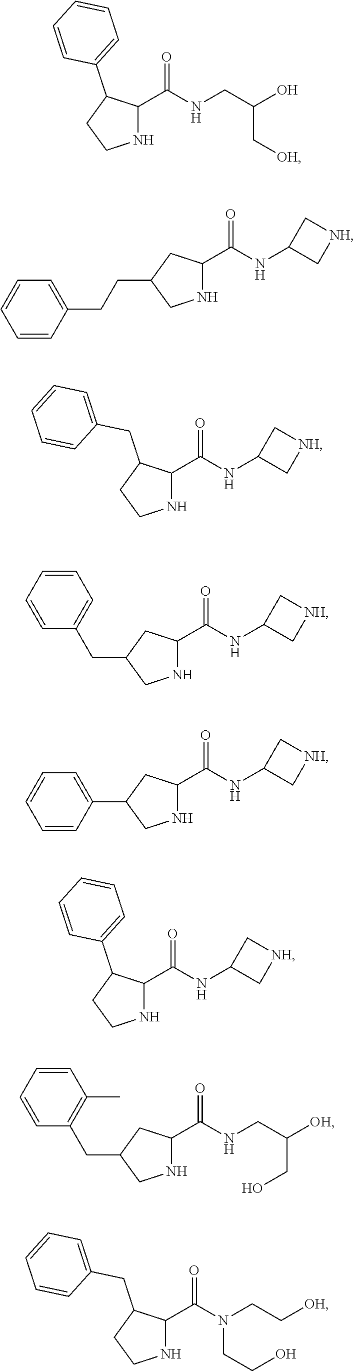

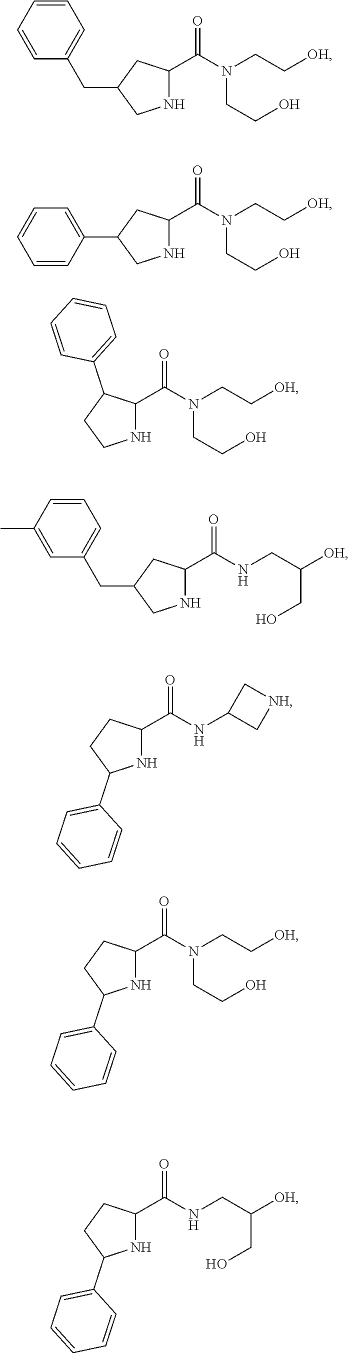

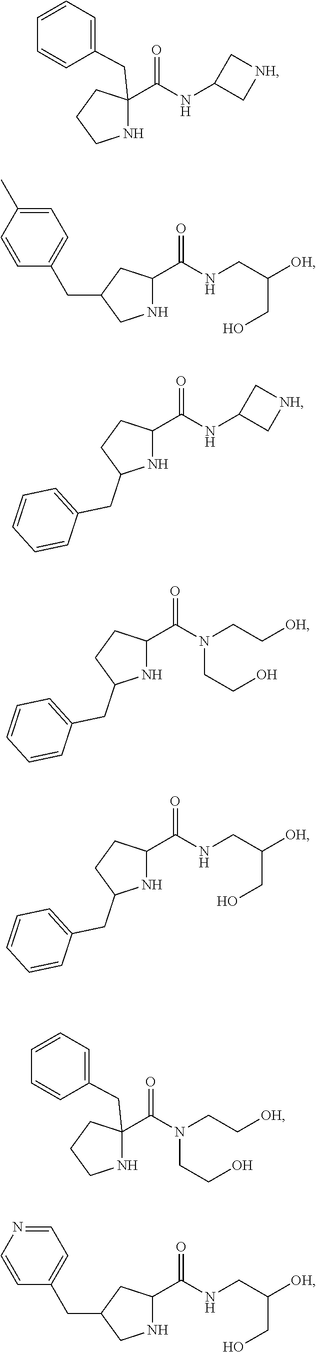

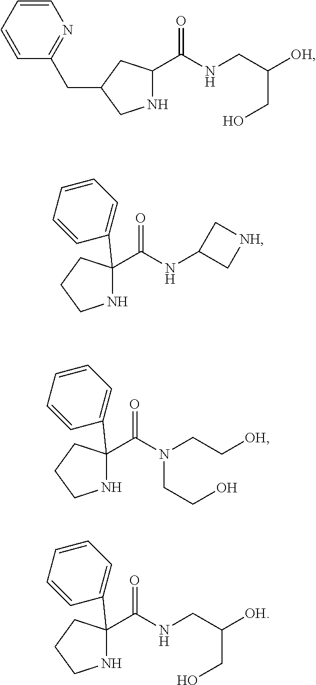

Embodiment 413. The compound according to embodiment 401, wherein X is O and the compound has a structure chosen from:

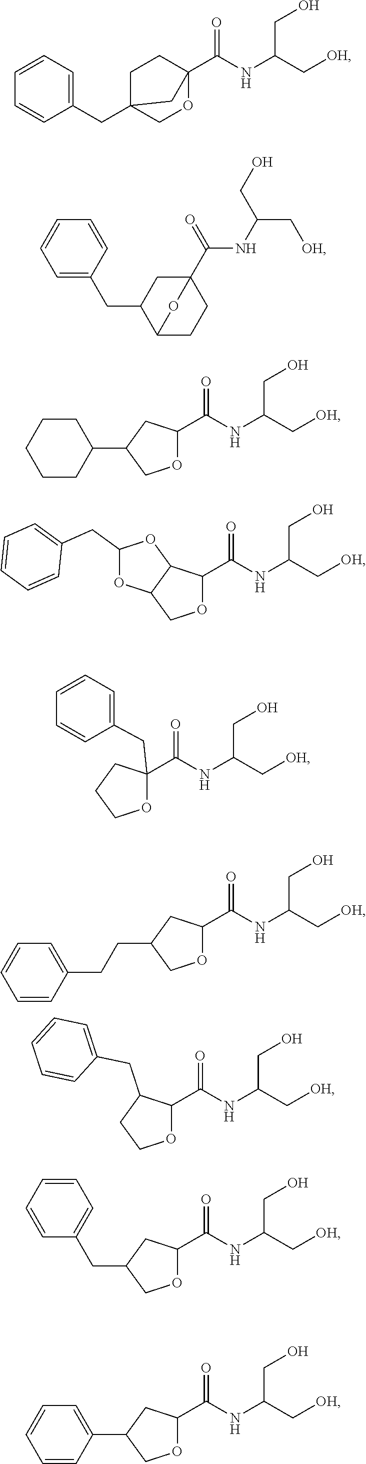

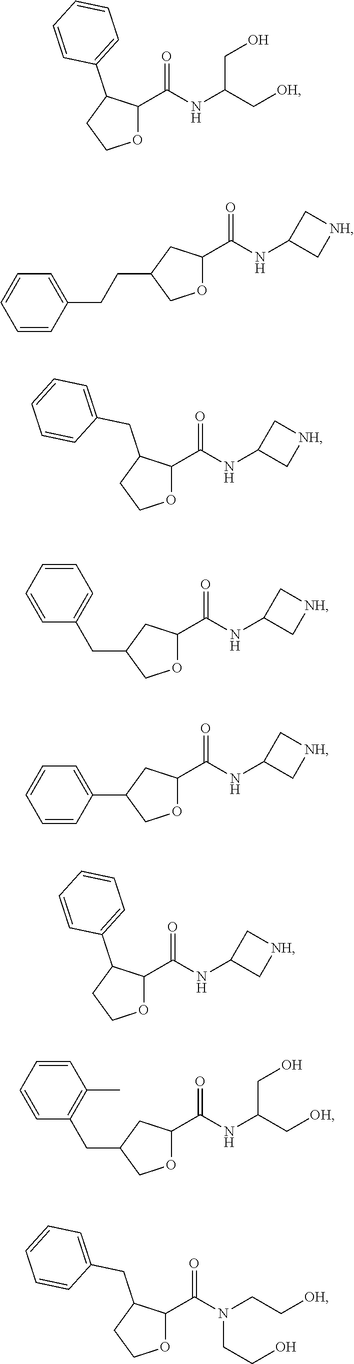

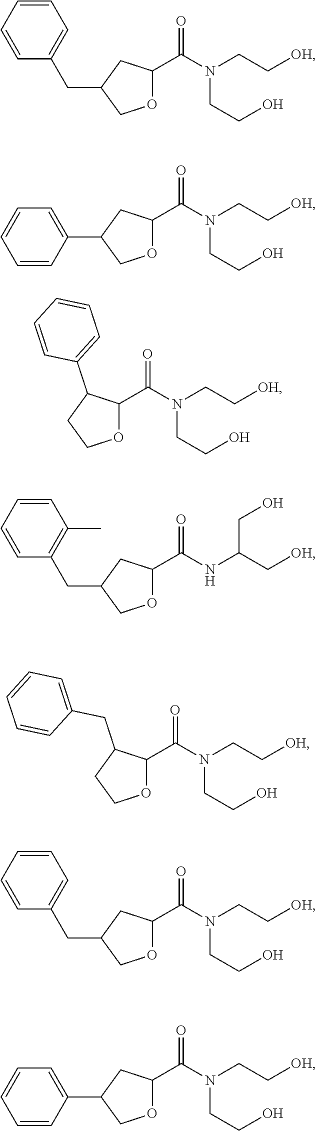

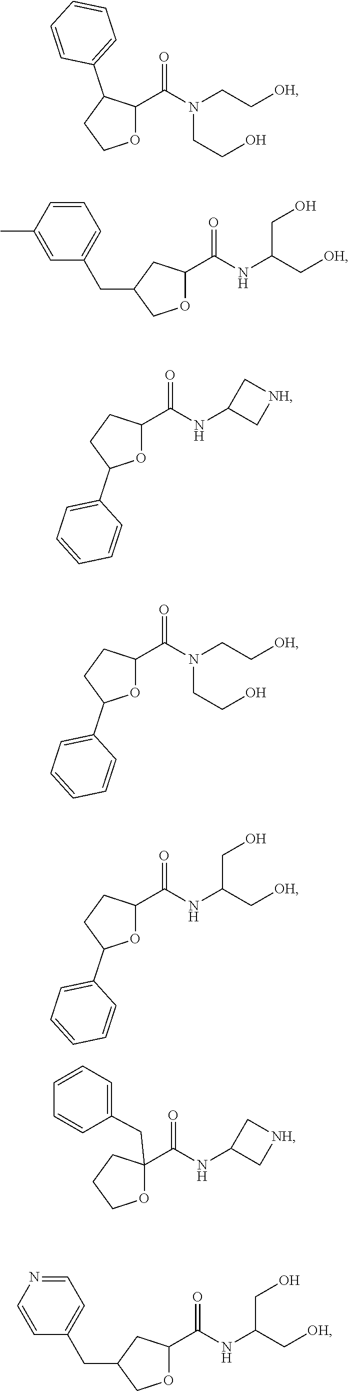

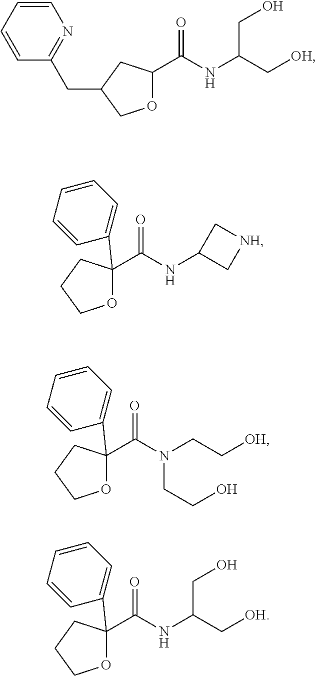

Embodiment 501. A compound according to Formula V

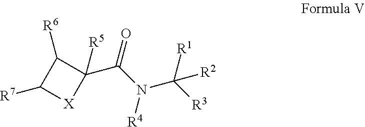

or a pharmaceutically acceptable salt thereof, wherein:
X is —NR— or —O—; wherein R is independently selected from hydrogen, carbamate, or others defined in R¹;
R¹, R², R³ and R⁴ are independently selected from the group consisting of hydrogen, substituted or unsubstituted (C₁-C₁₀) alkyl, substituted or unsubstituted (C₁-C₁₀) alkylene, substituted or unsubstituted (C₁-C₁₀) alkenyl, substituted or unsubstituted (C₁-C₁₀) alkynyl, substituted or unsubstituted (C₁-C₁₀) heteroalkyl, hydroxy (C₁-C₁₀) alkyl, amino (C₁-C₁₀) alkyl, (C₁-C₁₀) alkoxy, (C₁-C₁₀) alkoxy (C₁-C₁₀) alkyl, substituted or unsubstituted cycloalkyl, substituted or unsubstituted heterocyclyl, substituted or unsubstituted aryl, and substituted or unsubstituted heteroaryl; or at least two of any of R¹, R² and R³ taken together form a cycloalkyl or heterocyclyl ring;
R¹, R², R³ and R⁴ are independently substituted with one or more substitutents selected from the group consisting of hydrogen, halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl;
R⁵, R¹ and R⁷ are independently selected from the group consisting of hydrogen, (C₁-C₁₅) alkyl, (C₁-C₁₅) alkoxy, substituted or unsubstituted (C₁-C₁₅) alkyl aryl, substituted or unsubstituted (C₁-C₁₅) alkyl heteroaryl, substituted or unsubstituted cycloalkyl, substituted or unsubstituted heterocyclyl, substituted or unsubstituted aryl, and substituted or unsubstituted heteroaryl.
Embodiment 502. The compound according to embodiment 501, wherein R⁴ is selected from hydrogen and (C₁-C₁₀) alkyl.
Embodiment 503. The compound according to embodiment 501, wherein R⁴ is hydrogen.
Embodiment 504. The compound according to embodiment 501, wherein each of R⁵, R⁶ and R⁷ is hydrogen.
Embodiment 505. The compound according to embodiment 501, wherein R⁴ taken together with any one of R¹, R², and R³ together form a nitrogen-containing heterocycle.
Embodiment 506. The compound according to embodiment 505, wherein the nitrogen-containing heterocycle is selected from pyrrolidine, piperidine, and piperazine.
Embodiment 507. The compound according to embodiment 501, wherein at least two of R¹, R² and R³ taken together form a cycloalkyl or heterocyclyl ring.
Embodiment 508. The compound according to embodiment 501, wherein at least two of R¹, R² and R³ taken together form a 5-membered cycloalkyl ring, a 6-membered cycloalkyl ring, a 5-membered heterocyclyl ring, or a 6-membered heterocyclyl ring.
Embodiment 509. The compound according to embodiment 501, wherein at least two of R⁵, R and R⁷ taken together form a cycloalkyl or heterocyclyl ring.
Embodiment 510. The compound according to embodiment 501, wherein at least one of R¹, R², and R³ is hydrogen.
Embodiment 511. The compound according to embodiment 501, wherein at least two of R¹, R², and R³ are hydrogen.
Embodiment 512. The compound according to embodiment 501, wherein X is NH and the compound has a structure chosen from:

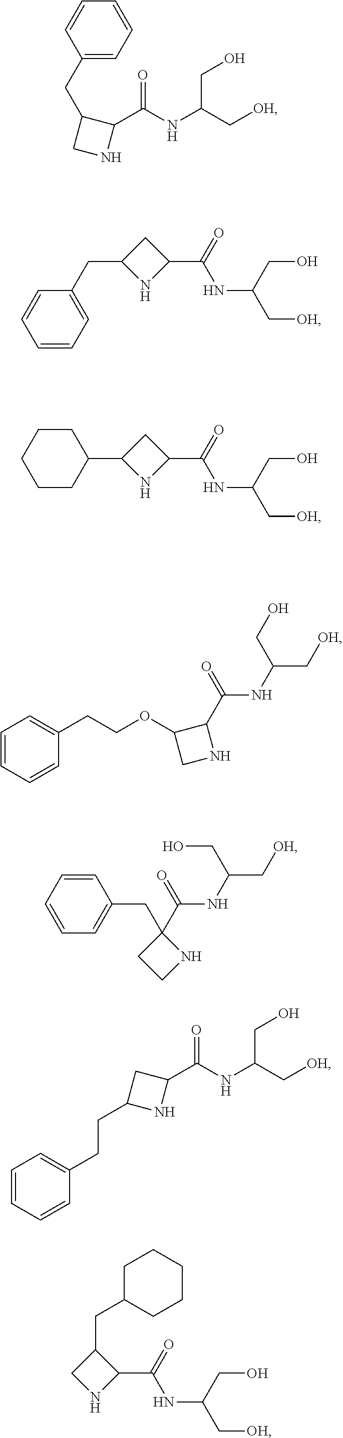

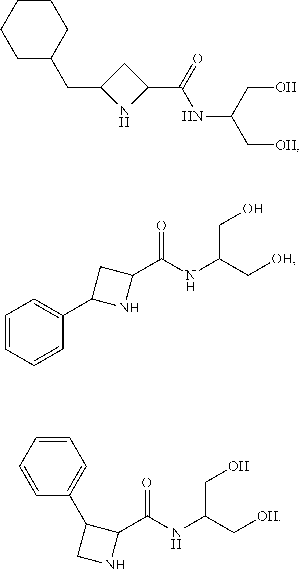 [SMILES: O=C(NC(CO)CO)C1CC(CC2CCCCC2)N1 ; O=C(NC(CO)CO)C1CC(c2ccccc2)N1 ; O=C(NC(CO)CO)C1NCC1c1ccccc1]

Embodiment 513 The compound according to embodiment 501 wherein X is 0 and the compound has a structure selected from:

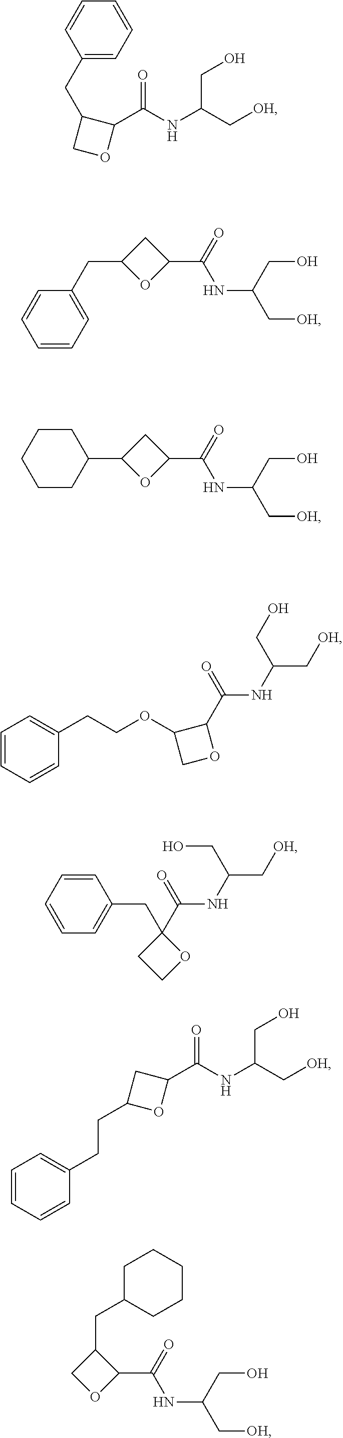

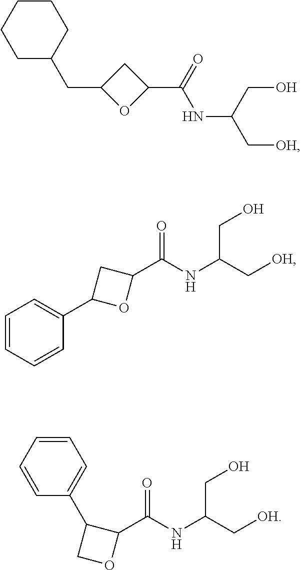 [SMILES: O=C(NC(CO)CO)C1CC(CC2CCCCC2)O1 ; O=C(NC(CO)CO)C1CC(c2ccccc2)O1 ; O=C(NC(CO)CO)C1OCC1c1ccccc1]

Embodiment 601. A compound according to Formula VI

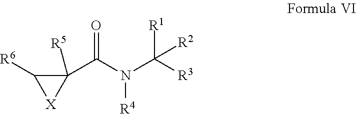

or a pharmaceutically acceptable salt thereof, wherein:
X is —NR— or —O—; wherein R is independently selected from hydrogen, carbamate, or others defined in R¹;
R¹, R², R³ and R⁴ are independently selected from the group consisting of hydrogen, substituted or unsubstituted (C₁-C₁₀) alkyl, substituted or unsubstituted (C₁-C₁₀) alkylene, substituted or unsubstituted (C₁-C₁₀) alkenyl, substituted or unsubstituted (C₁-C₁₀) alkynyl, substituted or unsubstituted (C₁-C₁₀) heteroalkyl, hydroxy (C₁-C₁₀) alkyl, amino (C₁-C₁₀) alkyl, (C₁-C₁₀) alkoxy, (C₁-C₁₀) alkoxy (C₁-C₁₀) alkyl, substituted or unsubstituted cycloalkyl, substituted or unsubstituted heterocyclyl, substituted or unsubstituted aryl, and substituted or unsubstituted heteroaryl; or at least two of any of R¹, R² and R³ taken together form a cycloalkyl or heterocyclyl ring;
R¹, R², R³ and R⁴ are independently substituted with one or more substitutents selected from the group consisting of hydrogen, halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl;
R⁵ and R⁶ are independently selected from the group consisting of hydrogen, (C₁-C₁₅) alkyl, (C₁-C₁₅) alkoxy, substituted or unsubstituted (C₁-C₁₅) alkyl aryl, substituted or unsubstituted (C₁-C₁₅) alkyl heteroaryl, substituted or unsubstituted cycloalkyl, substituted or unsubstituted heterocyclyl, substituted or unsubstituted aryl, and substituted or unsubstituted heteroaryl.
Embodiment 602. The compound according to embodiment 601, wherein R⁴ is selected from hydrogen and (C₁-C₁₀) alkyl.
Embodiment 603. The compound according to embodiment 601, wherein R⁴ is hydrogen.
Embodiment 604. The compound according to embodiment 601, wherein each of R⁵ and R⁶ is hydrogen.
Embodiment 605. The compound according to embodiment 601, wherein R⁴ and any one of R⁵ and R⁶ together form a nitrogen-containing heterocycle.
Embodiment 606. The compound according to embodiment 605, wherein the nitrogen-containing heterocycle is selected from pyrrolidine, piperidine, and piperazine.
Embodiment 607. The compound according to embodiment 601, wherein at least two of R¹, R² and R³ together form a cycloalkyl or heterocyclyl ring.
Embodiment 608. The compound according to embodiment 601, wherein at least two of R¹, R² and R³ taken together form a 5-membered cycloalkyl ring, a 6-membered cycloalkyl ring, a 5-membered heterocyclyl ring, or a 6-membered heterocyclyl ring.
Embodiment 609. The compound according to embodiment 601, wherein R⁵ and R⁶ taken together form a cycloalkyl or heterocyclyl ring.
Embodiment 610. The compound according to embodiment 601, wherein at least one of R¹, R², and R³ is hydrogen.
Embodiment 611. The compound according to embodiment 601, wherein at least two of R¹, R², and R³ are hydrogen.
Embodiment 612. The compound according to embodiment 601, wherein X is NH and the compound has a structure chosen from:

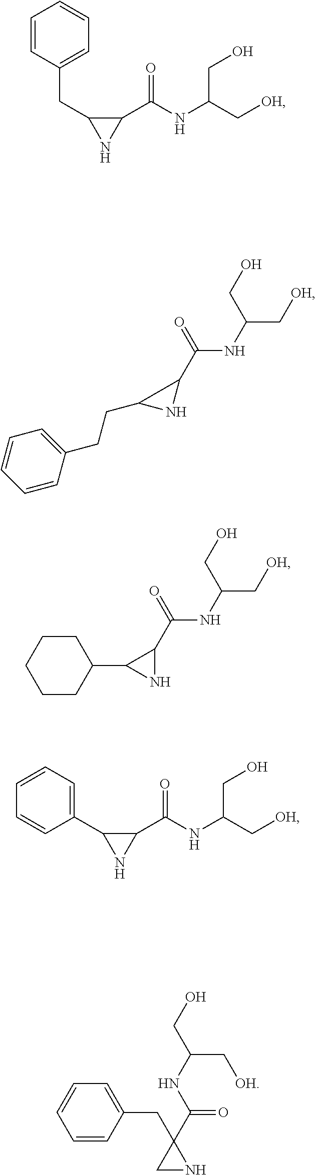 [SMILES: O=C(NC(CO)CO)C1NC1Cc1ccccc1 ; O=C(NC(CO)CO)C1NC1CCc1ccccc1 ; O=C(NC(CO)CO)C1NC1C1CCCCC1 ; O=C(NC(CO)CO)C1NC1c1ccccc1 ; O=C(NC(CO)CO)C1(Cc2ccccc2)CN1]

Embodiment 613. The compound according to embodiment 601, wherein X is O and the compound has a structure selected from:
Embodiment 701. A compound having the structure:

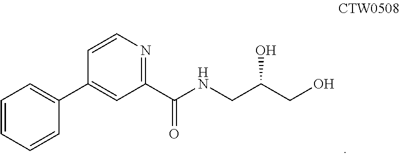 [SMILES: O=C(NC[C@H](O)CO)c1cc(-c2ccccc2)ccn1]

Embodiment 801. A compound having any of the following structures:

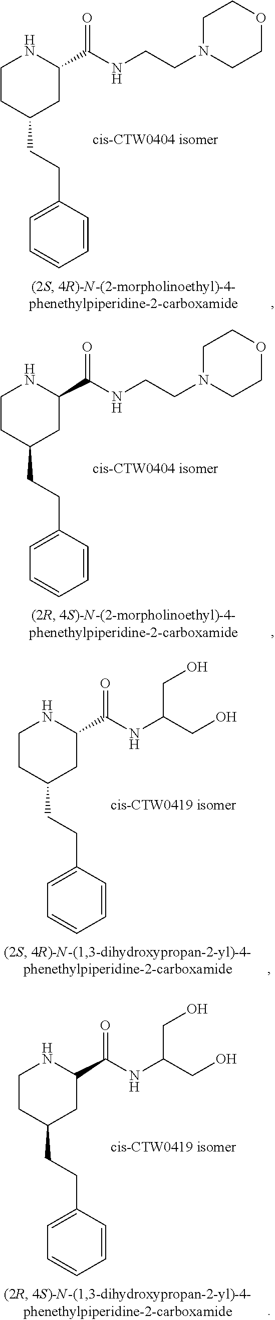

In some embodiments, the invention pertains to racemates or single isomers of compounds with chiral centers disclosed herein including the compounds of Formula I-Formula VI. The stereoisomers may be obtained by known methods such as chiral HPLC separation, stereoselective synthesis, and using optically pure starting reagents.

In certain embodiments, the invention pertains to a compound of any of the following structures:

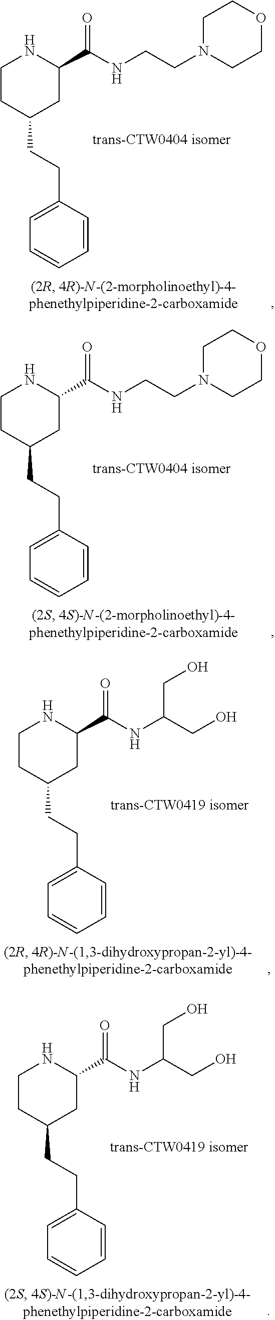 [SMILES: O=C(NCCN1CCOCC1)[C@H]1C[C@H](CCc2ccccc2)CCN1 ; O=C(NCCN1CCOCC1)[C@@H]1C[C@@H](CCc2ccccc2)CCN1 ; O=C(NC(CO)CO)[C@H]1C[C@H](CCc2ccccc2)CCN1 ; O=C(NC(CO)CO)[C@@H]1C[C@@H](CCc2ccccc2)CCN1]

Embodiment 803. A method of separating isomers of modulators disclosed herein using chiral HPLC.
Embodiment 901. A method comprising modulating a 5-hydroxytryptamine 2A receptor and/or a 5-hydroxytryptamine 2C receptor with an effective amount of a compound according to any of the preceding embodiments.

The method according to embodiment 901, wherein the compound modulates the 5-hydroxytryptamine 2A receptor.

The method according to embodiment 901, wherein the compound modulates the 5-hydroxytryptamine 2C receptor.

The method according to embodiment 901, wherein the compound modulates the 5-hydroxytryptamine 2A receptor and the 5-hydroxytryptamine 2C receptor.

The method according to embodiment 901, wherein the compound modulates the 5-hydroxytryptamine 2A receptor to greater extent than the compound modulates the 5-hydroxytryptamine 2C receptor (such that the compound selectly modulates the 5-hydroxytryptamine 2A receptor over 5-hydroxytryptamine 2C receptor).

The method according to embodiment 901, wherein the compound modulates the 5-hydroxytryptamine 2C receptor to greater extent than the compound modulates the 5-hydroxytryptamine 2A receptor.

A non-limiting list of exemplary embodiments of representative compounds according to the present invention is shown in Table 1:

TABLE 1

| | | M.W. | Amount | |
|---|---|---|---|---|
| Compound Code | Structure | (g/mol) | (mg) | Solubility |
| AB0121 | 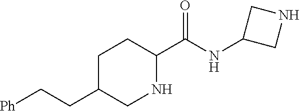 [SMILES: O=C(NC1CNC1)C1CCC(CCc2ccccc2)CN1] | 28701.1998 | 60 | EtOH, DMSO |
| AB0122 | 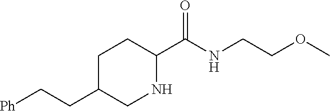 [SMILES: COCCNC(=O)C1CCC(CCc2ccccc2)CN1] | 290.1994 | 50 | EtOH, DMSO |
| AB0123 | 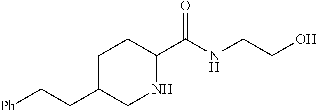 [SMILES: O=C(NCCO)C1CCC(CCc2ccccc2)CN1] | 276.1838 | 15 | EtOH, DMSO |
| AB0124 | 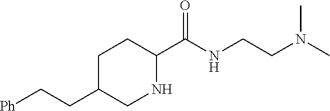 [SMILES: CN(C)CCNC(=O)C1CCC(CCc2ccccc2)CN1] | 303.2311 | 15 | EtOH, DMSO |
| AB0125 | 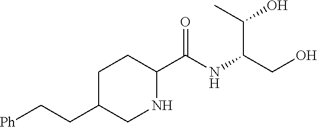 [SMILES: C[C@H](O)[C@H](CO)NC(=O)C1CCC(CCc2ccccc2)CN1] | 320.2100 | 15 | EtOH, DMSO |
| AB0126 | 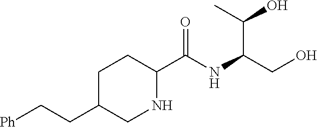 [SMILES: C[C@@H](O)[C@@H](CO)NC(=O)C1CCC(CCc2ccccc2)CN1] | 320.2100 | 16 | EtOH, DMSO |
| AB0127 | 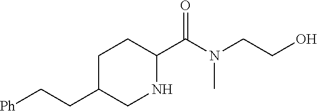 [SMILES: CN(CCO)C(=O)C1CCC(CCc2ccccc2)CN1] | 290.1994 | 23 | EtOH, DMSO |
| AB0128 | 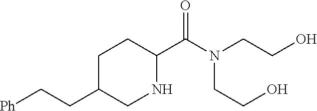 [SMILES: O=C(C1CCC(CCc2ccccc2)CN1)N(CCO)CCO] | 320.2100 | 10 | EtOH, DMSO |
| AB0131 | 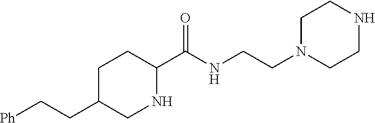 [SMILES: O=C(NCCN1CCNCC1)C1CCC(CCc2ccccc2)CN1] | 344.2576 | 18 | EtOH, DMSO |
| AB0132 | 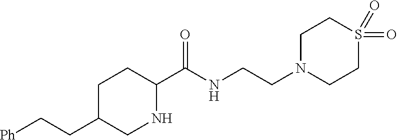 [SMILES: O=C(NCCN1CCS(=O)(=O)CC1)C1CCC(CCc2ccccc2)CN1] | 393.2086 | 18 | EtOH, DMSO |
| AB0135 | 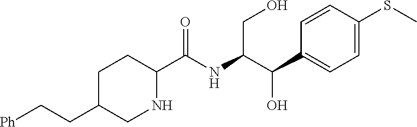 [SMILES: CSc1ccc([C@@H](O)[C@H](CO)NC(=O)C2CCC(CCc3ccccc3)CN2)cc1] | 382.2256 | 30 | EtOH, DMSO |
| AB0136 | 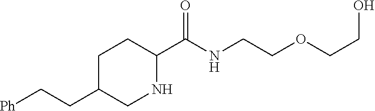 [SMILES: O=C(NCCOCCO)C1CCC(CCc2ccccc2)CN1] | 320.2100 | 25 | EtOH, DMSO |
| AB0145 | 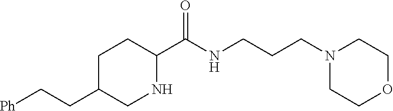 [SMILES: O=C(NCCCN1CCOCC1)C1CCC(CCc2ccccc2)CN1] | 359.2573 | 12 | EtOH, DMSO |
| AB0146 | 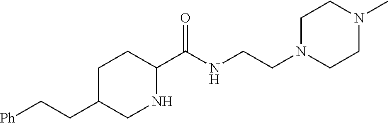 [SMILES: CN1CCN(CCNC(=O)C2CCC(CCc3ccccc3)CN2)CC1] | 358.2733 | 9 | EtOH, DMSO |

Exemplary reaction schemes are provided herein for the preparation of representative compounds according to the present invention.

Exemplary embodiments of piperidines according to Formula I (wherein X is “NH”) and Formula II may be prepared according to reaction Scheme I:

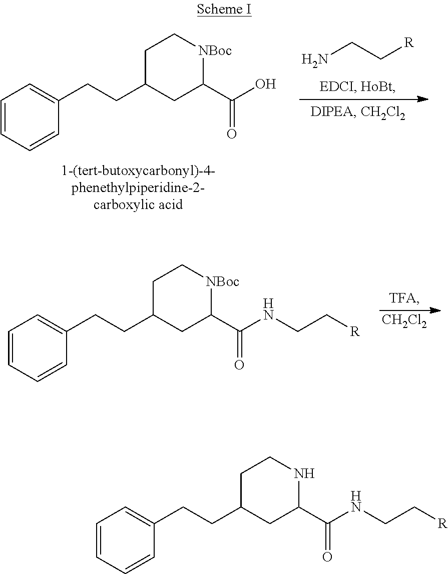

Exemplary embodiments of tetrahydropyran amides according to Formula I (wherein X is “O”) and Formula III may be prepared according to reaction Scheme II:

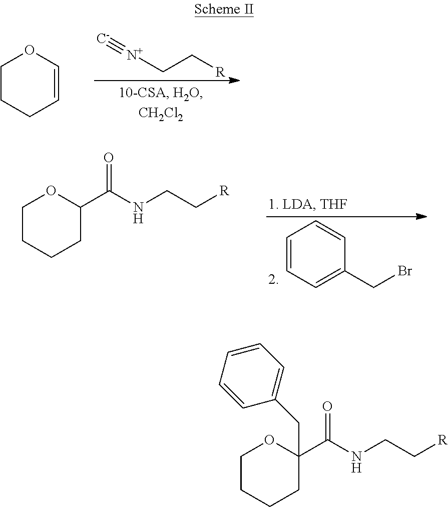

Exemplary embodiments of pyrrolidine amides according to Formula IV wherein X is “NH” may be prepared according to reaction Scheme III:

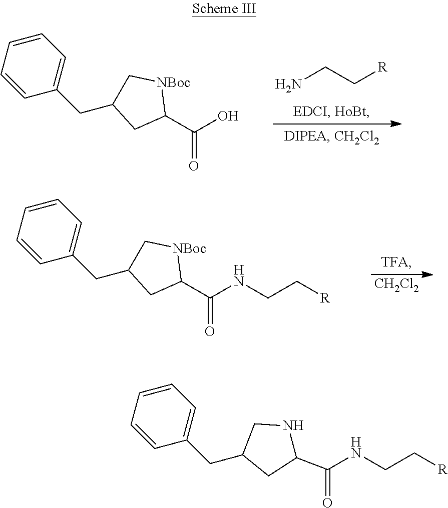

Exemplary embodiments of substituted tetrahydrofuran amides according to Formula IV wherein X is “O” may be prepared according to reaction Scheme IV:

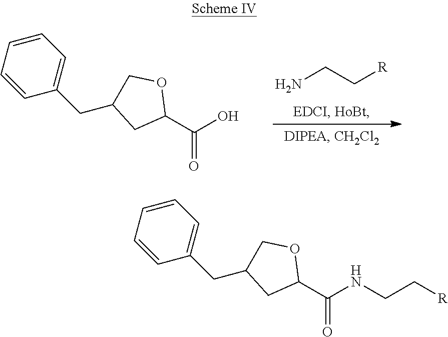

Exemplary embodiments of substituted azetidine amides according to Formula V wherein X is “NH” may be prepared according to reaction Scheme V:

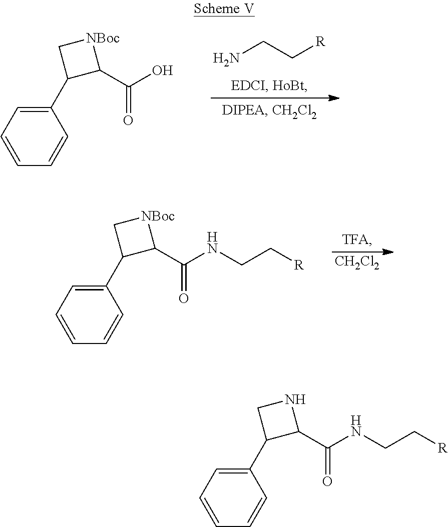

Exemplary embodiments of substituted oxetane amides according to Formula V wherein X is “O” may be prepared according to reaction Scheme VI:

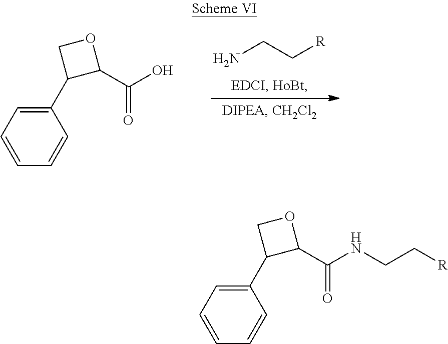

Exemplary embodiments of substituted aziridine carboxamides according to Formula VI wherein X is “NH” may be prepared according to reaction Scheme VII:

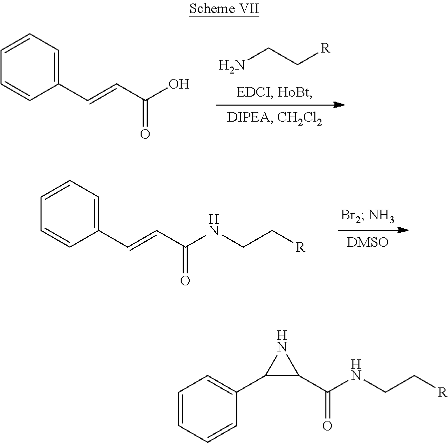

Exemplary embodiments of substituted epoxy amides according to Formula VI wherein X is “O” may be prepared according to reaction Scheme VIII:

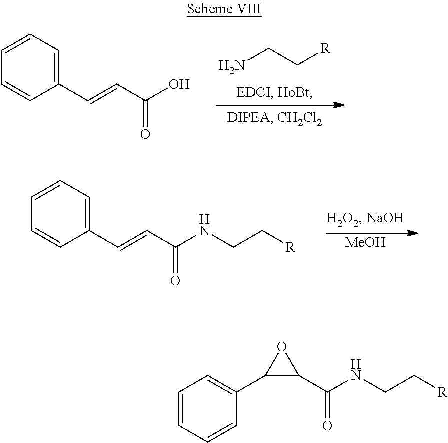

Compounds of CTW0508 may be prepared according to reaction Scheme IX:

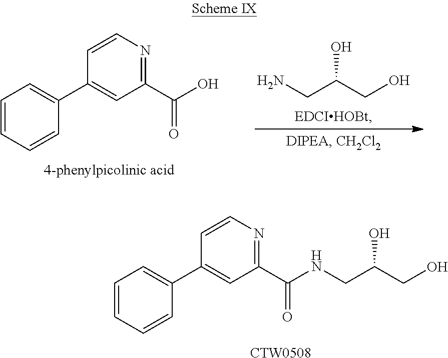 [SMILES: O=C(O)c1cc(-c2ccccc2)ccn1 ; NC[C@H](O)CO ; O=C(NC[C@H](O)CO)c1cc(-c2ccccc2)ccn1 ; "CTW0508"]

It is to be understood that the foregoing descriptions are exemplary, and thus do not restrict the scope of the invention.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Syntheses of exemplary embodiments of carboxamide compounds according to the present invention and their synthetic intermediates are described herein.

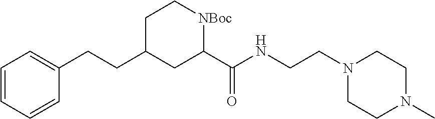 [SMILES: CN1CCN(CCNC(=O)C2CC(CCc3ccccc3)CCN2C(=O)OC(C)(C)C)CC1]

tert-Butyl 2-((2-(4-methylpiperazin-1-yl)ethyl)carbamoyl)-4-phenethylpiperidine-1-carboxylate (AB0111). The synthesis of compound AB0111 was conducted by dissolving 1-(tert-butoxycarbonyl)-4-phenethylpiperidine-2-carboxylic acid, EDCI and HOBt in CH₂Cl₂ at 0° C. under N₂ gas. DIPEA was added and after 1 min 2-(4-methylpiperazin-1-yl)ethan-1-amine was added and reaction was left to reach room temperature overnight. After 18 h reaction was quenched with distilled water, extracted with CH₂Cl₂ and concentration to a colorless oil. Oil was purified by PTLC developed to afford a colorless gel. Yield 44 mg, 72%. ¹H NMR (300 MHz, CDCl₃) δ 7.36-7.11 (m, 6H), 6.59 (t, J=4.7 Hz, 1H), 4.37 (t, J=6.8 Hz, 1H), 3.78 (dt, J=13.6, 5.3 Hz, 1H), 3.35 (p, J=6.1 Hz, 2H), 3.19 (ddd, J=14.0, 10.1, 4.6 Hz, 1H), 2.79-2.32 (m, 13H), 2.27 (d, J=12.1 Hz, 6H), 1.99 (dd, J=7.2, 4.6 Hz, 2H), 1.92-1.74 (m, 1H), 1.63 (td, J=10.3, 5.8 Hz, 4H), 1.47 (s, 10H), 1.42-1.22 (m, 3H). ¹³C NMR (75 MHz, CDCl₃) δ 172.2, 155.7, 142.3, 128.3, 125.7, 80.5, 77.2, 56.3, 55.7, 55.0, 52.7, 45.9, 38.9, 36.4, 35.9, 33.4, 30.3, 29.9, 29.2, 28.4.

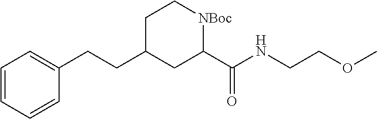 [SMILES: COCCNC(=O)C1CC(CCc2ccccc2)CCN1C(=O)OC(C)(C)C]

tert-Butyl 2-((2-methoxyethyl)carbamoyl)-4-phenethylpiperidine-1-carboxylate (AB0118). The synthesis of compound AB0118 was conducted by following a procedure similar to that of compound AB0111. Yield 40 mg as a colorless gel, 77%. ¹H NMR (300 MHz, CDCl₃) δ 7.22 (dd, J=26.0, 7.6 Hz, 6H), 6.44 (s, 1H), 4.33 (t, J=6.9 Hz, 1H), 3.72 (d, J=30.5 Hz, 1H), 3.48 (s, 4H), 3.31 (s, 3H), 3.18 (s, 1H), 2.73 (s, 2H), 2.03 (s, 4H), 1.71 (s, 3H), 1.47 (s, 10H). ¹³C NMR (75 MHz, CDCl3) δ 172.3, 155.7, 142.2, 128.3, 128.3, 128.2, 125.7, 125.6, 80.6, 77.4, 77.2, 77.0, 76.6, 71.1, 58.6, 56.1, 39.1, 36.5, 33.3, 30.4, 30.0, 29.6, 29.2, 28.3.

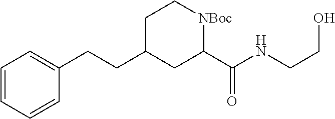 [SMILES: CC(C)(C)OC(=O)N1CCC(CCc2ccccc2)CC1C(=O)NCCO]

tert-Butyl 2-((2-hydroxyethyl)carbamoyl)-4-phenethylpiperidine-1-carboxylate (AB0112). The synthesis of compound AB0112 was conducted by following a procedure similar to that of compound AB0111. Yield 34 mg as a colorless gel, 68%. ¹H NMR (300 MHz, CDCl3) δ 7.23 (dt, J=29.0, 7.6 Hz, 6H), 6.48 (t, J=6.0 Hz, 1H), 4.18 (dd, J=8.5, 6.1 Hz, 1H), 3.70 (t, J=5.1 Hz, 2H), 3.64-3.51 (m, 1H), 3.39 (dtt, J=13.4, 8.7, 3.4 Hz, 3H), 2.66 (p, J=8.4, 7.1 Hz, 3H), 2.04 (ddd, J=9.4, 5.9, 2.7 Hz, 1H), 1.84 (ddd, J=13.7, 8.1, 4.6 Hz, 2H), 1.62 (ddt, J=15.3, 11.6, 6.5 Hz, 4H), 1.46 (s, 10H), 1.29 (dq, J=14.3, 8.7, 7.0 Hz, 3H). ¹³C NMR (75 MHz, CDCl3) δ 173.4, 156.2, 142.1, 128.3, 128.3, 125.7, 81.0, 77.4, 77.2, 77.0, 76.6, 61.7, 57.0, 42.3, 40.4, 36.9, 33.2, 30.9, 30.8, 29.6, 29.5, 28.3.

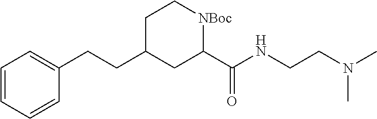 [SMILES: CN(C)CCNC(=O)C1CC(CCc2ccccc2)CCN1C(=O)OC(C)(C)C]

tert-Butyl 2-((2-(dimethylamino)ethyl)carbamoyl)-4-phenethylpiperidine-1-carboxylate (AB0113). The synthesis of compound AB0113 was conducted by following a procedure similar to that of compound AB0111. Yield 28 mg as a colorless gel, 51%. ¹H NMR (300 MHz, CDCl3) δ 7.34-7.10 (m, 6H), 6.57 (s, 1H), 4.32 (t, J=6.9 Hz, 1H), 3.71 (s, 1H), 3.32 (s, 2H), 3.18 (s, 1H), 2.60 (s, 2H), 2.42 (s, 2H), 2.28 (s, 1H), 2.21 (s, 7H), 2.03 (s, 2H), 1.88 (s, 1H), 1.69 (s, 4H), 1.47 (s, 10H). ¹³C NMR (75 MHz, CDCl3) δ 172.3, 155.7, 142.3, 128.3, 128.3, 125.7, 125.6, 80.4, 77.4, 77.2, 77.0, 76.6, 57.8, 56.0, 45.1, 39.1, 36.8, 36.5, 33.4, 30.4, 30.1, 29.6, 29.1, 28.3.

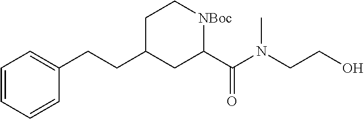 [SMILES: CN(CCO)C(=O)C1CC(CCc2ccccc2)CCN1C(=O)OC(C)(C)C]

tert-Butyl 2-((2-hydroxyethyl)(methyl)carbamoyl)-4-phenethylpiperidine-1-carboxylate (AB0114). The synthesis of compound AB0111 was conducted by following a procedure similar to that of compound AB0114. Yield 36 mg as a colorless gel, 73%. ¹H NMR (300 MHz, CDCl3) δ 7.42-7.05 (m, 5H), 4.07-3.61 (m, 4H), 3.29 (d, J=14.5 Hz, 2H), 3.05 (d, J=39.5 Hz, 4H), 2.64 (q, J=7.2 Hz, 3H), 1.91 (dtd, J=21.2, 11.1, 10.4, 4.8 Hz, 2H), 1.76-1.49 (m, 4H), 1.44 (d, J=2.5 Hz, 9H), 1.30 (q, J=9.1, 7.6 Hz, 3H). ¹³C NMR (75 MHz, CDCl3) δ 173.3, 155.5, 142.1, 128.3, 128.3, 128.2, 125.8, 80.5, 80.4, 77.4, 77.2, 77.0, 76.6, 60.2, 55.3, 52.4, 52.0, 41.0, 37.9, 37.7, 36.8, 33.8, 33.1, 32.9, 31.9, 30.1, 29.6, 29.5, 28.3, 28.3.

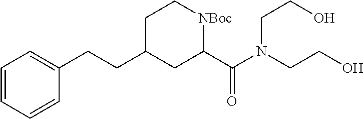 [SMILES: CC(C)(C)OC(=O)N1CCC(CCc2ccccc2)CC1C(=O)N(CCO)CCO]

tert-Butyl 2-(bis(2-hydroxyethyl)carbamoyl)-4-phenethylpiperidine-1-carboxylate (AB0115). The synthesis of compound AB0115 was conducted by following a procedure similar to that of compound AB0111. Yield 20 mg as a colorless gel, 35%. ¹H NMR (300 MHz, CDCl3) δ 7.44-6.92 (m, 5H), 4.51-2.77 (m, 11H), 2.77-2.50 (m, 2H), 2.20-1.53 (m, 6H), 1.45 (d, J=2.9 Hz, 7H), 1.38-0.67 (m, 6H). ¹³C NMR (75 MHz, CDCl3) δ 174.1, 173.9, 172.5, 156.6, 142.5, 142.0, 141.9, 128.4, 128.3, 128.3, 128.2, 125.9, 125.8, 125.6, 80.9, 79.5, 77.4, 77.2, 77.0, 76.6, 61.0, 60.4, 54.7, 52.5, 50.9, 50.3, 42.4, 38.0, 33.6, 33.1, 33.1, 32.9, 32.2, 31.9, 31.4, 31.2, 30.2, 29.9, 29.6, 28.4, 28.3, 28.2.

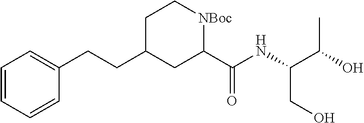 [SMILES: C[C@H](O)[C@H](CO)NC(=O)C1CC(CCc2ccccc2)CCN1C(=O)OC(C)(C)C]

tert-Butyl 2-(((2S,3S)-1,3-dihydroxybutan-2-yl)carbamoyl)-4-phenethylpiperidine-1-carboxylate (AB0116). The synthesis of compound AB0116 was conducted by following a procedure similar to that of compound AB0111. Yield 31 mg as a colorless gel, 54%. ¹H NMR (300 MHz, CDCl3) δ 7.44-7.11 (m, 6H), 6.60 (d, J=8.1 Hz, 1H), 4.17 (ddd, J=14.8, 7.6, 4.1 Hz, 2H), 3.97 (dd, J=11.4, 3.6 Hz, 1H), 3.74 (ddd, J=17.9, 8.4, 3.3 Hz, 2H), 3.49 (q, J=5.5 Hz, 2H), 2.86-2.52 (m, 4H), 2.22-2.00 (m, 2H), 1.81 (ddt, J=22.6, 13.8, 7.7 Hz, 3H), 1.76-1.55 (m, 4H), 1.47 (s, 10H), 1.43-1.06 (m, 9H). ¹³C NMR (75 MHz, CDCl3) δ 173.2, 172.8, 156.8, 156.4, 142.1, 142.1, 128.3, 128.3, 125.8, 125.7, 81.2, 81.1, 77.4, 77.2, 77.0, 76.6, 69.1, 69.0, 64.4, 64.3, 57.7, 55.2, 55.2, 41.2, 37.1, 37.0, 33.1, 33.1, 31.7, 31.3, 31.2, 29.8, 29.6, 28.3, 20.3, 20.3.

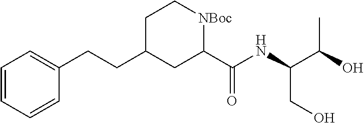 [SMILES: C[C@@H](O)[C@@H](CO)NC(=O)C1CC(CCc2ccccc2)CCN1C(=O)OC(C)(C)C]

tert-Butyl 2-(((2R,3R)-1,3-dihydroxybutan-2-yl)carbamoyl)-4-phenethylpiperidine-1-carboxylate (AB0117). The synthesis of compound AB0117 was conducted by following a procedure similar to that of compound AB0111. Yield 22 mg as a colorless gel, 38%. ¹H NMR (300 MHz, CDCl3) δ 7.44-7.05 (m, 6H), 6.60 (d, J=8.1 Hz, 1H), 4.17 (ddd, J=14.4, 7.6, 4.1 Hz, 2H), 3.95 (dd, J=11.3, 3.6 Hz, 1H), 3.74 (ddt, J=15.5, 7.8, 3.5 Hz, 2H), 3.49 (q, J=7.4 Hz, 2H), 2.65 (q, J=6.8 Hz, 5H), 2.24-1.96 (m, 2H), 1.82 (td, J=14.5, 13.6, 5.2 Hz, 2H), 1.74-1.53 (m, 4H), 1.46 (s, 10H), 1.38-1.04 (m, 8H). ¹³C NMR (75 MHz, CDCl3) δ 173.2, 172.9, 156.8, 156.4, 142.2, 142.1, 128.3, 125.8, 81.2, 81.1, 77.4, 77.2, 77.0, 76.6, 69.0, 68.9, 64.3, 64.2, 57.9, 57.7, 55.2, 55.2, 41.1, 37.1, 37.0, 33.1, 33.1, 31.7, 31.3, 31.2, 29.8, 29.7, 29.6, 28.3, 20.3, 20.3.

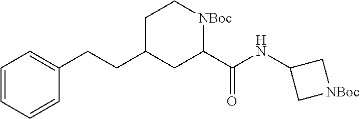 [SMILES: CC(C)(C)OC(=O)N1CC(NC(=O)C2CC(CCc3ccccc3)CCN2C(=O)OC(C)(C)C)C1]

tert-Butyl 2-((1-(tert-butoxycarbonyl)azetidin-3-yl)carbamoyl)-4-phenethylpiperidine-1-carboxylate (AB0119). The synthesis of compound AB0119 was conducted by following a procedure similar to that of compound AB0111. Yield 62 mg as a colorless gel, 95%. ¹H NMR (300 MHz, CDCl3) δ 7.42-7.03 (m, 5H), 6.65 (d, J=7.6 Hz, 1H), 4.62 (dtd, J=12.8, 7.6, 5.2 Hz, 1H), 4.23 (td, J=7.9, 4.3 Hz, 3H), 3.78-3.58 (m, 3H), 3.25 (ddd, J=13.9, 9.4, 4.8 Hz, 1H), 2.63 (q, J=8.5 Hz, 2H), 2.20-1.72 (m, 4H), 1.74-1.51 (m, 3H), 1.45 (d, J=6.9 Hz, 18H). ¹³C NMR (75 MHz, CDCl3) δ 172.3, 156.1, 156.0, 142.1, 128.3, 128.3, 125.7, 80.9, 79.7, 77.5, 77.2, 77.0, 76.6, 56.6, 56.3, 39.9, 39.3, 36.9, 33.2, 30.4, 30.0, 29.6, 29.2, 28.3.

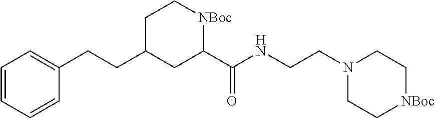 [SMILES: CC(C)(C)OC(=O)N1CCN(CCNC(=O)C2CC(CCc3ccccc3)CCN2C(=O)OC(C)(C)C)CC1]

tert-Butyl 4-(2-(1-(tert-butoxycarbonyl)-4-phenethylpiperidine-2-carboxamido)ethyl)piperazine-1-carboxylate (AB0129). The synthesis of compound AB0129 was conducted by following a procedure similar to that of compound AB0111. Yield 33 mg as a colorless gel, 45%. ¹H NMR (300 MHz, CDCl3) δ 7.31-7.12 (m, 7H), 6.54 (t, J=5.1 Hz, 1H), 4.37 (t, J=6.8 Hz, 1H), 3.77 (dt, J=10.4, 4.9 Hz, 1H), 3.44-3.28 (m, 8H), 3.17 (ddd, J=14.3, 10.3, 4.6 Hz, 2H), 2.62 (s, 1H), 2.47 (t, J=6.0 Hz, 3H), 2.35 (t, J=5.1 Hz, 5H), 2.01 (dd, J=14.1, 7.1 Hz, 3H), 1.86 (s, 1H), 1.61 (qt, J=9.4, 6.4, 5.3 Hz, 5H), 1.36 (dd, J=14.0, 5.1 Hz, 4H), 1.30-1.16 (m, 5H), 0.86 (t, J=8.2 Hz, 2H). ¹³C NMR (75 MHz, CDCl3) δ 172.1, 155.7, 154.6, 142.3, 128.3, 128.3, 125.7, 125.6, 80.5, 79.7, 77.2, 56.4, 55.6, 52.6, 38.8, 36.3, 35.8, 33.4, 30.3, 29.7, 29.6, 29.2, 28.4, 28.3.

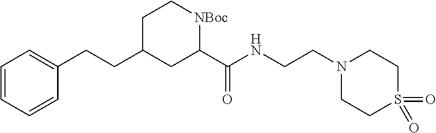 [SMILES: CC(C)(C)OC(=O)N1CCC(CCc2ccccc2)CC1C(=O)NCCN1CCS(=O)(=O)CC1]

tert-Butyl 2-((2-(1,1-dioxidothiomorpholino)ethyl)carbamoyl)-4-phenethylpiperidine-1-carboxylate (AB0130). The synthesis of compound AB0130 was conducted by following a procedure similar to that of compound AB0111. Yield 36 mg as a colorless gel, 54%. ¹H NMR (300 MHz, CDCl3) δ 7.28 (d, J=14.8 Hz, 3H), 7.18 (t, J=5.6 Hz, 3H), 6.42 (d, J=5.8 Hz, 1H), 4.35 (t, J=6.9 Hz, 1H), 3.73 (dt, J=11.2, 5.0 Hz, 1H), 3.36 (p, J=7.1 Hz, 2H), 3.16 (ddd, J=14.0, 10.3, 4.6 Hz, 1H), 3.00 (d, J=10.0 Hz, 8H), 2.64 (dt, J=13.0, 7.4 Hz, 5H), 2.08-1.87 (m, 4H), 1.72-1.54 (m, 4H), 1.46 (s, 9H), 1.28 (q, J=10.6, 7.1 Hz, 5H), 0.87 (d, J=10.2 Hz, 1H). ¹³C NMR (75 MHz, CDCl3) δ 172.3, 155.9, 142.2, 128.3, 125.8, 80.6, 77.4, 77.2, 77.0, 76.6, 60.3, 55.8, 55.2, 51.2, 50.6, 39.2, 36.6, 36.5, 33.4, 30.3, 29.6, 29.6, 29.3, 28.3.

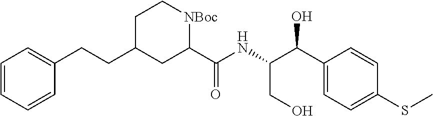 [SMILES: CSc1ccc([C@H](O)[C@H](CO)NC(=O)C2CC(CCc3ccccc3)CCN2C(=O)OC(C)(C)C)cc1]

tert-Butyl 2-(((1S,2S)-1,3-dihydroxy-1-(4-(methylthio)phenyl)propan-2-yl)carbamoyl)-4-phenethylpiperidine-1-carboxylate (AB0133). The synthesis of compound AB0133 was conducted by following a procedure similar to that of compound AB011. Yield 60 mg as a colorless gel, 84%. ¹H NMR (300 MHz, CDCl3) δ 7.34-7.23 (m, 5H), 7.16 (ddd, J=18.7, 8.2, 3.6 Hz, 6H), 6.56 (t, J=7.0 Hz, 1H), 5.04 (dd, J=21.3, 3.7 Hz, 1H), 4.25 (s, 1H), 4.20-4.00 (m, 2H), 4.00-3.68 (m, 4H), 3.51-3.37 (m, 1H), 2.66-2.50 (m, 2H), 2.42 (d, J=11.2 Hz, 4H), 2.05 (s, 1H), 1.95-1.61 (m, 3H), 1.45 (d, J=6.0 Hz, 11H), 1.30 (dt, J=18.9, 7.6 Hz, 6H), 1.16 (s, 1H), 0.88 (dd, J=13.4, 6.1 Hz, 1H). ¹³C NMR (75 MHz, CDCl3) δ 173.1, 173.0, 142.1, 142.1, 138.1, 138.0, 137.7, 137.5, 128.4, 128.3, 128.3, 126.5, 126.4, 126.2, 125.8, 125.7, 81.2, 81.2, 77.4, 77.0, 76.6, 73.4, 73.0, 63.3, 63.0, 60.4, 57.1, 56.7, 56.6, 36.3, 33.1, 33.0, 30.5, 30.4, 29.7, 29.4, 28.3, 15.8, 14.1.

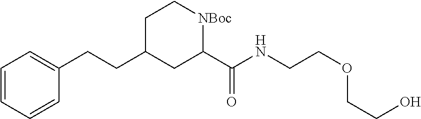 [SMILES: CC(C)(C)OC(=O)N1CCC(CCc2ccccc2)CC1C(=O)NCCOCCO]

tert-Butyl 2-((2-(2-hydroxyethoxy)ethyl)carbamoyl)-4-phenethylpiperidine-1-carboxylate (AB0134). The synthesis of compound AB0134 was conducted by following a procedure similar to that of compound AB0111. Yield 50 mg as a colorless gel, 89%. ¹H NMR (300 MHz, CDCl3) δ 7.33-7.12 (m, 6H), 6.66 (t, J=5.7 Hz, 1H), 4.34 (t, J=7.0 Hz, 1H), 3.73 (dt, J=13.9, 4.9 Hz, 3H), 3.51 (ddd, J=22.6, 7.6, 3.4 Hz, 7H), 3.20 (ddd, J=14.1, 10.2, 4.7 Hz, 1H), 2.74-2.54 (m, 3H), 2.10-2.02 (m, 1H), 1.98 (s, 2H), 1.86 (dq, J=13.4, 5.2 Hz, 1H), 1.62 (h, J=10.1, 9.5 Hz, 4H), 1.46 (s, 10H), 1.43-1.30 (m, 2H), 1.26 (s, 2H). ¹³C NMR (75 MHz, CDCl3) δ 172.4, 156.0, 142.2, 128.3, 128.3, 125.7, 80.7, 77.4, 77.0, 76.6, 72.1, 69.5, 61.6, 55.9, 39.2, 39.0, 36.5, 33.3, 30.2, 29.8, 29.6, 29.1, 28.3.

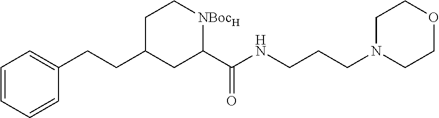

tert-Butyl 2-((3-morpholinopropyl)carbamoyl)-4-phenethylpiperidine-1-carboxylate (AB0143). The synthesis of compound AB0143 was conducted by following a procedure similar to that of compound AB0111. Yield 28 mg as a colorless gel, 45%. ¹H NMR (300 MHz, CDCl3) δ 7.34-7.12 (m, 6H), 6.83 (t, J=5.5 Hz, 1H), 4.25 (t, J=7.1 Hz, 1H), 3.76-3.60 (m, 5H), 3.45-3.19 (m, 3H), 2.65 (ddd, J=14.5, 8.7, 4.6 Hz, 2H), 2.47-2.35 (m, 6H), 2.09-1.95 (m, 2H), 1.88 (dt, J=11.2, 6.1 Hz, 2H), 1.76-1.62 (m, 4H), 1.46 (s, 9H), 1.34 (dd, J=12.7, 5.8 Hz, 2H), 1.27 (s, 2H). ¹³C NMR (75 MHz, CDCl3) δ 172.3, 156.0, 142.2, 128.3, 125.7, 80.6, 77.4, 77.2, 77.0, 76.6, 66.9, 66.8, 57.4, 56.6, 53.8, 39.9, 38.8, 36.7, 33.3, 30.7, 30.4, 29.6, 29.4, 28.3, 25.3.

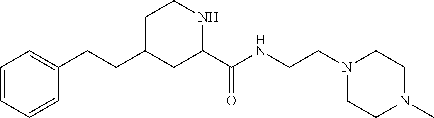 [SMILES: CN1CCN(CCNC(=O)C2CC(CCc3ccccc3)CCN2)CC1]

N-(2-(4-methylpiperazin-1-yl)ethyl)-4-phenethylpiperidine-2-carboxamide (AB0146). The synthesis of compound AB0146 was conducted by following dissolving AB011 in CH₂Cl₂ at 0° C. under N₂ gas. TFA was added dropwise to solution and reaction was allowed to warm to room temperature over 3 hours. Solvent was removed and oil was redesolved in CH₂Cl₂ 3×. a solution of product CH₂Cl₂ was treat with NaHCO₃, extract with CH₂Cl₂ and dried over Na₂SO₄. Removal of solvent gave product with no further purification need. Yield 3.1 mg as a colorless gel, 45%. ¹H NMR (300 MHz, MeOD) δ 7.33-7.09 (m, 5H), 3.47-3.33 (m, 1H), 3.24-3.17 (m, 1H), 3.15 (s, 1H), 2.95-2.57 (m, 1H), 2.52 (t, J=6.7 Hz, 5H), 2.29 (s, 3H), 2.01 (d, J=13.2 Hz, 1H), 1.76 (d, J=13.1 Hz, 1H), 1.59 (dt, J=9.1, 6.3 Hz, 3H), 1.31 (s, 3H), 1.22-0.98 (m, 3H). ¹³C NMR (75 MHz, MeOD) δ 174.5, 142.3, 127.9, 125.3, 59.9, 56.5, 54.3, 52.2, 45.0, 44.5, 38.8, 36.5, 35.7, 35.2, 32.2, 32.0, 29.3.

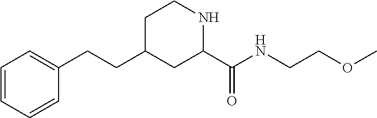 [SMILES: COCCNC(=O)C1CC(CCc2ccccc2)CCN1]

N-(2-methoxyethyl)-4-phenethylpiperidine-2-carboxamide (AB0122). The synthesis of compound AB0122 was conducted by following a procedure similar to that of compound AB0146. Yield 32 mg as a colorless gel, 84%. ¹H NMR (300 MHz, CDCl3) δ 10.10 (s, 3H), 9.58 (s, 1H), 7.74 (s, 1H), 7.56-6.97 (m, 7H), 4.13 (s, 1H), 3.76-3.23 (m, 8H), 3.11 (s, 1H), 2.67 (t, J=7.5 Hz, 2H), 2.18 (d, J=13.6 Hz, 1H), 2.00 (d, J=14.1 Hz, 1H), 1.88-1.38 (m, 5H). ¹³C NMR (75 MHz, CDCl3) δ 168.9, 141.3, 128.5, 128.2, 126.0, 77.2, 70.2, 58.6, 58.2, 43.7, 39.5, 37.5, 33.4, 33.3, 32.5, 28.0.

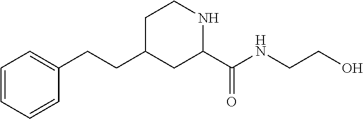 [SMILES: O=C(NCCO)C1CC(CCc2ccccc2)CCN1]

N-(2-hydroxyethyl)-4-phenethylpiperidine-2-carboxamide (AB0123). The synthesis of compound AB0123 was conducted by following a procedure similar to that of compound AB0146. Yield 9.5 mg as a colorless gel, 86%. ¹H NMR (300 MHz, MeOD) δ 7.32-7.09 (m, 4H), 3.46-3.24 (m, 5H), 3.21-3.06 (m, 2H), 2.72-2.53 (m, 3H), 2.47 (t, J=6.7 Hz, 2H), 2.29 (s, 5H), 2.07-1.96 (m, 1H), 1.76 (d, J=13.2 Hz, 1H), 1.66-1.45 (m, 3H), 1.33 (d, J=12.3 Hz, 1H), 1.22-0.98 (m, 2H). ¹³C NMR (75 MHz, CDCl3) δ 141.1, 141.1, 128.5, 128.1, 126.1, 77.2, 65.6, 58.3, 43.9, 38.1, 37.3, 33.4, 33.3, 33.2, 32.4, 32.3, 28.0.

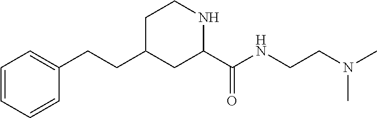 [SMILES: CN(C)CCNC(=O)C1CC(CCc2ccccc2)CCN1]

N-(2-(dimethylamino)ethyl)-4-phenethylpiperidine-2-carboxamide (AB0124). The synthesis of compound AB0124 was conducted by following a procedure similar to that of compound AB0146. Yield 8 mg as a colorless gel, 64%. ¹H NMR (300 MHz, MeOD) δ 7.32-7.22 (m, 2H), 7.22-7.09 (m, 3H), 3.62 (t, J=5.7 Hz, 2H), 3.33 (d, J=11.4 Hz, 4H), 3.25-3.07 (m, 2H), 2.72-2.53 (m, 3H), 2.02 (dq, J=12.6, 2.9 Hz, 1H), 1.76 (dd, J=12.9, 2.9 Hz, 1H), 1.68-1.42 (m, 3H), 1.31 (s, 1H), 1.25-1.00 (m, 2H). ¹³C NMR (75 MHz, CDCl3) δ 141.1, 128.5, 128.2, 126.1, 77.2, 59.5, 58.0, 44.8, 43.9, 43.8, 37.3, 34.4, 33.4, 32.5, 32.3, 29.7, 28.2.

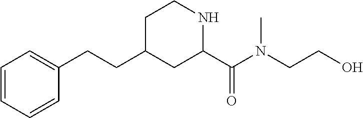 [SMILES: CN(CCO)C(=O)C1CC(CCc2ccccc2)CCN1]

N-(2-hydroxyethyl)-N-methyl-4-phenethylpiperidine-2-carboxamide (AB0127). The synthesis of compound AB0127 was conducted by following a procedure similar to that of compound AB0146. Yield 17 mg as a colorless gel, 100%. ¹H NMR (300 MHz, MeOD) δ 7.33-7.09 (m, 5H), 3.78-3.62 (m, 3H), 3.59-3.40 (m, 2H), 3.22-3.04 (m, 2H), 2.97 (s, 1H), 2.73-2.54 (m, 3H), 1.93 (d, J=13.1 Hz, 1H), 1.77 (d, J=12.8 Hz, 1H), 1.58 (ddt, J=8.4, 5.3, 2.9 Hz, 3H), 1.31 (s, 1H), 1.21-0.97 (m, 2H). ¹³C NMR (75 MHz, CDCl3) δ 173.3, 155.5, 142.1, 128.3, 128.3, 125.8, 80.5, 80.4, 77.2, 60.2, 55.3, 52.4, 52.0, 41.0, 37.9, 37.7, 36.7, 33.8, 33.1, 32.9, 31.9, 31.9, 30.1, 29.6, 29.5, 28.3.

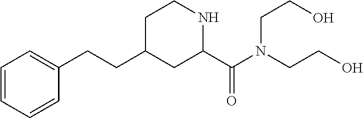 [SMILES: O=C(C1CC(CCc2ccccc2)CCN1)N(CCO)CCO]

N,N-bis(2-hydroxyethyl)-4-phenethylpiperidine-2-carboxamide (AB0128). The synthesis of compound AB0128 was conducted by following a procedure similar to that of compound AB0146. Yield 7.2 mg as a colorless gel, 95%. ¹H NMR (300 MHz, MeOD) δ 7.33-7.10 (m, 5H), 3.71 (tdd, J=7.8, 6.8, 4.6 Hz, 4H), 3.58 (q, J=6.0 Hz, 2H), 3.45 (dt, J=13.8, 5.9 Hz, 1H), 2.80-2.55 (m, 4H), 1.95 (d, J=13.2 Hz, 1H), 1.77 (d, J=12.8 Hz, 1H), 1.58 (t, J=5.8 Hz, 3H), 1.32 (d, J=3.7 Hz, 3H), 1.14 (s, 1H), 1.10 (s, 1H), 0.91 (d, J=5.8 Hz, 1H). ¹³C NMR (75 MHz, CDCl3) δ 174.2, 142.0, 128.4, 128.3, 128.3, 125.8, 125.8, 81.0, 61.0, 60.4, 54.7, 52.5, 50.9, 38.0, 32.9, 32.1, 31.9, 29.9, 29.6, 28.3, 28.2.

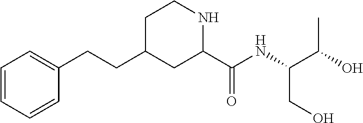 [SMILES: C[C@H](O)[C@H](CO)NC(=O)C1CC(CCc2ccccc2)CCN1]

N-((2S,3S)-1,3-dihydroxybutan-2-yl)-4-phenethylpiperidine-2-carboxamide (AB0125). The synthesis of compound AB0125 was conducted by following a procedure similar to that of compound AB0146. Yield 10 mg as a colorless gel, 910%. ¹H NMR (300 MHz, MeOD) δ 7.32-7.09 (m, 5H), 4.03 (qt, J=6.4, 3.2 Hz, 1H), 3.82 (tt, J=6.2, 3.2 Hz, 1H), 3.63 (dtd, J=12.8, 11.1, 6.2 Hz, 2H), 3.18 (dddd, J=28.0, 12.6, 4.7, 2.6 Hz, 2H), 2.73-2.54 (m, 3H), 2.06 (d, J=12.4 Hz, 1H), 1.76 (d, J=12.8 Hz, 1H), 1.66-1.50 (m, 3H), 1.48-1.29 (m, 2H), 1.25-1.00 (m, 6H), 0.95-0.87 (m, 1H). ¹³C NMR (75 MHz, MeOD) δ 168.9, 141.7, 128.0, 127.9, 125.5, 65.6, 61.3, 58.0, 56.4, 48.4, 48.1, 47.8, 47.8, 47.7, 47.7, 47.5, 47.5, 47.4, 47.3, 47.2, 47.2, 47.0, 46.7, 46.7, 46.7, 43.2, 37.5, 33.6, 33.4, 32.1, 27.7, 19.0.

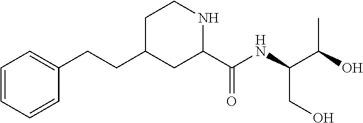 [SMILES: C[C@@H](O)[C@@H](CO)NC(=O)C1CC(CCc2ccccc2)CCN1]

N-((2R,3R)-1,3-dihydroxybutan-2-yl)-4-phenethylpiperidine-2-carboxamide (AB0126). The synthesis of compound AB0126 was conducted by following a procedure similar to that of compound AB0146. Yield 11.6 mg as a colorless gel, 98%. ¹H NMR (300 MHz, MeOD) δ 7.40-6.99 (m, 5H), 4.03 (dp, J=6.4, 3.2 Hz, 1H), 3.82 (tt, J=6.1, 3.2 Hz, 1H), 3.74-3.52 (m, 2H), 3.33 (p, J=1.6 Hz, 3H), 3.23 (ddd, J=11.6, 5.1, 2.8 Hz, 1H), 3.14 (ddd, J=12.6, 4.2, 2.3 Hz, 1H), 2.73-2.55 (m, 3H), 2.12-1.99 (m, 1H), 1.76 (dt, J=12.8, 2.7 Hz, 1H), 1.66-1.49 (m, 3H), 1.43-1.24 (m, 2H), 1.17 (d, J=6.5 Hz, 4H), 1.14-1.00 (m, 2H). ¹³C NMR (75 MHz, MeOD) δ 168.9, 141.7, 141.7, 128.0, 127.9, 125.5, 65.6, 65.5, 61.4, 61.3, 58.1, 58.0, 56.4, 56.2, 48.4, 48.1, 48.1, 47.8, 47.8, 47.7, 47.6, 47.5, 47.4, 47.3, 47.2, 47.0, 46.7, 43.2, 37.5, 37.4, 33.7, 33.6, 33.5, 33.4, 32.1, 27.7, 19.2, 19.0.

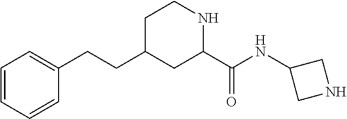 [SMILES: O=C(NC1CNC1)C1CC(CCc2ccccc2)CCN1]

N-(azetidin-3-yl)-4-phenethylpiperidine-2-carboxamide (AB0121). The synthesis of compound AB0121 was conducted by following a procedure similar to that of compound AB0146. Yield 35 mg as a colorless gel, 100%. ¹H NMR (300 MHz, MeOD) δ 7.38-7.09 (m, 5H), 4.84-4.65 (m, 1H), 4.40-4.11 (m, 4H), 3.87 (dd, J=12.7, 3.2 Hz, 1H), 3.45 (ddd, J=12.8, 4.4, 2.1 Hz, 1H), 3.04 (td, J=13.0, 3.1 Hz, 1H), 2.70 (dd, J=8.9, 6.6 Hz, 2H), 2.41-2.28 (m, 1H), 2.02 (d, J=14.2 Hz, 1H), 1.85-1.60 (m, 3H), 1.54-1.25 (m, 3H). ¹³C NMR (75 MHz, MeOD) δ 168.8, 141.6, 128.0, 127.9, 125.5, 57.6, 52.0, 51.9, 43.3, 42.0, 37.5, 33.4, 33.0, 32.1, 27.7.

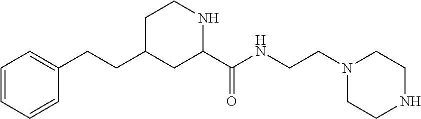 [SMILES: O=C(NCCN1CCNCC1)C1CC(CCc2ccccc2)CCN1]

4-phenethyl-N-(2-(piperazin-1-yl)ethyl)piperidine-2-carboxamide (AB0131). The synthesis of compound AB0131 was conducted by following a procedure similar to that of compound AB0146. Yield 11 mg as a colorless gel, 100%. ¹H NMR (300 MHz, MeOD) δ 7.33-7.09 (m, 5H), 3.37 (dt, J=6.8, 3.5 Hz, 5H), 3.22-3.06 (m, 2H), 2.86 (t, J=5.0 Hz, 4H), 2.72-2.58 (m, 3H), 2.58-2.44 (m, 6H), 2.01 (dq, J=12.5, 2.7 Hz, 1H), 1.76 (d, J=12.9 Hz, 1H), 1.66-1.51 (m, 3H), 1.31 (s, 1H), 1.22-0.98 (m, 2H). ¹³C NMR (75 MHz, MeOD) δ 174.5, 142.3, 127.9, 125.3, 59.9, 57.2, 53.3, 45.0, 44.7, 38.8, 36.6, 35.5, 35.2, 32.2, 32.0.

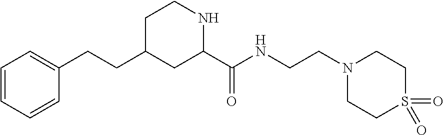 [SMILES: O=C(NCCN1CCS(=O)(=O)CC1)C1CC(CCc2ccccc2)CCN1]

N-(2-(1,1-dioxidothiomorpholino)ethyl)-4-phenethylpiperidine-2-carboxamide (AB0132). The synthesis of compound AB0132 was conducted by following a procedure similar to that of compound AB0146. Yield 14 mg as a colorless gel, 100%. ¹H NMR (300 MHz, MeOD) δ 7.33-7.09 (m, 4H), 3.23-2.99 (m, 8H), 2.73-2.52 (m, 4H), 2.00 (dq, J=12.6, 2.9 Hz, 1H), 1.82-1.70 (m, 1H), 1.66-1.43 (m, 3H), 1.36-1.28 (m, 1H), 1.22-0.98 (m, 2H). ¹³C NMR (75 MHz, MeOD) δ 174.6, 142.3, 127.9, 125.3, 59.9, 54.7, 50.7, 50.4, 45.0, 38.8, 36.7, 36.2, 35.2, 32.2, 32.0.

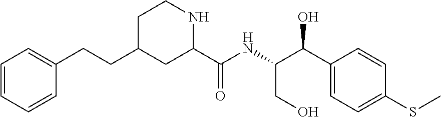 [SMILES: CSc1ccc([C@H](O)[C@H](CO)NC(=O)C2CC(CCc3ccccc3)CCN2)cc1]

N-((1S,2S)-1,3-dihydroxy-1-(4-(methylthio)phenyl)propan-2-yl)-4-phenethylpiperidine-2-carboxamide (AB0135). The synthesis of compound AB0135 was conducted by following a procedure similar to that of compound AB0146. Yield 15 mg as a colorless gel, 62%. ¹H NMR (300 MHz, MeOD) δ 7.50-6.80 (m, 12H), 4.98 (t, J=4.2 Hz, 1H), 4.24-4.03 (m, 1H), 3.84 (dd, J=11.2, 5.1 Hz, OH), 3.78-3.65 (m, 1H), 3.55 (ddd, J=10.9, 5.8, 3.1 Hz, 1H), 3.07 (dddd, J=20.9, 14.4, 11.7, 2.8 Hz, 3H), 2.72-2.45 (m, 4H), 2.45-2.35 (m, 4H), 1.89-1.60 (m, 3H), 1.62-0.53 (m, 13H). ¹³C NMR (75 MHz, MeOD) δ 175.2, 174.5, 142.3, 139.4, 139.2, 137.4, 137.3, 127.9, 127.9, 127.4, 127.2, 126.4, 125.9, 125.7, 125.7, 125.3, 72.8, 70.7, 70.6, 61.2, 60.9, 60.2, 59.6, 56.4, 56.2, 55.4, 44.9, 44.8, 44.7, 38.9, 38.8, 38.7, 36.6, 36.5, 36.3, 35.2, 35.1, 35.0, 32.2, 32.1, 32.0, 14.4, 14.2.

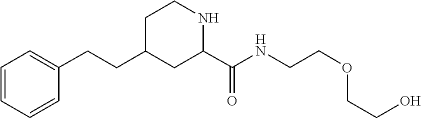 [SMILES: O=C(NCCOCCO)C1CC(CCc2ccccc2)CCN1]

N-(2-(2-hydroxyethoxy)ethyl)-4-phenethylpiperidine-2-carboxamide (AB0136). The synthesis of compound AB0136 was conducted by following a procedure similar to that of compound AB0146. Yield 10 mg as a colorless gel, 52%. ¹H NMR (300 MHz, MeOD) δ 7.33-7.09 (m, 5H), 3.69 (dd, J=5.5, 3.9 Hz, 2H), 3.64-3.51 (m, 4H), 3.41 (t, J=5.3 Hz, 2H), 3.31-3.10 (m, 2H), 2.73-2.57 (m, 3H), 2.04 (dq, J=12.7, 2.7 Hz, 1H), 1.67-1.49 (m, 3H), 1.36-1.28 (m, 1H), 1.26-1.03 (m, 2H). ¹³C NMR (75 MHz, MeOD) δ 173.8, 142.2, 127.9, 125.3, 72.0, 69.0, 60.7, 59.6, 44.7, 38.8, 38.6, 36.1, 35.0, 32.2, 31.5.

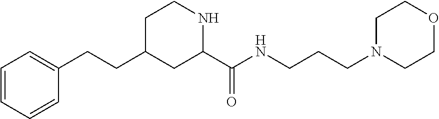 [SMILES: O=C(NCCCN1CCOCC1)C1CC(CCc2ccccc2)CCN1]

N-(3-morpholinopropyl)-4-phenethylpiperidine-2-carboxamide (AB0145). The synthesis of compound AB0145 was conducted by following a procedure similar to that of compound AB0146. Yield 3.2 mg as a colorless gel, 45%. ¹H NMR (300 MHz, MeOD) δ 7.33-7.09 (m, 5H), 3.77-3.67 (m, 4H), 3.26 (t, J=6.8 Hz, 5H), 3.20-3.02 (m, 2H), 2.72-2.53 (m, 3H), 2.53-2.35 (m, 5H), 2.04-1.92 (m, 1H), 1.81-1.66 (m, 3H), 1.66-1.47 (m, 3H), 1.32 (d, J=4.2 Hz, 2H), 1.22-0.97 (m, 2H). ¹³C NMR (75 MHz, MeOD) δ 174.4, 142.3, 127.9, 125.3 66.2 59.9, 56.2 53.3, 44.9, 38.8, 37.3, 36.6, 35.2, 32.2, 32.0, 25.4.

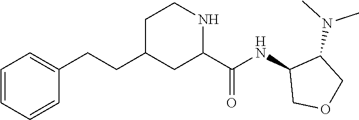 [SMILES: CN(C)[C@@H]1COC[C@H]1NC(=O)C1CC(CCc2ccccc2)CCN1]

N-((3S,4S)-4-(dimethylamino)tetrahydrofuran-3 yl)-4-phenethylpiperidine-2-carboxamide (AB0281). The synthesis of compound AB0281 was conducted by following a procedure similar to that of compound AB0146. Yield 24.1 mg, 51%. ¹H NMR (300 MHz, MeOD) δ 7.23 (tt, J=13.8, 7.3 Hz, 5H), 4.42 (q, J=4.9 Hz, 1H), 4.05 (t, J=8.1 Hz, 2H), 3.69 (dd, J=9.5, 6.0 Hz, 1H), 3.58 (dd, J=9.4, 4.8 Hz, 1H), 3.35 (s, 1H), 3.20 (q, J=11.4, 10.2 Hz, 2H), 2.88 (p, J=6.2, 5.2 Hz, 1H), 2.75-2.55 (m, 3H), 2.31 (s, 5H), 1.97 (d, J=12.8 Hz, 1H), 1.79 (d, J=13.0 Hz, 1H), 1.58 (h, J=9.1, 7.9 Hz, 3H), 1.12 (h, J=12.0 Hz, 2H). ¹³C NMR (75 MHz, MeOD) δ 173.6, 142.3, 128.0, 125.4, 72.9, 72.3, 70.5, 59.6, 52.2, 44.9, 42.2, 38.7, 36.5, 35.2, 32.2, 32.0. HRMS (ESI) calcd for C₂₀H₃₁N₃O₂, 345.4870 [M+H]⁺; found,

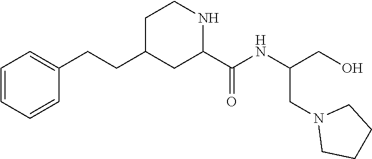 [SMILES: O=C(NC(CO)CN1CCCC1)C1CC(CCc2ccccc2)CCN1]

N-(1-hydroxy-3-(pyrrolidin-1-yl)propan-2-yl)-4-phenethylpiperidine-2-carboxamide (AB0282). The synthesis of compound AB0282 was conducted by following a procedure similar to that of compound AB0146. Yield 33.9 mg, 69%. ¹H NMR (300 MHz, MeOD) δ 7.36-7.11 (m, 5H), 4.14 (tq, J=7.2, 3.7, 2.2 Hz, 1H), 3.60 (d, J=5.3 Hz, 2H), 3.41-3.12 (m, 3H), 2.87-2.57 (m, 9H), 2.10 (dtd, J=12.6, 5.2, 2.6 Hz, 1H), 1.95-1.71 (m, 5H), 1.73-1.49 (m, 4H), 1.21 (s, 2H). ¹³C NMR (75 MHz, MeOD) δ 173.7, 142.2, 128.0, 128.0, 127.9, 125.4, 62.6, 62.5, 59.8, 59.7, 56.6, 54.1, 54.0, 49.7, 49.6, 44.8, 44.7, 38.6, 38.6, 35.9, 35.6, 35.0, 34.9, 32.2 31.4, 31.2, 22.9, 22.8.

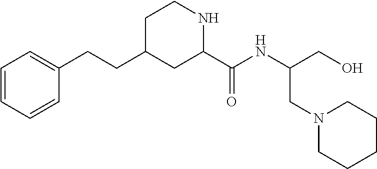 [SMILES: O=C(NC(CO)CN1CCCCC1)C1CC(CCc2ccccc2)CCN1]

N-(1-hydroxy-3-(piperidin-1-yl)propan-2-yl)-4 phenethylpiperidine-2-carboxamide (AB0283). The synthesis of compound AB0283 was conducted by following a procedure similar to that of compound AB0146. Yield 19.4 mg, 38%. ¹H NMR (300 MHz, MeOD) δ 7.37-7.05 (m, 5H), 4.36-3.91 (m, 2H), 3.79-3.37 (m, 5H), 3.08-2.54 (m, 6H), 2.09-1.14 (m, 17H). ¹³C NMR (75 MHz, MeOD) δ 174.3, 142.1, 128.0, 127.9, 125.4, 80.4, 63.5, 63.0, 62.1, 59.7, 57.0, 54.3, 36.9, 32.8, 27.3, 24.4.

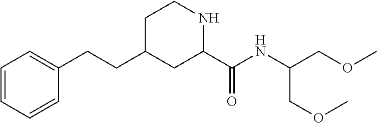 [SMILES: COCC(COC)NC(=O)C1CC(CCc2ccccc2)CCN1]

N-(1,3-dimethoxypropan-2-yl)-4-phenethylpiperidine-2-carboxamide (AB0284). The synthesis of compound AB0284 was conducted by following a procedure similar to that of compound AB0146. Yield 12 mg, 26%. ¹H NMR (300 MHz, MeOD) δ 7.40-7.03 (m, 5H), 4.19 (p, J=5.6 Hz, 1H), 3.64-3.55 (m, 1H), 3.47 (dd, J=5.5, 2.1 Hz, 4H), 3.35 (d, J=3.4 Hz, 7H), 2.87 (td, J=13.0, 3.1 Hz, 1H), 2.74-2.61 (m, 2H), 2.23-2.12 (m, 1H), 2.00-1.86 (m, 1H), 1.77-1.52 (m, 3H), 1.40-1.20 (m, 2H). ¹³C NMR (75 MHz, MeOD) δ 170.5, 141.9, 128.0, 127.9, 125.5 71.0, 58.6, 57.9, 57.9, 48.8, 47.6, 47.3, 47.0, 46.8, 43.8, 38.0, 34.7, 34.1, 32.2, 29.2.

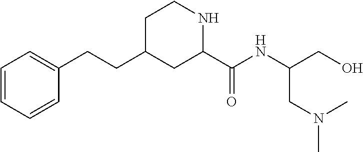 [SMILES: CN(C)CC(CO)NC(=O)C1CC(CCc2ccccc2)CCN1]

N-(1-(dimethylamino)-3-hydroxypropan-2-yl)-4-phenethylpiperidine-2-carboxamide (AB0285). The synthesis of compound AB0285 was conducted by following a procedure similar to that of compound AB0146. Yield 12.5 mg, 27%. ¹H NMR (300 MHz, MeOD) δ 7.34-7.06 (m, 5H), 4.57-4.40 (m, 1H), 4.07 (s, 1H), 3.82-3.40 (m, 7H), 3.39-3.17 (m, 9H), 2.66 (t, J=7.6 Hz, 3H), 2.11-1.93 (m, 2H), 1.87 (dq, J=9.3, 5.1, 4.2 Hz, 2H), 1.73-1.55 (m, 5H), 1.53-1.25 (m, 16H). ¹³C NMR (75 MHz, MeOD) δ 174.1, 142.1, 128.0, 127.9, 125.4, 80.2, 71.3, 62.6, 58.4, 57.0, 56.2, 48.4, 48.2, 47.9, 47.6, 47.5, 47.3, 47.3, 47.1, 47.0, 46.7, 36.9, 32.8, 31.2, 29.3, 27.2.

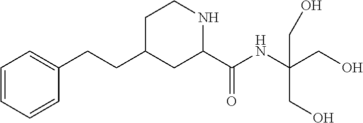 [SMILES: O=C(NC(CO)(CO)CO)C1CC(CCc2ccccc2)CCN1]

N-(1,3-dihydroxy-2-(hydroxymethyl)propan-2-yl)-4 phenethylpiperidine-2-carboxamide (AB0288). The synthesis of compound AB0288 was conducted by following a procedure similar to that of compound AB0146. Yield 40 mg, 88%. ¹H NMR (300 MHz, MeOD) δ 7.37-7.03 (m, 5H), 3.76 (s, 1H), 3.63 (s, 1H), 3.48-3.35 (m, 1H), 2.92 (td, J=13.1, 3.2 Hz, 1H), 2.68 (q, J=7.2 Hz, 2H), 2.44 (d, J=13.8 Hz, 1H), 1.94 (d, J=13.9 Hz, 1H), 1.63 (q, J=6.9, 6.4 Hz, 4H), 1.51-1.18 (m, 4H), 0.91 (d, J=7.1 Hz, 1H). ¹³C NMR (75 MHz, MeOD) δ 172.7, 141.9, 128.0, 128.0, 125.5, 125.4, 125.4, 61.3, 59.5, 59.3, 58.1, 43.2, 37.8, 34.0, 33.1, 32.3, 32.2, 28.2.

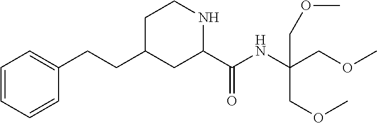 [SMILES: COCC(COC)(COC)NC(=O)C1CC(CCc2ccccc2)CCN1]

N-(1,3-dimethoxy-2-(methoxymethyl)propan-2-yl)-4-phenethylpiperidine-2-carboxamide (AB0289). The synthesis of compound AB0289 was conducted by following a procedure similar to that of compound AB0146. Yield 20.6 mg, 40%. ¹H NMR (300 MHz, MeOD) δ 7.45-6.95 (m, 5H), 3.69 (d, J=12.4 Hz, 5H), 3.36 (d, J=1.9 Hz, 11H), 2.70 (q, J=8.1 Hz, 3H), 2.08 (dt, J=13.0, 2.7 Hz, 1H), 1.82 (dd, J=13.2, 3.4 Hz, 1H), 1.72-1.37 (m, 3H), 1.43-0.94 (m, 2H). ¹³C NMR (75 MHz, MeOD) δ 173.5, 142.2, 128.1, 128.0, 128.0, 125.4, 70.7, 70.6, 59.9, 59.6, 58.2, 58.2, 44.6, 38.5, 36.2, 34.9, 32.3, 31.1.

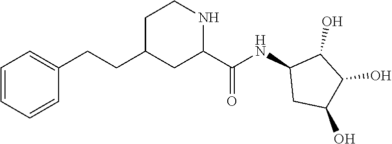 [SMILES: O=C(N[C@@H]1C[C@H](O)[C@@H](O)[C@H]1O)C1CC(CCc2ccccc2)CCN1]

4-phenethyl-N-((1R,2S,3R,4S)-2,3,4-trihydroxycyclopentyl)piperidine-2-carboxamide (AB0292). The synthesis of compound AB0292 was conducted by following a procedure similar to that of compound AB0146. Yield 20 mg, 42%. ¹H NMR (300 MHz, Chloroform-d) δ 7.21 (dt, J=18.0, 8.7 Hz, 5H), 4.17-3.49 (m, 2H), 3.53-3.22 (m, 3H), 2.92 (t, J=12.7 Hz, 1H), 2.68 (t, J=7.5 Hz, 3H), 2.44 (d, J=14.1 Hz, 1H), 1.93 (d, J=14.2 Hz, 2H), 1.64 (s, 4H), 1.33 (dq, J=24.4, 12.4, 11.7 Hz 3H). ¹³C NMR (75 MHz, MeOD) δ 172.7, 141.9, 128.1, 128.0, 125.5, 77.1, 76.1, 74.3, 59.5, 43.2, 37.8, 34.0, 33.1, 32.2, 32.1, 28.2.

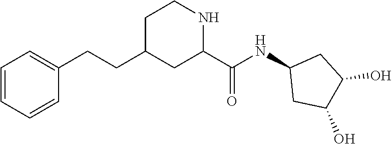 [SMILES: O=C(N[C@@H]1C[C@@H](O)[C@@H](O)C1)C1CC(CCc2ccccc2)CCN1]

N-((1s,3R,4S)-3,4-dihydroxycyclopentyl)-4-phenethylpiperidine-2-carboxamide (AB0293). The synthesis of compound AB0293 was conducted by following a procedure similar to that of compound AB0146. Yield 19.1 mg, 42%. ¹H NMR (300 MHz, Chloroform-d) δ 7.37-7.03 (m, 5H), 6.13 (d, J=7.4 Hz, 1H), 4.49 (q, J=6.5 Hz, 1H), 4.34-4.13 (m, 3H), 3.81-3.57 (m, 1H), 3.19 (ddd, J=14.0, 9.8, 4.8 Hz, 1H), 2.66 (ddd, J=14.7, 10.9, 6.7 Hz, 3H), 2.34-2.09 (m, 3H), 2.13-1.53 (m, 4H), 1.43-1.16 (m, 3H), 1.04-0.78 (m, 3H). ¹³C NMR (75 MHz MeOD) δ 172.1, 141.9, 128.0, 127.9, 125.5, 72.5, 58.5, 48.5, 48.2, 47.9 47.6, 47.3 47.0, 46.8, 43.8, 37.9, 37.7, 34.5, 34.0, 32.2, 27.2.

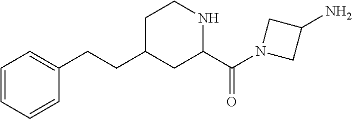 [SMILES: NC1CN(C(=O)C2CC(CCc3ccccc3)CCN2)C1]

(3-aminoazetidin-1-yl)(4-phenethylpiperidin-2-yl)methanone (AB0294). The synthesis of compound AB0294 was conducted by following a procedure similar to that of compound AB0146. Yield 6.3 mg, 16%. ¹H NMR (300 MHz, MeOD) δ 7.48-6.97 (m, 5H), 3.73 (hept, J=6.6 Hz, 1H), 3.42 (ddd, J=16.6, 9.7, 3.5 Hz, 1H), 3.23 (q, J=7.4 Hz, 2H), 3.02-2.85 (m, 1H), 2.69 (t, J=7.4 Hz, 2H), 2.44 (d, J=13.9 Hz, 1H), 1.94 (d, J=13.4 Hz, 1H), 1.64 (dd, J=7.9, 4.8 Hz, 2H), 1.53-1.18 (m, 8H). ¹³C NMR (75 MHz, MeOD) 13C NMR (75 MHz, MeOD) δ 161.9, 141.9, 128.0, 127.9, 125.5, 59.5, 54.4, 43.3, 42.4, 37.7, 34.0, 33.1, 32.2, 28.1.

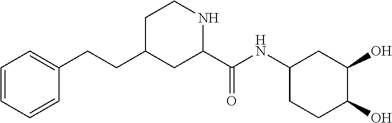 [SMILES: O=C(NC1CC[C@H](O)[C@H](O)C1)C1CC(CCc2ccccc2)CCN1]

N-((3R,4S)-3,4-dihydroxycyclohexyl)-4-phenethylpiperidine-2-carboxamide (AB0295). The synthesis of compound AB0295 was conducted by following a procedure similar to that of compound AB0146. Yield 8.2 mg, 17%. ¹H NMR (300 MHz, MeOD) δ 7.23 (dq, J=16.5, 8.4, 8.0 Hz, 5H), 4.15-3.89 (m, 1H), 3.93-3.53 (m, 2H), 3.52-3.36 (m, 1H), 3.13-2.86 (m, 1H), 2.68 (t, J=8.0 Hz, 2H), 2.43 (d, J=13.9 Hz, 1H), 2.22 (d, J=13.9 Hz, 1H), 2.11-1.28 (m, 12H). ¹³C NMR (75 MHz, MeOD) 13C NMR (75 MHz, MeOD) δ 168.3, 168.0, 141.8, 128.1, 128.0, 125.5, 70.4, 69.9, 68.8, 68.1, 59.5, 58.0, 54.4, 46.6, 43.6, 43.5, 43.3, 42.4, 37.8, 37.6, 36.0, 34.0, 33.8, 33.7, 33.5, 33.1, 32.2, 28.9, 28.1, 27.9, 27.7, 26.5, 25.2.

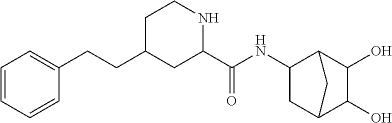 [SMILES: O=C(NC1CC2CC1C(O)C2O)C1CC(CCc2ccccc2)CCN1]

N-(5,6-dihydroxybicyclo[2.2.1]heptan-2-yl)-4-phenethylpiperidine-2-carboxamide (AB0296). The synthesis of compound AB0296 was conducted by following a procedure similar to that of compound AB0146. Yield 6.3 mg, 13%. ¹H NMR (300 MHz, MeOD) δ 7.23 (dq, J=16.4, 8.1, 7.4 Hz, 5H), 3.99 (ddd, J=16.5, 11.2, 5.4 Hz, 1H), 3.87-3.52 (m, 3H), 3.50-3.36 (m, 1H), 3.23 (q, J=7.4 Hz, 1H), 3.02 (td, J=13.2, 3.4 Hz, 1H), 2.69 (td, J=8.4, 7.9, 4.0 Hz, 2H), 2.40-1.58 (m, 9H), 1.52-1.23 (m, 6H), 1.02-0.77 (m, 1H). ¹³C NMR (75 MHz, MeOD) 13C NMR (75 MHz, MeOD) δ 168.9, 141.8, 128.1, 128.0, 125.6, 73.8, 73.0, 71.4, 68.6, 68.6, 58.0, 57.9, 54.4, 49.1, 49.0, 48.9, 47.0, 43.3, 42.4, 37.7, 37.6, 33.9, 33.7, 33.5, 32.1, 31.1, 30.7, 30.6, 27.8.

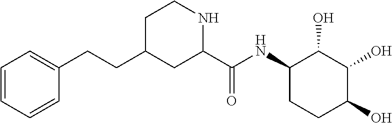 [SMILES: O=C(N[C@@H]1CC[C@H](O)[C@@H](O)[C@H]1O)C1CC(CCc2ccccc2)CCN1]

4-phenethyl-N-((1R,2S,3R,4S)-2,3,4-trihydroxycyclohexyl)piperidine-2-carboxamide (AB0297). The synthesis of compound AB0297 was conducted by following a procedure similar to that of compound AB0146. Yield 5.9 mg, 16%. ¹H NMR (300 MHz, MeOD) ¹H NMR δ 7.23 (dq, J=17.2, 8.9, 8.2 Hz, 5H), 4.13-3.61 (m, 3H), 3.53-3.37 (m, 1H), 3.12-2.82 (m, 1H), 2.69 (t, J=7.6 Hz, 2H), 2.56-2.20 (m, 1H), 2.13-1.78 (m, 2H), 1.83-1.53 (m, 4H), 1.51-1.12 (m, 5H). ¹³C NMR (75 MHz, MeOD) δ 168.7, 141.8, 128.1, 128.0, 125.5, 73.1, 70.7, 69.6, 69.5, 59.5, 58.1, 54.4, 49.6, 48.5, 48.2, 47.9, 47.6, 47.3, 47.0, 46.8, 43.3, 43.2, 42.4, 37.7, 37.6, 33.9, 33.6, 33.1, 32.2, 28.1, 27.8, 25.8, 24.6, 11.7.

100 mg of racemic CTW0404 and CTW0419 were separated by chiral prep-SFC. As for CTW0419, a further separation was performed to increase the e.e.% of the enantiomers. After separation, 41.1 mg of faster eluting enantiomer and 40.9 mg of slower eluting enantiomer of CTW0404 were obtained respectively. As for CTW0419, there're 20.0 mg and 17.7 mg of two enantiomers were obtained respectively.

### Separation Results

| | | | E.E. by | Purity |
|---|---|---|---|---|
| | | | chiral | by |
| Compound ID and Name, | | Weight | HPLC | LCMS |
| with absolute configuration | Description | (mg) | (%) | (%) |
| CTW0404-P1 | Enantiomer 1, | 41.1 | 100% | 95.67% |
| (a cis-CTW0404 isomer) | faster eluting | | | |
| (2S,4R)-N-(2-morpholinoethyl)-4- | isomer by chiral | | | |
| phenethylpiperidine-2-carboxamide | HPLC on AS | | | |
| | column | | | |
| CTW0404-P2 | Enantiomer 2, | 40.9 | 100% | 94.29% |
| (a cis-CTW0404 isomer) | slower eluting | | | |
| (2R,4S)-N-(2-morpholinoethyl)-4- | isomer by chiral | | | |
| phenethylpiperidine-2-carboxamide | HPLC on AS | | | |
| | column | | | |
| CTW0419-P1 | Enantiomer 1, | 20.0 | 96.22% | 100% |
| (a cis-CTW0419 isomer) | faster eluting | | | |
| (2S,4R)-N-(1,3-dihydroxypropan-2-yl)-4- | isomer by chiral | | | |
| phenethylpiperidine-2-carboxamide | HPLC on AD | | | |
| | column | | | |
| CTW0419-P2 | Enantiomer 2, | 17.7 | 99.12% | 100% |
| (a cis-CTW0419 isomer) | slower eluting | | | |
| (2R,4S)-N-(1,3-dihydroxypropan-2-yl)-4- | isomer by chiral | | | |
| phenethylpiperidine-2-carboxamide | HPLC on AD | | | |
| | column | | | |

### CTW0404 Separation Method

### Analytical Separation Method

Instrument: Waters Acquity Arc HPLC system;
Column: Chiralpak AS-3, 100×4.6 mm i.d., 3 μm;
Mobile Phase: A for n-hexane and B for ethanol (0.1% IPAm, v/v);
Gradient: A:B=85:15 isocratic elution mode;
Flow rate: 1.0 ml/min;
Column temperature: 30° C.;
IPAm: isopropylamine

### Preparative Separation Method

Instrument: Waters SFC150AP preparative SFC
Column: ChiralPak AS, 250×30 mm I.D. 10 μm
Mobile phase: A for CO2 and B for ethanol (0.1% NH₃H₂O, v/v)
Gradient: B %=25% isocratic elution mode;
Flow rate: 60 g/min
Column temperature: 35° C.;
System back pressure: 100 bar
Sample preparation: Racemic material was dissolved in ethanol to 5 mg/mL and filtrated through membrane with pore sized 0.45 um.
Injection: 4.0 mL per injection.
Work up: After separation, the fractions were dried off via rotary evaporator at bath temperature 35° C. to get the two enantiomers.

### CTW0419 Separation Method

Analytical Separation Method
Instrument: Waters Acquity Arc HPLC system;
Column: Chiralpak AD-3, 100×4.6 mm i.d., 3 μm;
Mobile Phase: A for n-hexane and B for ethanol (0.1% IPAm, v/v);
Gradient: A:B=80:20 isocratic elution mode;
Flow rate: 1.0 ml/min;
Column temperature: 30° C.;

### Preparative Separation Method

Instrument: Waters SFC150AP preparative SFC
Column: ChiralPak AD, 250×30 mm I.D. 10 μm
Mobile phase: A for CO2 and B for methanol (0.1% NH₃H₂O, v/v)
Gradient: B %=30% isocratic elution mode;
Flow rate: 70 g/min
Column temperature: 35° C.;
System back pressure: 100 bar
Sample preparation: Racemic material was dissolved in methanol to 3 mg/mL and
filtrated through membrane with pore sized 0.45 um.
Injection: 3.0 mL per injection.
Work up: After separation, the fractions were dried off via rotary evaporator at bath temperature 35° C. to get the two enantiomers.

### Quality Control

### E.E. Test Via Chiral HPLC

After concentration, e.e. value for the isomers was tested by the method described above.

### Purity Test Via RP-LCMS

After concentration, RPLC-MS was used to test the purity of the enantiomers.

Structure Confirmation via NMR

After concentration, NMR was used to characterize the structures.

### CTW0404-P1

¹H NMR (300 MHz, CDCl₃) δ 7.33-7.25 (m, 2H), 7.19 (td, J=6.1, 1.6 Hz, 3H), 6.94 (d, J=4.5 Hz, 1H), 3.78-3.68 (m, 4H), 3.38 (q, J=6.0 Hz, 2H), 3.23-3.11 (m, 2H), 2.73-2.61 (m, 3H), 2.55-2.43 (m, 6H), 2.19 (dq, J=12.7, 2.9 Hz, 1H), 1.75 (dq, J=10.9, 2.9 Hz, 3H), 1.65-1.53 (m, 2H), 1.19-0.92 (m, 2H).

### CTW0404-P2

¹H NMR (300 MHz, CDCl₃) δ 7.35-7.25 (m, 2H), 7.19 (td, J=6.2, 1.6 Hz, 3H), 6.96 (s, 1H), 3.81-3.67 (m, 4H), 3.39 (q, J=6.0 Hz, 2H), 3.18 (tt, J=8.7, 2.3 Hz, 2H), 2.76-2.60 (m, 3H), 2.56-2.42 (m, 6H), 2.19 (dq, J=12.8, 2.9 Hz, 1H), 1.93-1.69 (m, 3H), 1.65-1.42 (m, 2H), 1.24-0.95 (m, 2H).

### CTW0419-P1

¹H NMR (300 MHz, CDCl₃) δ 7.30-7.22 (m, 2H), 7.15 (d, J=7.6 Hz, 3H), 3.86 (q, J=4.8 Hz, 1H), 3.77-3.60 (m, 4H), 3.13 (ddd, J=10.5, 7.6, 3.2 Hz, 2H), 2.60 (q, J=8.6 Hz, 3H), 2.05 (d, J=12.8 Hz, 1H), 1.73 (d, J=13.2 Hz, 1H), 1.63-1.39 (m, 3H), 1.27-0.97 (m, 3H).

### CTW0419-P2

¹H NMR (300 MHz, CDCl₃) δ 7.33-7.21 (m, 2H), 7.20-7.10 (m, 3H), 3.92-3.79 (m, 1H), 3.74-3.56 (m, 4H), 3.18-3.08 (m, 2H), 2.70-2.49 (m, 3H), 2.04 (d, J=12.9 Hz, 1H), 1.72 (d, J=13.3 Hz, 1H), 1.60-1.38 (m, 3H), 1.05 (q, J=12.2 Hz, 3H).

Absolute Chiral Configuration Test via X-ray Analysis confirm the absolute configuration of each chiral center.

General Method for In Vitro Pharmacological Assessment of Compounds of the Invention. The Chinese hamster ovary (CHO) cell lines stably transfected with human unedited (INI) h5-HT_{2C}R (h5-HT_{2C}R—CHO cells) or the human h5-HT_{2A}R (h5-HT_{2A}R—CHO cells) were a generous gift of K. Berg and W. Clarke (University of Texas Health Science Center, San Antonio, TX) (Berg et al., Molecular Pharmacology, 46:477-484, 1994; Ding et al., ACS Chemical Neuroscience, 3:538-545, 2012). Cells were grown at 37° C., 5% CO₂ and 85% relative humidity in GlutaMax α-MEM (Invitrogen, Carlsbad, CA), 5% fetal bovine serum (Atlanta Biologicals, Atlanta, GA), 100 μg/mL hygromycin (Mediatech, Manassas Va.) and were passaged when they reached 80% confluence. Changes in Caᵢ²⁺ levels were determined using the calcium sensitive dye Calcium 4 or Calcium 6 (FLIPR No-wash kit, Molecular Devices, Sunnyvale, CA). Specifically, cells (150 μL; passages 6-16) were plated in serum-replete medium at a density of 14,000-16,000 (FlexStation 3; Molecular Devices) or 30,000 cells/well (FLIPR^{TETRA}; Molecular Devices) in black-wall 96-well culture plates with optically clear flat bottoms. After −24 hours, the medium was replaced with serum-free (SF) GlutaMax-MEM medium supplemented with 20 nM to 100 μM putrescine (Sigma-Aldrich, St. Louis, MO), 20 nM to 100 μM progesterone (Sigma-Aldrich), and 1:100 ITS (1000 mg/L human recombinant insulin, 550 mg/L human recombinant transferrin, 0.67 mg/L selenious acid; Corning Inc., Corning, NY) (SF+ medium). After an incubation for another 3 h, SF+ medium was replaced with 40 μL of Hank's balanced saline solution (HBSS; without CaCl₂ or MgCl₂, pH 7.4) plus 40 μL of Calcium dye solution supplemented with 2.5 mM of water-soluble probenecid (Sigma-Aldrich), and then the plate was incubated with dye solution in the dark for 1 h at 37° C., 15 min at room temperature. The compound was diluted at 5× concentration in 1×HBSS. The delivery of compound (20 μL/well) was 15 min prior to the addition of 5-HT (25 μL/well), and a baseline was established for each well before the addition of the compound and 5-HT. The fluorescence readings were then adopted to evaluate the allosteric modulation of 5-HT-induced Caᵢ²⁺ release. FlexStation 3 or FLIPR^{TETRA} was used to measure fluorescence. For FlexStation 3, a 17 s baseline was established before the compound was added, and fluorescence was recorded every 1.7 s thereafter for 240 s. The maximum peak height of each well was determined by SoftMax software (Pro 5.4.5). For FLIPR^{TETRA}, a 10 s baseline was established before adding the compound, and then record the fluorescence every 1 s for 120 s after the compound or 360 s after 5-HT. The maximum peak height of each well was determined by ScreenWorks 4.0 software. After the final reading, the cells were fixed in 2% paraformaldehyde (Sigma) overnight. A 4-parameter nonlinear regression analysis was used to determine the 5-HT-induced Caᵢ²⁺ maximum release (Eₘₐₓ) in the presence of the test compound, and calculated from 4-6 biological replicates, each biological replicate performed in technical triplicates. The Eₘₐₓ of the test compound plus 5-HT was normalized to the Eₘₐₓ of 5-HT alone. Subsequently, Welch's unpaired t-test was used for comparison of the Eₘₐₓ means. All statistical analyses were performed with an experimental error rate of α=0.05.

### REFERENCES

1. Abbott, F. V., Y. Hong, and P. Blier. 1997. ‘Persisting sensitization of the behavioural response to formalin-induced injury in the rat through activation of serotonin2A receptors’, Neuroscience, 77: 575-84.
2. Barnes, N. M., G. P. Ahern, C. Becamel, J. Bockaert, M. Camilleri, S. Chaumont-Dubel, S. Claeysen, K. A. Cunningham, K. C. Fone, M. Gershon, G. Di Giovanni, N. M. Goodfellow, A. L. Halberstadt, R. M. Hartley, G. Hassaine, K. Herrick-Davis, R. Hovius, E. Lacivita, E. K. Lambe, M. Leopoldo, F. O. Levy, S. C. R. Lummis, P. Marin, L. Maroteaux, A. C. McCreary, D. L. Nelson, J. F. Neumaier, A. Newman-Tancredi, H. Nury, A. Roberts, B. L. Roth, A. Roumier, G. J. Sanger, M. Teitler, T. Sharp, C. M. Villalon, H. Vogel, S. W. Watts, and D. Hoyer. 2021. ‘International Union of Basic and Clinical Pharmacology. CX. Classification of Receptors for 5-hydroxytryptamine; Pharmacology and Function’, Pharmacological Reviews, 73: 310-520.
3. Berg, K. A., W. P. Clarke, C. Sailstad, A. Saltzman, and S. Maayani. 1994. ‘Signal transduction differences between 5-hydroxytryptamine type 2A and type 2C receptor systems’, Molecular Pharmacology, 46: 477-84.
4. Christopoulos, A. 2014. ‘Advances in GPCR allostery: From function to structure’, Molecular Pharmacology.
5. Conn, P. J., A. Christopoulos, and C. W. Lindsley. 2009. ‘Allosteric modulators of GPCRs: a novel approach for the treatment of CNS disorders’, Nature Reviews: Drug Discovery, 8: 41-54.
6. Couch, Y., C. J. Martin, C. Howarth, J. Raley, A. A. Khrapitchev, M. Stratford, T. Sharp, N. R. Sibson, and D. C. Anthony. 2013. ‘Systemic inflammation alters central 5-HT function as determined by pharmacological MRI’, Neuroimage, 75: 177-86.
7. Couch, Y., Q. Xie, L. Lundberg, T. Sharp, and D. C. Anthony. 2015. ‘A Model of Post-Infection Fatigue Is Associated with Increased TNF and 5-HT2A Receptor Expression in Mice’, PloS One, 10: e0130643.
8. Cunningham, K. A., L. L. Howell, and N. C. Anastasio. 2020. ‘Serotonin neurobiology in cocaine use disorder.’ in C. P. Muller and K. A. Cunningham (eds.), Handbook of the Behavioral Neurobiology of Serotonin (Academic Press: United Kingdom).
9. Ding, C., N. M. Bremer, T. D. Smith, P. K. Seitz, N. C. Anastasio, K. A. Cunningham, and J. Zhou. 2012. ‘Exploration of synthetic approaches and pharmacological evaluation of PNU-69176E and its stereoisomer as 5-HT_{2C} receptor allosteric modulators’, ACS Chemical Neuroscience, 3: 538-45.
10. Elphick, G. F., W. Querbes, J. A. Jordan, G. V. Gee, S. Eash, K. Manley, A. Dugan, M. Stanifer, A. Bhatnagar, W. K. Kroeze, B. L. Roth, and W. J. Atwood. 2004. ‘The human polyomavirus, JCV, uses serotonin receptors to infect cells’, Science, 306: 1380-3.
11. Flanagan, T. W., M. N. Sebastian, D. M. Battaglia, T. P. Foster, S. A. Cormier, and C. D. Nichols. 2019. ‘5-HT2 receptor activation alleviates airway inflammation and structural remodeling in a chronic mouse asthma model’, Life Sciences, 236: 116790.
12. Howell, L. L., and K. A. Cunningham. 2015. ‘Serotonin 5-HT2 receptor interactions with dopamine function: implications for therapeutics in cocaine use disorder’, Pharmacological Reviews, 67: 176-97.
13. May, L. T., K. Leach, P. M. Sexton, and A. Christopoulos. 2007. ‘Allosteric modulation of G protein-coupled receptors’, Annual Review of Pharmacology and Toxicology, 47: 1-51.
14. Nichols, D. E. 2004. ‘Hallucinogens’, Pharmacology and Therapeutics, 101: 131-81.
15. Orejarena, M. J., L. Lanfumey, R. Maldonado, and P. Robledo. 2011. ‘Involvement of 5-HT2A receptors in MDMA reinforcement and cue-induced reinstatement of MDMA-seeking behaviour’, International Journal of Neuropsychopharmacology, 14: 927-40.
16. Wild, C. T., J. M. Miszkiel, E. A. Wold, C. A. Soto, C. Ding, R. M. Hartley, M. A. White, N. C. Anastasio, K. A. Cunningham, and J. Zhou. 2019. ‘Design, synthesis, and characterization of 4-undecylpiperidine-2-carboxamides as positive allosteric modulators of the serotonin (5-HT) 5-HT2C receptor’, Journal of Medicinal Chemistry, 62: 288-305.
17. Wold, E. A., E. J. Garcia, C. T. Wild, J. M. Miszkiel, C. A. Soto, J. Chen, K. Pazdrak, R. G. Fox, N. C. Anastasio, K. A. Cunningham, and J. Zhou. 2020. ‘Discovery of 4-phenylpiperidine-2-carboxamide analogues as serotonin 5-HT2C receptor-positive allosteric modulators with enhanced drug-like properties’, Journal of Medicinal Chemistry, 63: 7529-44.

### Published Manuscripts, Book Chapters, and Publicly Available Pre-Prints

1. Chen, H. J., Zhou, X., Gao, Y., Chen, H. Y, Zhou. J. Chapter 8: Fragment-based drug design: Strategic advances and lessons learned. In Comprehensive Medicinal Chemistry III (ISBN: 978-0-128-03200-8), Vol 2: Drug Discovery Technologies, 1st ed.; Samuel Chackalamannil, David P Rotella and Simon E Ward, Eds.; Oxford: Elsevier, 2017, vol. 2, pp. 212-232, http://dx.doi.org/10.1016/B978-0-12-409547-2.12319-4 (book chapter)
2. Liu, Z. Chen, H. Y., Wold, E. A., Zhou, J. Chapter 13: Small-molecule inhibitors of protein-protein interactions. In Comprehensive Medicinal Chemistry III (ISBN: 978-0-128-03200-8), Vol 2: Drug Discovery Technologies, 1st ed.; Samuel Chackalamannil, David P Rotella and Simon E Ward, Eds.; Oxford: Elsevier, 2017, vol. 2, pp. 329-353, http://dx.doi.org/10.1016/B978-0-12-409547-2.12326-1 (book chapter)
3. Wold, E. A., Zhou, J. GPCR allosteric modulators: Mechanistic advantages and therapeutic applications. Curr. Top. Med. Chem., 2018, 18 (23), 2002-2006. Free PMC article.
4. Wold, E. A., Chen, J., Cunningham, K. A., Zhou J. Allosteric modulation of class A GPCRs: Targets, agents, and emerging concepts. J. Med. Chem., 2019, 62 (1), 88-127. PMCID: PMC6556150. [Paper in a Special Issue on Allosteric Modulators of Drug Targets]
5. Zhou, J., Cunningham, K. A. Positive allosteric modulation of the 5-HT_{2C} receptor: Implications for neuropsychopharmacology and neurotherapeutics. Neuropsychopharmacology, 2019, 44 (1), 230-231. PMCID: PMC6235849.
6. Zhou, J., Wild, C. GPCR drug discovery: Emerging targets, novel approaches and future trends. Curr. Top. Med. Chem., 2019, 19 (16), 1363-1364. PMCID: PMC6905493.
7. Wold, E. A., Wild, C, Cunningham, K. A., Zhou, J. Targeting the 5-HT_{2C} receptor in biological context and the current state of 5-HT_{2C} receptor ligand development. Curr. Top. Med. Chem., 2019, 19 (16), 1381-1398. PMCID: PMC6761005.
8. Wild, C. T., Miszkiel, J. M., Wold, E. A., Soto, C. A., Ding, C., Hartley, R. M., White, M. A., Anastasio N. C., Cunningham, K. A., Zhou, J. Design, synthesis, and characterization of 4-undecylpiperidine-2-carboxamides as positive allosteric modulators of the serotonin (5-HT) 5-HT_{2C} receptor. J. Med. Chem. 2019, 62 (1), 288-305. PMCID: PMC6533912. [Paper of a Special Issue on Allosteric Modulators of Drug Targets]
9. Wold, E. A., Garcia, E. J., Wild, C. T., Miszkiel, J. M., Soto, C. A., Chen, J., Pazdrak, K., Fox, R. G., Anastasio, N. C., Cunningham, K. A., Zhou, J. Discovery of 4-phenylpiperidine-2-carboxamide analogues as serotonin 5-HT_{2C} receptor positive allosteric modulators with enhanced drug-like properties. J. Med. Chem. 2020, 63 (14), 7529-7544. Epub 2020 Jul. 7. PMCID: In progress.
10. De Deurwaerdere, P., Chagraoui, A., Cunningham, K. A. Editorial: Contemporary perspective on 5-HT2C receptor function and its pharmacological targeting. Front. Pharmacol. 11:606414, 2020. PMCID:PMC7724505.
11. Barnes, N. M., Ahern, G. P., Becamel, C., Bockaert, J., Camilleri, M., Chaumont-Dubel, S., Claeysen, S., Cunningham, K. A, et al. International Union of Basic and Clinical Pharmacology. CX. Classification of receptors for 5-hydroxytryptamine; pharmacology and function. Pharmacol Rev 2021 73:1-213. PMCID: PMC7770494 (available on 2022-01-01).
12. Muller, C. P., Cunningham, K. A. Handbook of the Behavioral Neurobiology of Serotonin. 2^{nd} Edition. Academic Press, United Kingdom, 2020.

### Patents

1. Zhou, J., Ding, C., Cunningham, K. A., Allosteric modulators of 5-hydroxytryptamine 5-HT_{2C} Receptor (5-HT_{2C}R). U.S. Pat. No. 9,533,973 B2, 1/2/2017. International Publication No. WO2013/086266 A3.

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.

1. A compound according to Formula I: 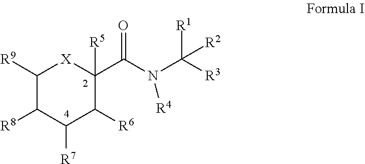
or a pharmaceutically acceptable salt thereof, wherein:
X is —NR— or —O—; wherein R is independently selected from hydrogen, carbamate, or others defined in R¹;
R¹, R², R³ and R⁴ are independently selected from the group consisting of hydrogen, substituted or unsubstituted (C₁-C₁₀) alkyl, substituted or unsubstituted (C₁-C₁₀) alkylene, substituted or unsubstituted (C₁-C₁₀) alkenyl, substituted or unsubstituted (C₁-C₁₀) alkynyl, substituted or unsubstituted (C₁-C₁₀) heteroalkyl, hydroxy (C₁-C₁₀) alkyl, amino (C₁-C₁₀) alkyl, (C₁-C₁₀) alkoxy, (C₁-C₁₀) alkoxy (C₁-C₁₀) alkyl, substituted or unsubstituted cycloalkyl, substituted or unsubstituted heterocyclyl, substituted or unsubstituted aryl, and substituted or unsubstituted heteroaryl; or at least two of any of R¹, R² and R³ taken together form a cycloalkyl or heterocyclyl ring;
R¹, R², R³ and R⁴ are independently substituted with one or more substituents selected from the group consisting of hydrogen, halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl;
R⁵, R⁶, R⁷, R⁸ and R⁹ are independently selected from the group consisting of hydrogen, (C₁-C₁₅) alkyl, (C₁-C₁₅) alkoxy, substituted or unsubstituted (C₁-C₁₅) alkyl aryl, substituted or unsubstituted (C₁-C₁₅) alkyl heteroaryl, substituted or unsubstituted cycloalkyl, substituted or unsubstituted heterocyclyl, substituted or unsubstituted aryl, and substituted or unsubstituted heteroaryl;
with the proviso that when each of R², R³, R⁴, R⁵, R⁶, R⁸ and R⁹ is hydrogen and R⁷ is —CH₂CH₂-phenyl, then R¹ is not morpholino-(CH₂)ₘ—, where m is 2 or 3; and
with the proviso that when each of R³, R⁴, R⁵, R⁶, R⁸ and R⁹ are hydrogen and R⁷ is —CH₂CH₂-phenyl, then R¹ and R² are not both —CH₂OH.

2. The compound according to claim 1, wherein R⁴ is selected from hydrogen and (C₁-C₁₀) alkyl.

3. The compound according to claim 1, wherein R⁴ is hydrogen.

4. The compound according to claim 1, wherein each of R⁵, R⁶, R⁸ and R⁹ is hydrogen.

5. The compound according to claim 1, wherein R⁴ is taken together with any one of R¹, R², and R³ form a nitrogen-containing heterocycle.

6. The compound of claim 5, wherein the nitrogen-containing heterocycle is selected from pyrrolidine, piperidine, and piperazine.

7. The compound according to claim 1, wherein at least two of R¹, R² and R³ taken together form a cycloalkyl or heterocyclyl ring.

8. The compound according to claim 1, wherein at least two of R¹, R² and R³ taken together form a 5-membered cycloalkyl ring, a 6-membered cycloalkyl ring, a 5-membered heterocyclyl ring, or a 6-membered heterocyclyl ring.

9. The compound according to claim 1, wherein at least one of R¹, R², and R³ is hydrogen.

10. The compound according to claim 1, wherein at least two of R¹, R², and R³ are hydrogen.

11. The compound according to claim 1, wherein R², R³, R⁴, R⁵, R⁶, R⁸ and R⁹ are H and R⁷ is —CH₂CH₂-phenyl.

12. The compound of claim 1, wherein R⁵, R⁶, R⁸ and R⁹ are hydrogen and R⁷ is R⁹ are independently selected from the group consisting of (C₁-C₁₅) alkyl, (C₁-C₁₅) alkoxy, substituted or unsubstituted (C₁-C₁₅) alkyl aryl, substituted or unsubstituted (C₁-C₁₅) alkyl heteroaryl, substituted or unsubstituted cycloalkyl, substituted or unsubstituted heterocyclyl, substituted or unsubstituted aryl, and substituted or unsubstituted heteroaryl, and
wherein the R⁷ group at position 4 of the piperidine ring and the amide group at position 2 of the same piperidine ring are in a cis- or a trans-configuration.

13. The compound according to claim 1, wherein X is NH.

14. The compound of claim 1, wherein the compound has a structure selected from: 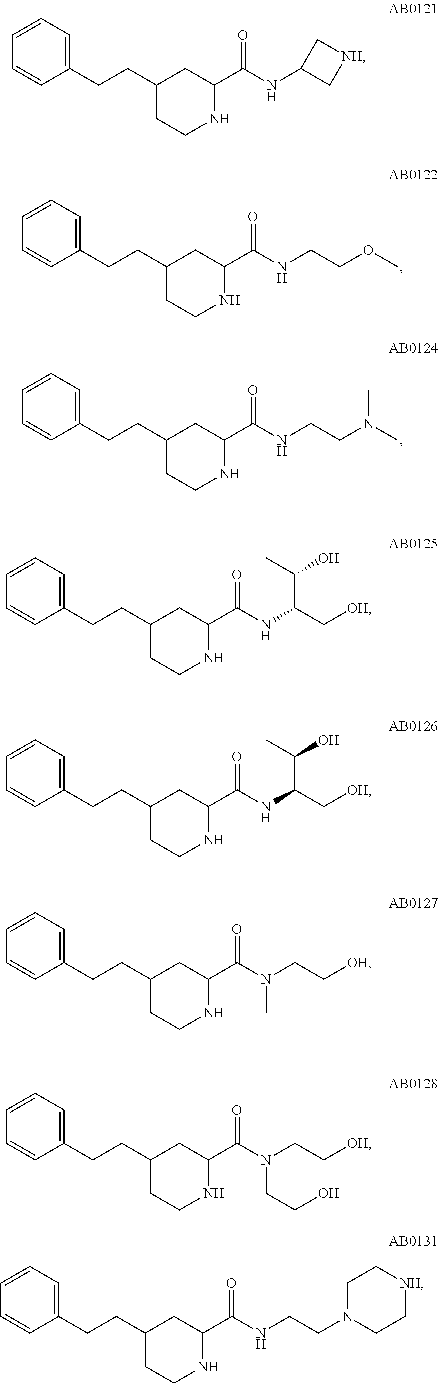 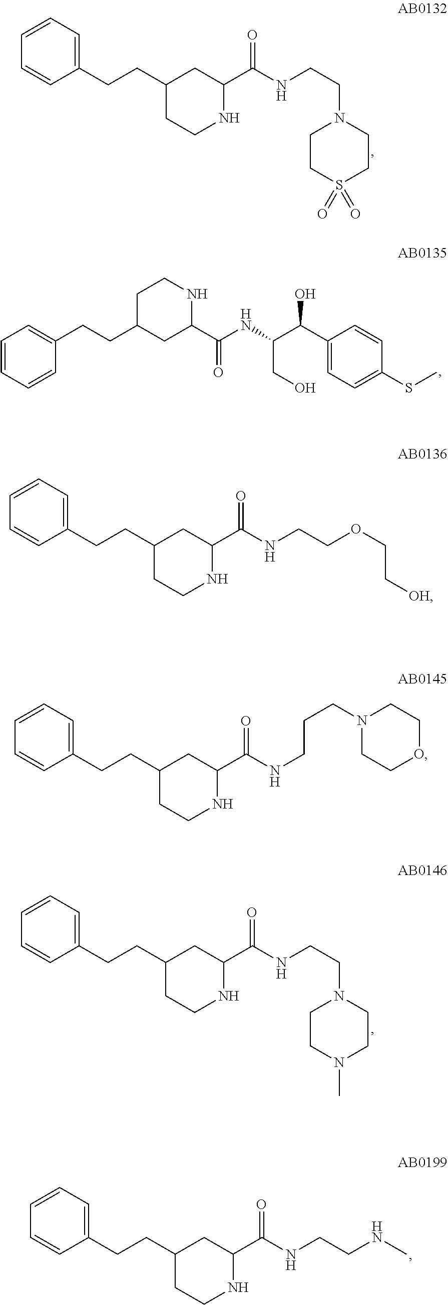 [SMILES: O=C(NCCN1CCS(=O)(=O)CC1)C1CC(CCc2ccccc2)CCN1 ; CSc1ccc([C@H](O)[C@H](CO)NC(=O)C2CC(CCc3ccccc3)CCN2)cc1 ; O=C(NCCOCCO)C1CC(CCc2ccccc2)CCN1 ; O=C(NCCCN1CCOCC1)C1CC(CCc2ccccc2)CCN1 ; CN1CCN(CCNC(=O)C2CC(CCc3ccccc3)CCN2)CC1 ; CNCCNC(=O)C1CC(CCc2ccccc2)CCN1] 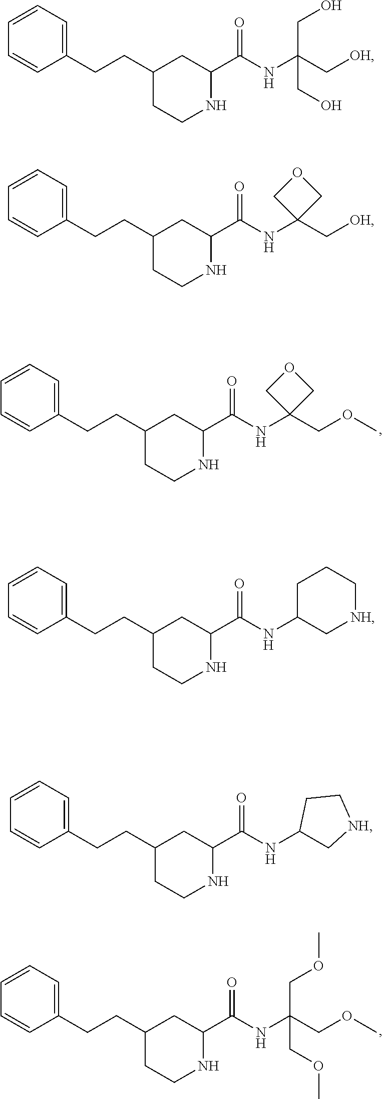 [SMILES: O=C(NC(CO)(CO)CO)C1CC(CCc2ccccc2)CCN1 ; O=C(NC1(CO)COC1)C1CC(CCc2ccccc2)CCN1 ; COCC1(NC(=O)C2CC(CCc3ccccc3)CCN2)COC1 ; O=C(NC1CCCNC1)C1CC(CCc2ccccc2)CCN1 ; O=C(NC1CCNC1)C1CC(CCc2ccccc2)CCN1 ; COCC(COC)(COC)NC(=O)C1CC(CCc2ccccc2)CCN1] 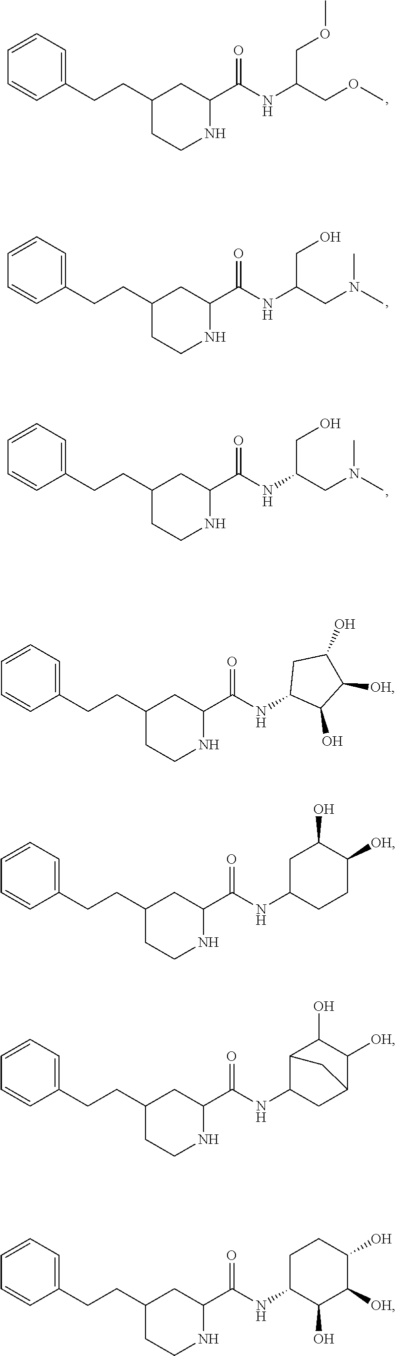 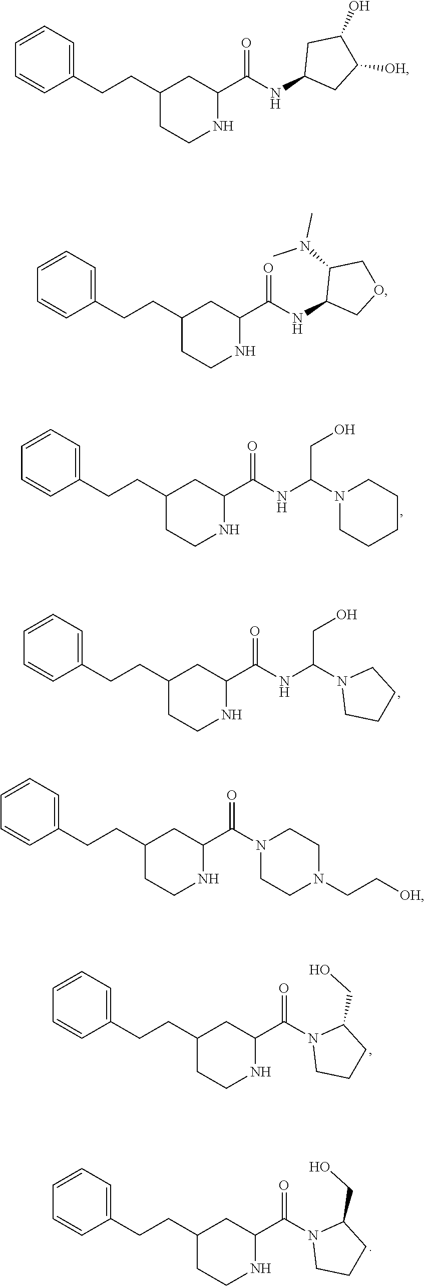
The compound of claim 1, wherein X is O.

15. A method treating a disease that involves modulating hydroxytryptamine 2A receptor and/or a 5-hydroxytryptamine 2C receptor, said method comprising administering an effective amount of the compound according to any of the preceding claims.

16. The method according to claim 15, wherein the compound modulates the 5-hydroxytryptamine 2A receptor.

17. The method according to claim 15, wherein the compound modulates the 5-hydroxytryptamine 2C receptor.

18. The method according to claim 15, wherein the disease is chosen from mood and cognitive disorders (anxiety, depression, schizophrenia), obesity and eating disorders, substance use disorders (SUDs), pain, inflammatory disorders and neurological disorders (such as Alzheimer's, Parkinson's diseases, epilepsy, etc.)

19. The method according to claim 18, wherein the compound modulates the 5-hydroxytryptamine 2A receptor to greater extent than the compound modulates the 5-hydroxytryptamine 2C receptor.

20. The method according to claim 18, wherein the compound modulates the 5-hydroxytryptamine 2C receptor to greater extent than the compound modulates the 5-hydroxytryptamine 2A receptor.
